(19) United States
(12) Patent Application Publication
(10) Pub. No.: US 2025/0115656 A1
(43) Pub. Date: Apr. 10, 2025

(54) Variant Chrmine Proteins Having Accelerated Kinetics and/or Red-Shifted Spectra

(71) Applicants: The Board of Trustees of the Leland Stanford Junior University, Stanford, CA (US); The University of Tokyo, Bunkyo-ku, Tokyo (JP)

(72) Inventors: Karl A. Deisseroth, Stanford, CA (US); Charu Ramakrishnan, San Jose, CA (US); Yoon Seok Kim, Redwood City, CA (US); Yiliu Wang, Redwood City, CA (US); Hideaki Kato, Tokyo (JP); Masahiro Fukuda, Tokyo (JP); Koichiro Kishi, Tokyo (JP)

(21) Appl. No.: 18/730,734

(22) PCT Filed: Jan. 23, 2023
(86) PCT No.: PCT/US2023/061091

§ 371 Date: Jul. 19, 2024

Related U.S. Application Data
(60) Provisional application No. 63/302,419, filed on Jan. 24, 2022.

(51) Int. Cl. C07K 14/705; A01K 67/0275; C12N 15/86; G01N 33/50

(52) U.S. Cl. CPC C07K 14/705; A01K 67/0275; C12N 15/86; G01N 33/5088; A01K 2217/052; A01K 2227/105; A01K 2267/03; C12N 2750/14143

(57) ABSTRACT

Provided is a high-speed variant ChRmine protein having faster kinetic properties compared to a parent ChRmine protein, where the high-speed variant ChRmine protein has one or more amino acid substitutions compared to the parent ChRmine protein. Also provided is a red-shifted variant ChRmine protein having a red-shifted spectrum compared to a parent ChRmine protein, where the red-shifted variant ChRmine protein has one or more amino acid substitutions compared to the parent ChRmine protein. Further provided is a nucleic acid encoding for a variant ChRmine protein disclosed herein as well as a genetically modified cell comprising such nucleic acid. Additionally, provided is an optogenetic method that includes genetically modifying a subject to express in the subject's brain cells a variant ChRmine protein disclosed herein, applying stimulating light to the subject's brain, and imaging the subject's brain.

### CROSS-REFERENCE TO RELATED APPLICATIONS

This application claims priority to U.S. Provisional Application No. 63/302,419 (filed Jan. 24, 2022), which is incorporated herein by reference in its entirety.

### SEQUENCE LISTING

A Sequence Listing is provided herewith as a Sequence Listing XML, “STAN-1931WO_SEQ_LIST” created on Jan. 20, 2023, and having a size of 39,000 bytes. The contents of the Sequence Listing XML are incorporated by reference herein in their entirety.

### INTRODUCTION

ChRmine, which is a pump-like cation-conducting channelrhodopsin, exhibits puzzling properties, such as large photocurrents, red-shifted spectrum, and extreme light-sensitivity. ChRmine and its homologs function as ion channels, but by primary sequence more closely resemble ion pump rhodopsins; mechanisms for passive channel conduction in this family are unknown.

### SUMMARY

This disclosure provides the 2.0-Å resolution cryo-EM structure of ChRmine, revealing architectural features atypical for channelrhodopsins: trimeric assembly, a short transmembrane-helix 3, a twisting extracellular-loop 1, large vestibules within the monomer, and an unprecedented opening at the trimer interface. Based on the structure of ChRmine, three types of proteins were designed that have desirable characteristics in optogenetics: for example, rsChRmine and hsChRmine, having further red-shifted and high-speed properties respectively; and frChRmine, having faster and more red-shifted performance. These proteins can be used in neuroscience research, particularly, using optogenetics.

Accordingly, certain embodiments of the disclosure provide a high-speed variant ChRmine protein having faster kinetic properties compared to a parent ChRmine protein, wherein the high-speed variant ChRmine protein has one or more amino acid substitutions compared to the parent ChRmine protein. Certain embodiments of the disclosure also provide a red-shifted variant ChRmine protein having a red-shifted spectrum compared to a parent ChRmine protein, wherein the red-shifted variant ChRmine protein has one or more amino acid substitutions compared to the parent ChRmine protein. Further embodiments of the disclosure provide a nucleic acid encoding for a variant ChRmine protein disclosed herein as well as a genetically modified cell comprising such nucleic acid. Even further embodiments of the disclosure provide an optogenetic method comprising: genetically modifying a subject to express in the subject's brain cells the variant ChRmine protein disclosed herein, applying stimulating light to the subject's brain, and imaging the subject's brain.

Additional embodiments of the disclosure provide methods comprising: genetically modifying a subject to express in a cell and/or organ the variant ChRmine protein disclosed herein. The methods can further comprise applying stimulating light to the modified cell and/or organ, and imaging the subject's cell and/or organ. The cell and/or organ can belong to the cardiovascular system, the gastrointestinal system, the urinary system, the respiratory system, the reproductive system, the musculoskeletal system, or the pancreatic/endocrine system.

### BRIEF DESCRIPTION OF SEQUENCES

SEQ ID NO: 1: ChRmine protein from Tiarina fusus (GenBank QDS02893.1).
SEQ ID NO: 2: ChRmine protein from Hyphochytrium catenoides (HcKCR1: GenBank MZ826862).
SEQ ID NO: 3: ChRmine protein from Hyphochytrium catenoides (HcKCR2: GenBank MZ826861).
SEQ ID NO: 4: ChRmine protein from Rhodomonas abbreviata (RaCCR1: GenBank QIU80793.1).
SEQ ID NO: 5: ChRmine protein from Rhodomonas salina strain CCMP 1319 (RsCCR1: GenBank QIU80800.1).
SEQ ID NO: 6: ChRmine protein from Rhodomonas abbreviata (RaCCR2: GenBank QIU80796.1).
SEQ ID NO: 7: ChRmine protein from Rhodomonas salina strain CCMP 1319 (RsCCR2: GenBank QIU80801.1).
SEQ ID NO: 8: ChRmine protein from Guillardia theta (GtCCR1: GenBank ANC73520.1).
SEQ ID NO: 9: ChRmine protein from Guillardia theta (GtCCR1: GenBank ANC73518.1).
SEQ ID NO: 10: ChRmine protein from Guillardia theta (GtCCR3: GenBank ANC73519.1).
SEQ ID NO: 11: ChRmine protein from Guillardia theta (GtCCR4: GenBank ARQ20888.1).
SEQ ID NO: 12: ChRmine protein from Halobacterium salinarum NRC-1 (HsBR: PDB 5ZIM).
SEQ ID NO: 13: ChRmine protein from Chlamydomonas reinhardtii (C1C2: PDB: 3UG9).
SEQ ID NO: 14: ChRmine protein from Chlamydomonas reinhardtii (CrChR2: PDB: 6EID).
SEQ ID NO: 15: ChRmine protein from Chlamydomonas reinhardtii, Chlamydomonas noctigama (C1Chrimson: PDB: 5ZIH).
SEQ ID NO: 16: ChRmine protein from Guillardia theta CCMP2712 (GtACR1: PDB: 6CSM).
SEQ ID NO: 17: ChRmine protein from Chlamydomonas reinhardtii (CrChR1: GenBank AAL08946.1).
SEQ ID NO: 18: ChRmine protein from Volvox carteri f. nagariensis (VChR1: GenBank ABZ90900.1).
SEQ ID NO: 19: ChRmine protein from Volvox carteri f. nagariensis (VChR2: GenBank ABZ90902.1).
SEQ ID NO: 20: ChRmine protein from Stigeoclonium helveticum (Chronos: GenBank KF992040.1)
SEQ ID NO: 21: ChRmine protein from Guillardia theta (GtACR2: GenBank AKN63095.1).
SEQ ID NO: 22: ChRmine protein from Rhodomonas lens (RlACR: GenBank APZ76712.1).
SEQ ID NO: 23: A synthetic ChRmine protein (MerMAID1: GenBank QCW06519.1).
SEQ ID NO: 24: ChRmine protein from Pyramimonas melkonianii CCMP772 (PymeACR1: GenBank QNU12853.1).
SEQ ID NO: 25: ChRmine protein from a metagenome (vPyACR_21821: GenBank QNU12854.1).
SEQ ID NO: 26: ChRmine protein from Halobacterium salinarum (HsHR: PDB: 1E12).
SEQ ID NO: 27: ChRmine protein from an uncultured bacterium (BPRMed12: PDB: 4JQ6).
SEQ ID NO: 28: ChRmine protein from Salinibacter ruber (XR: PDB: 3DDL).
SEQ ID NO: 29: ChRmine protein from Dokdonia eikasta KR2 (PDB: 3X3B).
SEQ ID NO: 30: An example of a high-speed variant ChRmine protein.
SEQ ID NO: 31: An example of a red-shifted variant ChRmine protein.
SEQ ID NO: 32: An example of a high-speed and red-shifted variant ChRmine protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Reference to color in the brief description of drawings refers to color drawings, which may be provided based on the jurisdiction.

FIGS. 1A-1G. Cryo-EM structure of ChRmine and comparison with HsBR and C1C2. (A) Cryo-EM density map (top) and ribbon representation (bottom) of ChRmine homotrimer, colored by protomer (red (dark grey shade, largely on the left side of the density map), yellow (lighter grey shade, largely on the right side of the density map), and light green (intermediate grey shade, within the middle portion of the density map)), ATR (green, pointed as shown), and lipid (grey, pointed as shown). (B) Ribbon representation of ChRmine homotrimer, viewed from the intracellular (top, red monomer on the left bottom, yellow monomer on the right bottom, and light green monomer on the top) and extracellular side (bottom, red monomer on the left top, yellow monomer on the right top, and light green monomer on the bottom). (C) Overall structure of ChRmine (left), HsBR (middle), and C1C2 (right), viewed parallel to the membrane (top) and viewed from the intracellular side (bottom). (D) Representative HS-AFM image of ChRmine. (E) ChRmine monomer, viewed parallel to the membrane (left), and from the extracellular side (right). ATR (stick model) is green (pointed as shown), and ECL1 is cyan (pointed as shown). (F-G) ChRmine (red, lighter grey) superimposed onto HsBR (blue, darker grey) (F) and C1C2 (yellow, lighter grey) (G) from two angles.

FIGS. 2A-2D. The Schiff base region. (A) The Schiff base regions of ChRmine (top), HsBR (bottom left), and C1C2 (bottom right). Spheres and black dashed lines represent water molecules and H-bonds, respectively. (B) Photocurrent amplitudes of wild-type (WT) ChRmine and two mutants. Mean±s.e.m. (n=5-6); one-way ANOVA with Dunnett's test. ****p<0.0001. (C) Absorption spectra of ChRmine WT (top), D115N (middle), and D253N (bottom) at pH 7.5 (black) and pH 4.0 (red, grey). (D) Time-series traces of absorption changes for ChRmine WT (top), D115N (middle), and D253N (bottom) at 363-366 (blue, line with the top-most peak), 406 (cyan, line with the second from top peak), 505-520 (green, line with the bottom-most peak), 579-588 nm (red, substantially horizontal line) probe wavelengths.

FIGS. 3A-3I. Ion-conducting pathway within the monomer. (A) The extracellular and intracellular vestibules (EV and IV, respectively) of ChRmine (left), C1C2 (middle), and HsBR (right) within the monomer. Vestibules: solid grey. The intracellular and central constriction sites (ICS and CCS): dashed boxes (green and orange and as pointed, respectively). (B-C) Ion conducting pore and conserved negatively charged residues (E121, E122, E129, E136, and E140) along the ion-conducting pathway of C1C2 (C), and corresponding residues of ChRmine (B). Pores are colored by electrostatic potential. (D) Ion conducting pore and pore-aligning negatively charged residues of ChRmine, C1C2, CrChR2, C1Chrimson, and positively charged residues of GtACR1. Grey mesh: ion-conducting pore. (E) ICS (top) and CCS (bottom) of ChRmine (left) and C1C2 (right). (F) Mutations in constriction sites. Data mean±s.e.m. (n (number of cells)=5-9); one-way ANOVA with Dunnett's test. *p<0.05, ***p<0.001, ****p<0.0001. (G) The superposed intracellular regions of ChRmine (red, lighter grey) and HsBR (blue, darker grey) (left) and intracellular region of ChRmine (right). IV1 and IV2 of ChRmine: grey mesh. (H) The overall structure of the intracellular region of HsBR. The intracellular vestibules of HsBR: grey mesh. (I) Magnified views of the blue boxed region in HsBR as shown in (A). Comparison of the overall structure (left) and key residues (right) of ECL1 between ChRmine (red and as pointed), C1C2 (yellow and as pointed), and HsBR (blue and as pointed). The extracellular cavity of ChRmine: grey mesh. ECL1 of ChRmine adopts the different conformation shown (left), while in HsBR Y79 and R82 efficiently occlude the cavity.

FIGS. 4A-4K. The hydrophilic pore within the trimer interface. (A) Location of the pore within the trimer interface in ChRmine (left) and HsBR (right). The trimer pore pathway is depicted as grey mesh and only two protomers are shown for clarity. The pore in HsBR is hydrophobic and filled with several lipid molecules, but for ChRmine it is hydrophilic and negatively charged. (B) Electrostatic potential surface and cross-section of the ChRmine (left) and HsBR (right). (C) Trimer pore radii of the ChRmine as a function of the distance along the pore axis, calculated with HOLE. (D) Magnified views of the blue-boxed region from (A), the constriction formed by ECL1, from two angles. (E-F) Comparison of ECL of ChRmine with the ion selectivity filter of NavMs (E) and ASIC (F). (G-H) Magnified views of the boxed regions in (A), the hydrogen bond interactions between protomers at the intracellular (G) and extracellular (H) side. (I) Fluorescent size-exclusion chromatography (FSEC) traces of ChRmine WT, S138W, Y156F, and S138W/Y156F mutants, suggesting the destabilization of the trimer by mutations. (J) Photocurrent amplitudes for the mutants shown in (G-H) as well as R136H (as a negative control) (Data are mean±s.e.m. n=4-7; one-way ANOVA followed by Dunnett's test, **p<0.01. (K) Reversal potentials of ChRmine WT, R136H, and S138W mutants, showing that S138W mutation modulates cation selectivity. Data are mean±s.e.m. (n=3-9); unpaired t-test, **p<0.01 and ****p<0.0001. N.S., not significant.

FIGS. 5A-5F. Computational analysis of pore dynamics. (A) Visualization of the three F104 residues that define the trimer pore constriction site from representative frames of the dark and light state channels in simulation. (B) Trimer pore radius was calculated for each of 10 independent 2 μs simulations. Pore radius is larger in light-state simulations than dark-state stimulations (p<0.001, Welch's-test). (C-D) Water permeating the trimer interface during the MD simulation. Representative snapshot with water molecules around the constriction (C) and successive snapshots focusing on one water molecule (D). (E) The ChRmine monomer pore opens wider in light-state simulations (13-cis-retinal and protonated D115) compared to dark-state simulations (all-trans-retinal and deprotonated D115) (p<0.001, Welch's-test). Average minimum monomer pore radius for the three monomers was calculated for each of 10 independent 2 μs simulations. (F) The pore radius is larger in the light-state simulations than the dark-state simulations as shown through representative frames from the two simulation conditions. Rotation of D115 and isomerization of the retinal opens the internal monomer space.

FIGS. 6A-6K. Structure-guided design of ChRmine variants. (A) Residues comprising the EV (dark grey surface) of ChRmine. Dashes denote H-bonds. (B) RBPs of ChRmine (top left), C1C2 (top right) and HsBR (bottom). Key amino acids and all-trans retinal molecules depicted by stick model. (C) On- (left) and off- (right) kinetics (n=4-23). (D) Summary of photocurrent ratios (n=4-16). (E) Voltage clamp traces of WT- (top) and rsChRmine- (bottom) expressing neurons stimulated by indicated light wavelengths. (F) Example traces of opsins. (G) Normalized action spectra of WT and rsChRmine (n=16 for WT and 15 for rs). (H) 2P action spectra of WT (n=6) and rs (n=5). (I) Schematic of brain slice physiology. (J) Spike probability versus pulse width in slices (5 Hz/4 s, 1 mW/mm², n=5 for WT, n=6 for rs). (K) Spike probability versus light power in slices (at 5 Hz for 5 s, pulse width=5 ms, n=5 for WT, n=6 for rs). All data mean±s.e.m.; sample size n denotes number of cells. Wilcoxon ranksum test for G and Kruskal-Wallis test with Dunn's test for the rest. *p<0.05, **p<0.01, ***p<0.001, ****p<0.0001.

FIGS. 7A-7R. In vitro demonstration of rsChRmine application. (A) Schematic: all optical physiology. (B) Representative traces of XCaMP-G response to orange (585 nm) or red (635 nm) light stimulation in WT (left) or rsChRmine (right) neurons. (C) Peak XCaMP-G responses to orange and red light (n=14 for rs; n=15 for WT, two-tailed Mann-Whitney U test). (D) Peak XCaMP-B responses to blue (435 nm; left), cyan (488 nm; middle), and green (570 nm; right) light stimulation (n=31 for rs, n=32 for WT, two-tailed Mann-Whitney U test). (E) Summary of rise and decay kinetics of XCaMP-G transients (n=14 for hs; n=17 for rs; n=24 for WT, one-way ANOVA with Turkey's test). (F) Peak XCaMP-G responses to 585 nm light stimulation (2.0 mW/mm²) (n=14 for hs and 10 for WT). All data mean±s.e.m.; sample size n denotes number of cells. *p<0.05, **p<0.01, ***p<0.001, ****p<0.0001. (G) Schematic: FIP rig for simultaneous Ca²⁺ recording and optogenetic stimulation. (H) Schematic: virus delivery and fiber placement in mPFC. (I) Expression of GCaMP6m and rsChRmine in neurons. (J) GCaMP6m traces in response to 594 nm stimulation in freely moving mice expressing opsins. (n=4 mice, 4 trials per mouse). (K) Left: mean response to various light powers. Right: EPD50. (L) Quantification of baseline GCaMP6m fluorescence in rsChRmine, WT ChRmine, and ChrimsonR expressing mice. (M) Mean amplitudes evoked at 470 nm normalized to the peak amplitude evoked at 594 nm stimulation for mice expressing each opsin. (N) GCaMP6m fluorescence at the start of imaging at different 470 nm light powers. (O) GCaMP6m traces in response to 720 nm (top) and 750 nm (bottom) stimulation in freely moving mice expressing opsins. (n=4 mice, 4 trials per mouse). (P) Mean response to 594, 720, and 750 nm stimulation of the indicated opsins. (Q and R), Pyr to PV (Q) and PV to Pyr (R) Ca²⁺ recordings. Left, schematic of transgene expression in mPFC PV and CaMKIIα-positive Pyr neurons. Right, representative averaged simultaneous 2-color photometry traces aligned to the start of 594 nm stimulation (green: GCaMP6, blue: XCaMP-B). All data mean (curves)±s.e.m. (shading around curves); sample size n denotes number of cells unless otherwise noted. One-way ANOVA with Turkey's test. *p<0.05, **p<0.01, ***p<0.001, ****p<0.0001.

FIGS. 8A-8F. Structure-based sequence alignment, phylogenetic tree, and predicted structure of ChRmine. (A) The sequences are ChRmine (GenBank QDS02893.1; SEQ ID NO: 1), HcKCR1 (GenBank MZ826862; SEQ ID NO: 2) (Govorunova et al., 2021), HcKCR2 (GenBank MZ826861; SEQ ID NO: 3) (Govorunova et al., 2021), RsCCR1 (GenBank QIU80800.1; SEQ ID NO: 5), RaCCR1 (GenBank QIU80793.1; SEQ ID NO: 4), RsCCR2 (GenBank QIU80801.1; SEQ ID NO: 7), RaCCR2 (GenBank QIU80796.1; SEQ ID NO: 6), GtCCR1 (GenBank ANC73520.1; SEQ ID NO: 8), GtCCR2 (GenBank ANC73518.1; SEQ ID NO: 9), GtCCR3 (GenBank ANC73519.1; SEQ ID NO: 10), GtCCR4 (GenBank ARQ20888.1; SEQ ID NO: 11), HsBR (PDB: 5ZIM; SEQ ID NO: 12) (Hasegawa et al., 2018), C1C2 (PDB: 3UG9; SEQ ID NO: 13) (Kato et al., 2012), CrChR2 (PDB: 6EID; SEQ ID NO: 14) (Volkov et al., 2017), C1Chrimson (PDB: 5ZIH; SEQ ID NO: 15) (Oda et al., 2018), GtACR1 (PDB: 6CSM; SEQ ID NO: 16) (Kim et al., 2018), respectively. The sequence alignment was created using PLOMALS3D (Pei et al., 2008) and ESPript 3 (Robert and Gouet, 2014) servers. Secondary structure elements for ChRmine are shown as coils. Positive and negative charged residues are highlighted (in blue and red, respectively). Stars represent the DTD motif. The ECL1 of ChRmine is highlighted (colored light blue). The counterions are also highlighted (colored orange). (B) An unrooted phylogenetic tree was drawn for representative microbial rhodopsins using the Neighbor-Joining method (Saitou and Nei, 1987), and 1,000 bootstrap replicates. Evolutionary analyses were conducted in MEGA7 (Kumar et al., 2016). White circles represent bootstrap values>85%. (C-F) Five predicted models of ChRmine, generated using locally-installed AlphaFold2. The ribbon representations are highlighted (colored by the pLDDT score (low: red, high: cyan)). (C) Plots of pLDDT score. (D) The best predicted model superimposed onto the cryo-EM structure (yellow). (E-F) The detailed comparison of ECL1 (E) and the Schiff base region (F) between the five predicted models and cryo-EM structure. Notably, the C-terminal region of ECL1, including D115, has high pLDDT scores, but the conformation of D115 is not correctly predicted.

FIGS. 9A-9R. Cryo-EM analysis of ChRmine and ChRmine-Fab02 complex. (A-C) Panels corresponding to ChRmine alone. Representative SEC trace with SDS-PAGE as inset (A), representative cryo-EM micrograph (B), and 2D-class averages (C). (D-F) Panels corresponding to the ChRmine-Fab02 complex. Representative SEC trace with SDS-PAGE as inset (D), representative cryo-EM micrograph (E), and 2D-class averages (F). (G) Low-resolution reconstruction of ChRmine alone. (H) Data processing workflow of ChRmine-Fab02 complex. Final cryo-EM map colored by local resolution. (I) Fourier Shell Correlation (FSC) between the two independently refined half-maps. (J) FSC between the model and the map calculated for the model refined against the full reconstruction. (K-N) Cryo-EM density (FSC-weighted sharpened map calculated by RELION3.1.1) and model for ChRmine, lipids (K), the retinal binding pocket (L), the Schiff base region (M), twisted ECL1 (N). (O-P) Density and model near ECL1 region. FSC-weighted sharpened map calculated by RELION3.1.1 (blue) and Fₒ−Fₑ map calculated by Servalcat (green). Positive Fₒ−Fₑ difference densities (4.3σ, where σ is the standard deviation within the mask) are observed near nitrogen atoms, suggesting that these densities represent hydrogen atoms. (Q-R) Possible signal of early photo-intermediate. (Q) Density and model in the retinal binding pocket region. Maps (Blue and green) are FSC-weighted sharpened map calculated by RELION3.1.1 and Fₒ−Fₑ maps calculated by Servalcat, respectively. Positive and negative Fₒ—Fₑ difference density pairing (±3.7σ, where σ is the standard deviation within the mask) is observed around W223, suggesting that this density contains information regarding a small population of the early intermediate state, and that W223 moves upward early in the photocycle. (R) Magnified views of (Q).

FIGS. 10A-10L. Spectroscopic, structural, and HPLC characterization of the ChRmine and ChRmine-Fab02 complex, related to FIG. 1. (A) FSEC screening of Fab fragments. (B) Titration of Fab02 fragment against ChRmine. (C and D) Transient absorption spectra of ChRmine WT (C) and the ChRmine-Fab02 complex (D) excited at λ_{exc}=532 nm. (E) Time series traces of absorption changes of ChRmine WT (solid line) and the ChRmine-Fab02 complex (right) at 363 (blue), 406 (cyan), 520 (green), 588 nm (red) probe wavelengths. (F) Photocycle scheme of ChRmine determined by flash photolysis shown in (E). (G) The absorption spectra of the initial state (grey, ChRmine), L₁ (orange, K/L₁), M₁/L₂ (green), and M₂ (blue) of ChRmine calculated from the decay-associated-spectra of transient absorption changes (Inoue et al., 2013). (H) Cryo-EM map of ChRmine-Fab02 complex. (I) Interactions between ChRmine and Fab02. (J-K) HPLC analysis of the chromophore configuration of ChRmine. (J) Representative HPLC profiles of the chromophore of ChRmine in the dark (top), under illumination (middle), and after light adaptation (bottom). “at”, “11”, and “13” indicate the peak of all-trans-, 11-cis-, and 13-cis-retinal oximes, respectively. (K) Calculated composition of all-trans- and 13-cis-retinal oximes. Data are presented as mean±s.e.m. (n=3). Purified samples of ChRmine were prepared with additional supplementation of all-trans-retinal. Green light (530±5 nm) was used for illumination. Light adaptation was achieved by illumination for 1 min followed by incubation in the dark for 2 min. (L) Lifetime of each intermediate in ChRmine WT and mutants.

FIGS. 11A-11F. Comparison of the Schiff base regions, related to FIG. 2. (A) Concept of the Schiff base counterions and proton acceptor. (B) The superposed Schiff base region of ChRmine (red) and HsBR (blue). Spheres (red and blue) represent water molecules of ChRmine and HsBR, respectively. Black dashed lines represent hydrogen bonds. (C) Superposed Schiff base region of ChRmine (red) and representative microbial rhodopsins (CrChR2 (Volkov et al., 2017), C1Chrimson (Oda et al., 2018), GtACR1 (Kim et al., 2018), schizorhodopsin 4 (SzR4) (Higuchi et al., 2021), KR2 (Kato et al., 2015b), HsHR (Kolbe et al., 2000), NpSRII (Gordeliy et al., 2002), heliorhodopsin (Shihoya et al., 2019)), displayed with high transparency except for ChRmine. (D) List of the Schiff base regions of representative microbial rhodopsins. (E-F) pH titration experiment of WT ChRmine. (E) Absorption spectra of WT ChRmine measured from pH 2.2 to 10.0. (F) The λₘₐₓ value at each pH.

FIGS. 12A-12D. Electrophysiology, related to FIGS. 2-4. (A) Representative traces of ChRmine WT and 13 mutants expressed in HEK293 cells by lipofectamine transfection, measured at −70 mV holding potential in voltage-clamp. Traces were recorded while cells were stimulated with 1.0 s of 1 mW mm⁻² irradiance at 580 nm. (B-D) Summary of the steady-to-peak ratio of photocurrents (B), τ_{off} of channel closing (C), and τ_{off} of channel desensitization (=τ_{desen}) (D). Mutants are categorized as the mutants of counterions, central constriction site (CCS), intracellular constriction site (ICS), and the trimer interface. Data are mean±s.e.m (n=3-9); one-way ANOVA followed by Dunnett's test. **p<0.01, ***p<0.001, and ****p<0.0001.

FIGS. 13A-13H. Detailed characterization of rsChRmine, related to FIG. 6. (A) The current-voltage dependence of non-normalized (left) and normalized (right) peak photocurrents of ChRmine at different voltages from −75 mV to 45 mV stimulated with 1 s of 1 mW mm⁻² irradiance at 580 nm. (n=6-10). (B and C) Action spectra of single (B) and double (C) mutants of residues in the RBP. (n=4-16). (D) Peak photocurrents for RBP mutants. (n=4-16, Kruskal-Wallis test with Dunn's test, Asterisks denote comparisons to WT ChRmine, *p<0.05, **p<0.01, ****p<0.0001). (E) Normalized photocurrent versus light power in cultured neurons with cyan (470 nm), orange (585 nm) and red (650 nm) light (n=5 for WT and 7 for rs). (F) Effective power density (EPD50) of WT and rsChRmine (two-tailed student's t-test, n=5 for WT and 7 for rs, *p<0.05, **p<0.01, ***p<0.001, ****p<0.0001). (G and H) Spike probability versus light intensity (G) and pulse width (H) in cultured neurons expressing opsins (n=6 for WT and 7 for RS, Kruskal-Wallis test with Dunn's test). All data are mean±s.e.m., and sample size n denotes the number of cells unless otherwise noted. *p<0.05, **p<0.01, ***p<0.001, ****p<0.0001.

FIGS. 14A-14H. Detailed comparison of ChRmine with ChroME variants, related to FIG. 6. (A) Confocal images of WT ChRmine, hsChRmine, rsChRmine, ChroME2f, and ChroME2s expressing cultured neurons. Scale bar=10 μm. (B) Summary of on (left) and off (right) kinetics of ChRmine and ChroME variants (n=3-23, Kruskal-Wallis test with Dunn's test. Asterisks denote comparisons to WT ChRmine, *p<0.05, **p<0.01, ***p<0.001, ****p<0.0001). (C) Example current clamp traces of hsChRmine, WT ChRmine and ChroME2f expressing neurons with light stimulation. (D) Summary of opsin spike fidelity (n=5 for all mutants, Kruskal-Wallis test with Dunn's test. Asterisks denote comparisons between ChroME2s and WT ChRmine, *p<0.05, **p<0.01, ***p<0.001). (E and F) Normalized (E) and peak photocurrent (F) action spectra of ChRmine and ChroME variants (n=4-16). (G and H) Example traces of rsChRmine (G) and normalized action spectra (H) of ChRmine and ChroME variants under two-photon excitation (n=4-6). All data are mean±s.e.m., and the sample size n denotes the number of cells unless otherwise noted. For WT ChRmine (compared here under identical conditions alongside other ChRs in the same preparation for rigorous and unbiased comparison of performance regarding photocurrents, action spectra, spike frequency-response, and EPD50), we observed slightly lower spike fidelity than had been observed in (Marshel et al., 2019); this can be seen with preparation-to-preparation variance of opsin expression level in cultured neurons.

### DETAILED DESCRIPTION

Provided are pump-like cation-conducting channelrhodopsins. Three types of proteins were designed that have desirable characteristics in optogenetics. Examples of these proteins include: rsChRmine and hsChRmine, having further red-shifted and high-speed properties respectively; and frChRmine, having faster/accelerated kinetics and greater red-shifted performance compared to rsChRmine. These proteins can be used in neuroscience research, particularly, using optogenetics.

Before the present invention is described in greater detail, it is to be understood that this invention is not limited to embodiments described, as such may vary. It is also to be understood that the terminology used herein is for the purpose of describing embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As used herein and in the appended claims, the singular forms “a,” “an,” and “the” include plural referents unless the context clearly dictates otherwise. For example, reference to “a protein” includes a plurality of such proteins and reference to “a mutation” includes reference to one or more discrete mutations, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The terms “kinetics” or “kinetic property/properties” as used herein in reference to a ChRmine protein refer to the rates of opening and closing of channelrhodopsin ion channels of the ChRmine protein. A ChRmine protein having higher rates of opening and closing of channelrhodopsin ion channels compared to another ChRmine protein is said to have faster or accelerated kinetics or kinetic property/properties compared to the other ChRmine protein.

“A high-speed variant ChRmine protein” has accelerated kinetic property/properties, i.e., faster kinetic property/properties, compared to a parent ChRmine protein used to produce the high-speed variant ChRmine protein. A high-speed variant ChRmine protein can have faster kinetic properties compared to a parent ChRmine protein used to produce the high-speed variant ChRmine protein by 10% or more, 20% or more, 30% or more, 40% or more, 50% or more, 60% or more, 70% or more, 80% or more, 80% or more.

ChRmine kinetics can be expressed as “rise time,” tau off (τ_{off}), or a combination of both.

“Rise time” (tpeak) is the time-to-peak from the cessation of the light stimulus to the time point at which maximal-amplitude fluorescence was reached. For example, a wild-type ChRmine protein can have the time-to-peak of between 15-20 ms, whereas the corresponding high-speed variant ChRmine protein can have the time-to-peak of 5-10 ms. Thus, compared to a parent ChRmine protein used to produce a high-speed variant ChRmine protein, the high-speed variant ChRmine protein can have time-to-peak reduced by 10% or more, 20% or more, 30% or more, 40% or more, 50% or more, 60% or more, 70% or more, 80% or more, or 90% or more.

Also, as an example, a wild-type ChRmine protein can have τ_{off} between 50-150 ms, whereas the corresponding high-speed variant ChRmine protein can have τ_{off} between 20-50 ms. Thus, compared to a parent ChRmine protein used to produce a high-speed variant ChRmine protein, the high-speed variant ChRmine protein can have τ_{off} reduced by 10% or more, 20% or more, 30% or more, 40% or more, 50% or more, 60% or more, 70% or more, 80% or more, or 90% or more.

“Red-shifted spectrum” as used herein in reference to a ChRmine protein refers to red-shifted absorption by a ChRmine protein, for example, a variant ChRmine protein, compared to another ChRmine protein, for example, a parent ChRmine protein. A ChRmine protein having red-shifted absorption compared to a parent ChRmine protein is referenced herein as “red-shifted variant.”

The wavelength eliciting maximum photocurrent is the same for both opsin proteins but the maximum photocurrent is different for all other wavelengths in a parent ChRmine protein as compared to the corresponding red-shifted variant ChRmine protein. For example, photocurrents can be lower for a red-shifted variant ChRmine protein to the corresponding parent ChRmine protein at 380, 440, 480 nm, this indicating photocurrent reduction at blue wavelengths, which represents red shifting, and photocurrents are higher at 650 nm, which also represents red shifting.

For example, a parent ChRmine protein can have the following maximum photocurrents: 380 nm: 0.49, 440 nm: 0.78, 480 nm: 0.94, 513 nm: 1, 580 nm: 0.82, 650 nm: 0.18. These absorption values are normalized to the maximum photocurrent, which is at 513 nm. A corresponding red-shifted variant ChRmine protein can have the following maximum photocurrents: 380 nm: 0.41, 440 nm: 0.50, 480 nm: 0.80, 513 nm: 1, 580 nm: 0.81, and 650 nm: 0.31. Again, these absorption values are normalized to the maximum photocurrent, which is at 513 nm.

Thus, at wavelengths lower than the wavelength that provides maximum photocurrent, compared to the maximum photocurrents of a parent ChRmine protein, a red-shifted variant ChRmine protein can have the maximum photocurrent reduced by 10% or more, 20% or more, 30% or more, 40% or more. On the other hand, at wavelengths higher than the wavelength that provides maximum photocurrent, compared to the maximum photocurrents of a parent ChRmine protein, a red-shifted variant ChRmine protein can have the maximum photocurrent increased by 10% or more, 20% or more, 30% or more, 40% or more.

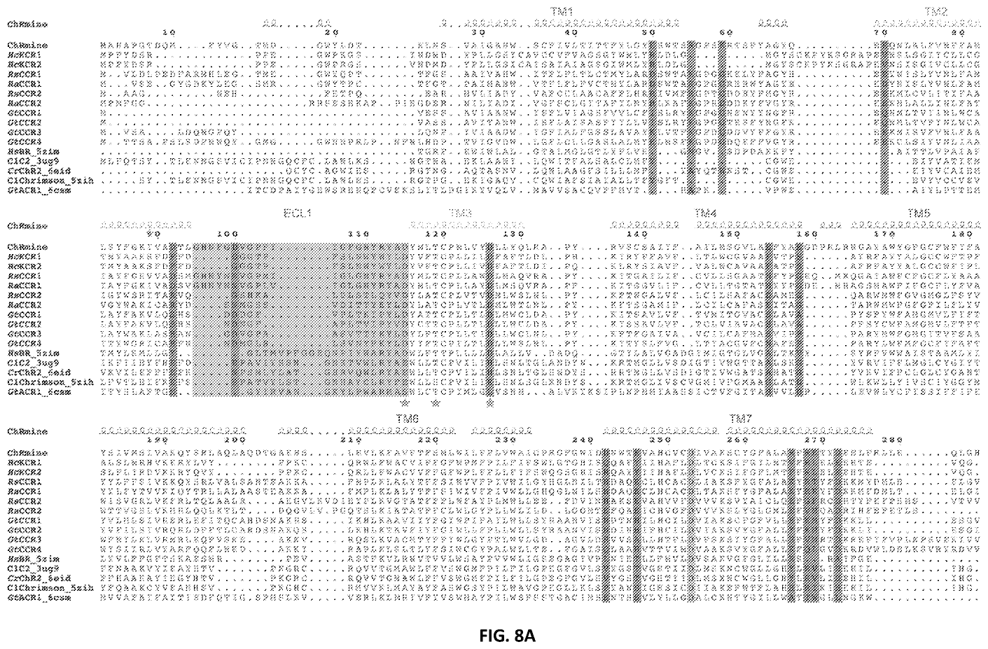

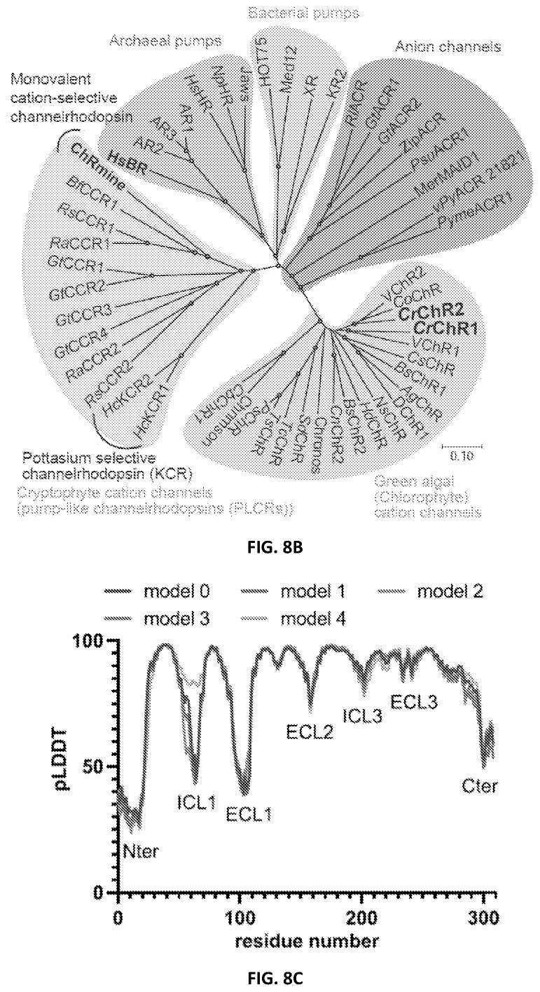

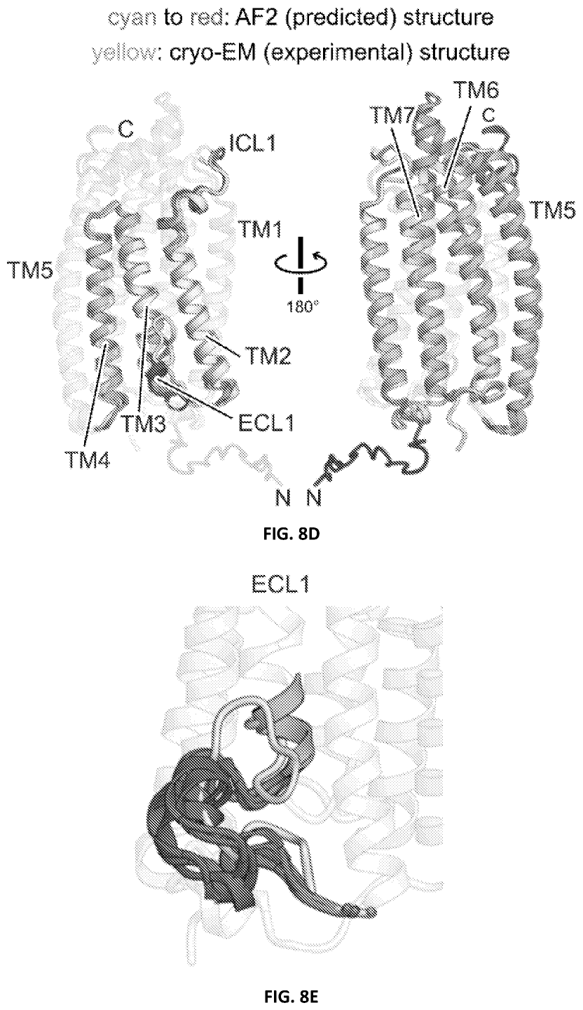

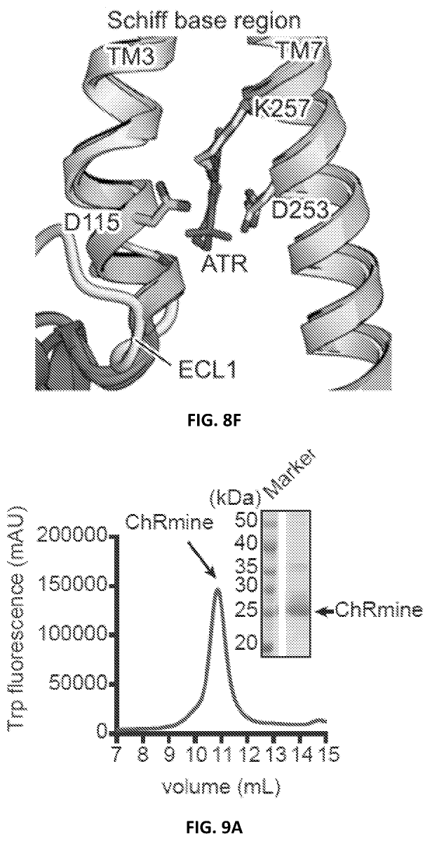

A “parent ChRmine protein” as used herein refers to a wild-type or naturally occurring ChRmine protein. In some instances, a parent ChRmine protein can be mutated to produce a variant ChRmine protein. A parent protein can be a wild-type or naturally occurring ChRmine protein or a homolog thereof. Non-limiting examples of such parent ChRmine proteins are provided in FIG. 8A and SEQ ID NOs: 1 to 29. Additional examples of ChRmine proteins that could be used as parent ChRmine proteins are well known in the art and use of such ChRmine proteins to produce variant ChRmine proteins as disclosed herein is within the purview of the disclosure. Certain such examples include CrChR1 (GenBank AAL08946.1; SEQ ID NO: 17), VChR1 (GenBank ABZ90900.1; SEQ ID NO: 18), VChR2 (GenBank ABZ90902.1; SEQ ID NO: 19), Chronos (GenBank KF992040.1; SEQ ID NO: 20), GtACR2 (GenBank AKN63095.1; SEQ ID NO: 21), R1ACR (GenBank APZ76712.1; SEQ ID NO: 22), MerMAID1 (GenBank QCW06519.1; SEQ ID NO: 23) (Oppermann et al., 2019), PymeACR1 (GenBank QNU12853.1; SEQ ID NO: 24) (Rozenberg et al., 2020), vPyACR_21821 (GenBank QNU12854.1; SEQ ID NO: 25) (Rozenberg et al., 2020), HsHR (PDB: 1E12; SEQ ID NO: 26) (Kolbe et al., 2000), BPRMed12 (PDB: 4JQ6; SEQ ID NO: 27) (Ran et al., 2013), XR (PDB: 3DDL; SEQ ID NO: 28) (Luecke et al., 2008), KR2 (PDB: 3X3B; SEQ ID NO: 29) (Kato et al., 2015b).

“A homologous protein or a protein homolog” of a protein is another protein having similar or identical function and a similar primary, secondary, and/or tertiary structures. Typically, homologous proteins or protein homologs have substantial sequence similarity, for example, at least 80%, at least 85%, at least 90%, at least 95%, or at least 99% sequence identity. Certain non-limiting examples of ChRmine protein homologs are provided as SED ID NOs: 1 to 29. Sequence alignment of some of these proteins is provided in FIG. 8A. Additional examples of ChRmine protein homologs are well known in the art and use of such ChRmine proteins to produce variant ChRmine proteins as disclosed herein is within the purview of the disclosure. For example, a person of ordinary skill in the art can select a wild-type, naturally occurring, or artificial/mutated ChRmine protein and make amino-acid substitutions, particularly conservative amino acid substitutions, which do not affect the function or structure of the protein thereby producing another ChRmine protein homolog. Certain examples of conservative amino acid substitutions, i.e., substitution within the same class of amino acids, are provided in Table 1 below. Homologs produced after such conservative amino acid substitutions can then be further modified according to this disclosure to produce a high-speed variant ChRmine protein or a red-shifted variant ChRmine protein.

TABLE 1

| | Amino acids that constitute | |
|---|---|---|
| | conservative amino acid | |
| Class | substitutions | 1-letter code |
| Aliphatic | Glycine, Alanine, Valine, | G, A, V, L, I |
| | Leucine, Isoleucine | |
| Hydroxyl or | Serine, Cysteine, Threonine, | S, C, U, T, M |
| sulfur/selenium- | Methionine | |
| containing | | |
| Cyclic | Proline | P |
| Aromatic | Phenylalanine, Tyrosine, Tryptophan | F, Y, W |
| Basic | Histidine, Lysine, Arginine | H, K, R |
| Acidic and their | Aspartate, Glutamate, Asparagine, | D, E, N, Q |
| amides | Glutamine | |

The phrase “a corresponding residue in a homolog of ChRmine protein” refers to a residue in a homolog of ChRmine protein that aligns with a reference residue in a ChRmine protein, for example, as shown in FIG. 8A. For example, histidine in the 33^{rd} position of ChRmine protein shown in FIG. 8A corresponds to a serine residue in HcKCR1 protein as shown in this Figure. Similarly, the 33^{d} position of ChRmine protein shown in FIG. 8A corresponds to aspartate, asparagine, alanine, leucine, or glutamine in certain other ChRmine homologs. A person of ordinary skill in the art can readily identify a residue in a homolog of ChRmine protein that corresponds to a reference residue in ChRmine protein by producing and analyzing a sequence alignment of the amino acid sequences, such as the one provided in FIG. 8A.

### Variant ChRmine Proteins

Provided are variants of ChRmine protein, which is a pump-like cation-conducting channelrhodopsin.

In certain embodiments, a variant ChRmine protein is a high-speed variant ChRmine protein having faster kinetic properties compared to a parent ChRmine protein, wherein the high-speed variant ChRmine protein has one or more amino acid substitutions compared to the parent ChRmine protein.

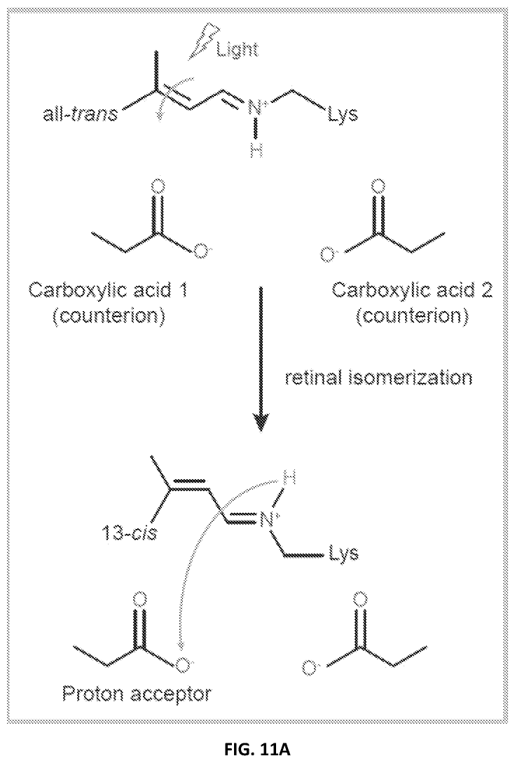

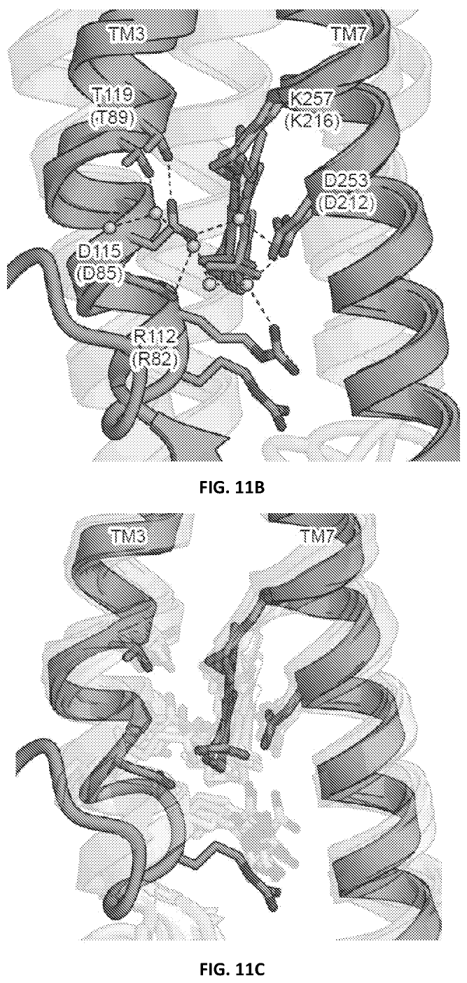

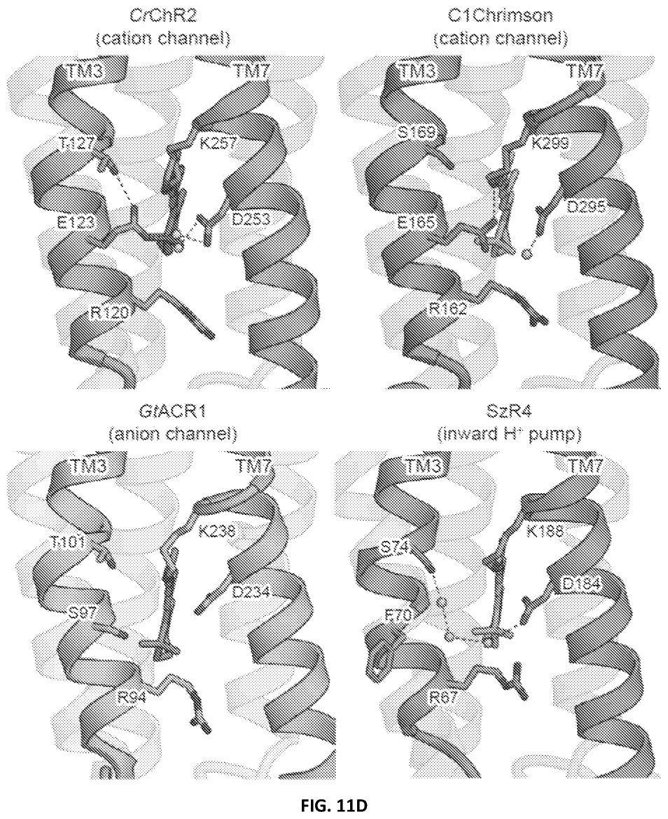

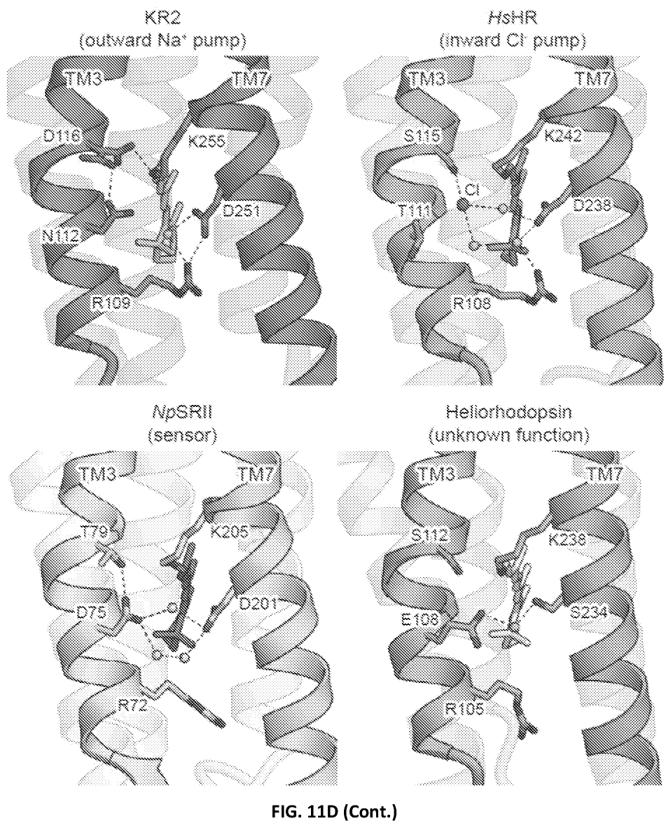

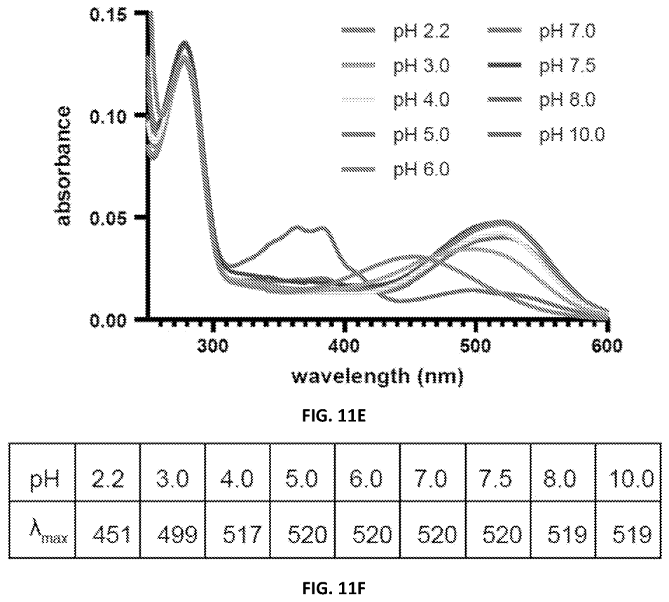

In some cases, a high-speed variant ChRmine protein can have one or more amino acid substitutions in Schiff base counterion. Certain such amino acids are identified in FIGS. 8A and 11A.

A high-speed variant ChRmine protein can also have one or more amino acid substitutions that alter the pore electrostatic potential of a parent protein. Certain such amino acid substitutions include substitutions in one or more of: 33^{rd} histidine or a corresponding position; 92^{nd} aspartate or a corresponding position; 154^{th} glutamate or a corresponding position; 158^{th} glutamate or a corresponding position, 242^{nd} aspartate or a corresponding position, and 246^{th} glutamate or a corresponding position. Each of these positions can be substituted with any other amino acid. Substitutions at the 33^{rd} histidine or a corresponding position can be with a histidine (when the corresponding amino acid is not histidine), arginine, or lysine. Substitution in the 92^{nd} aspartate or a corresponding position; 154^{th} glutamate or a corresponding position; 158^{th} glutamate or a corresponding position, 242^{nd} aspartate or a corresponding position, and 246^{th} glutamate or a corresponding position can be with aspartate, glutamate, asparagine, or glutamine.

For example, substitution at the 33^{rd} histidine or a corresponding position can be with arginine. Substitution at the 33^{rd} histidine or a corresponding position can also be with lysine. When the amino acid corresponding to the 33^{rd} histidine is not histidine, such amino acid can be substituted with histidine.

Substitution in the 92^{nd} aspartate or a corresponding position can be with aspartate. Substitution in the 92^{nd} aspartate or a corresponding position can also be with glutamate. Substitution in the 92^{nd} aspartate or a corresponding position can be with asparagine. Substitution in the 92^{nd} aspartate or a corresponding position can also be with glutamine.

Substitution in the 154^{th} glutamate or a corresponding position can be with aspartate. Substitution in the 154^{th} glutamate or a corresponding position can also be with glutamate. Substitution in the 154^{th} glutamate or a corresponding position can be with asparagine. Substitution in the 154^{th} glutamate or a corresponding position can also be with glutamine.

Substitution in the 158^{th} glutamate or a corresponding position can be with aspartate. Substitution in the 158^{th} glutamate or a corresponding position can also be with glutamate. Substitution in the 158^{th} glutamate or a corresponding position can be with asparagine. Substitution in the 158^{th} glutamate or a corresponding position can also be with glutamine.

Substitution in the 242^{nd} aspartate or a corresponding position can be with aspartate. Substitution in the 242^{nd} aspartate or a corresponding position can also be with glutamate. Substitution in the 242^{nd} aspartate or a corresponding position can be with asparagine. Substitution in the 242^{nd} aspartate or a corresponding position can also be with glutamine.

Substitution in the 246^{th} glutamate or a corresponding position can be with aspartate. Substitution in the 246^{th} glutamate or a corresponding position can also be with glutamate. Substitution in the 246^{th} glutamate or a corresponding position can be with asparagine. Substitution in the 246^{th} glutamate or a corresponding position can also be with glutamine.

A high-speed variant ChRmine protein can be produced from a parent ChRmine protein selected from the proteins provided in FIG. 8A and SEQ ID NOs: 1 to 29. In some cases, compared to the parent ChRmine protein or a homolog thereof, the high-speed variant ChRmine protein has a substitution at the histidine residue in the 33^{rd} position of ChRmine protein as shown in FIG. 8A or the corresponding residue in the first transmembrane domain of a homolog of the ChRmine protein. For example, a high-speed variant ChRmine protein has an arginine substitution at the histidine residue in the 33^{rd} position of ChRmine protein as shown in FIG. 8A or the corresponding residue in the first transmembrane domain of a homolog of the ChRmine protein.

A parent ChRmine protein can have a sequence selected from SEQ ID NOs: 1 to 29 or a sequence having at least 80% sequence identity to a sequence selected from SEQ ID NOs: 1 to 29. A sequence having at least 80% sequence identity, at least 85% sequence identity, at least 90% sequence identity, at least 95% sequence identity, or at least 99% sequence identity to a sequence selected from SEQ ID NOs: 1 to 29 can have conservative amino acid substitutions as compared a sequence from which it is derived. For example,

In one embodiment, compared to the parent ChRmine protein, a high-speed variant ChRmine protein has a substitution at the histidine residue in the 33^{rd} position or the corresponding residue in the first transmembrane domain of the parent ChRmine protein. The histidine amino acid can be substituted with any other amino acid, for example, histidine (when the corresponding amino acid is not histidine), arginine or lysine, i.e., a basic amino acid.

For example, substitution at the 33^{rd} histidine or a corresponding position can be with arginine. Substitution at the 33^{rd} histidine or a corresponding position can also be with lysine. When the amino acid corresponding to the 33^{rd} histidine is not histidine, such amino acid can be substituted with histidine.

In certain embodiments, a high-speed variant ChRmine protein has an arginine substitution at the histidine residue in the 33^{rd} position or the corresponding residue in the first transmembrane domain of the parent ChRmine protein. Accordingly, a high-speed variant ChRmine protein can have a sequence of SEQ ID NO: 30 or a sequence having at least 80% sequence identity, least 85% sequence identity, at least 90% sequence identity, at least 95% sequence identity, or at least 99% sequence identity to SEQ ID NO: 30, wherein the variations in the sequence having at least 80% sequence identity, least 85% sequence identity, at least 90% sequence identity, at least 95% sequence identity, or at least 99% sequence identity to SEQ ID NO: 30 exclude the amino acid substitution used to produce the high-speed variant ChRmine protein. In a specific embodiment, a high-speed variant ChRmine protein has the sequence of SEQ ID NO: 30.

Additional embodiments of the disclosure provide a red-shifted variant ChRmine protein having a red-shifted spectrum compared to a parent ChRmine protein, wherein the red-shifted variant ChRmine protein has one or more amino acid substitutions compared to the parent ChRmine protein.

A red-shifted variant ChRmine protein can have one or more amino acid substitutions in the retinal binding pocket (RBP) of the parent ChRmine protein. Certain such amino acid substitutions include substitutions in one or more of: 146^{th} isoleucine or a corresponding position; 174^{th} glycine or a corresponding position; 178^{th} phenylalanine or a corresponding position. Each of these positions can be substituted with any other amino acid. Substitutions at the 146^{th} isoleucine or a corresponding position can be with a serine, cysteine, threonine, or methionine, i.e., a hydroxyl or sulfur/selenium-containing amino acid. Substitutions at the 174^{th} glycine or a corresponding position can be with a serine, cysteine, threonine, or methionine, i.e., a hydroxyl or sulfur/selenium-containing amino acid. Substitutions at the 178^{th} phenylalanine or a corresponding position can be with phenylalanine (when the corresponding amino acid is not phenylalanine), tyrosine, or Tryptophan, i.e., an aromatic amino acid.

For example, substitution at the 146^{th} isoleucine or a corresponding position can be with serine. Substitution at the 146^{th} isoleucine or a corresponding position can also be with cysteine. Substitution at the 146^{th} isoleucine or a corresponding position can be with threonine. Substitution at the 146^{th} isoleucine or a corresponding position can also be with methionine.

Substitution at the 174^{th} glycine or a corresponding position can be with serine. Substitution at the 174^{th} glycine or a corresponding position can also be with cysteine. Substitution at the 174^{th} glycine or a corresponding position can be with threonine. Substitution at the 174^{th} glycine or a corresponding position can also be with methionine.

Substitution at the 178^{th} phenylalanine or a corresponding position can be with tyrosine. Substitution at the 178^{th} phenylalanine or a corresponding position can also be with tryptophan. When the corresponding amino acid at the 178^{th} phenylalanine is not phenylalanine, it can be substituted with phenylalanine.

A red-shifted variant ChRmine protein can be produced from a parent ChRmine protein selected from the proteins provided in FIG. 8A or SEQ ID NOs: 1 to 29. In some cases, compared to the parent ChRmine protein, the red-shifted variant ChRmine protein has one or both of: i) a substitution at the isoleucine residue in the 146^{th} position of ChRmine protein as shown in FIG. 8A or a sequence from SEQ ID NOs: 1 to 29 or the corresponding residue in the fourth transmembrane domain of a homolog of the ChRmine protein; and ii) a substitution at the glycine residue in the 174^{th} position of ChRmine protein as shown in FIG. 8A or the corresponding residue in the fifth transmembrane domain of a homolog of the ChRmine protein. For example, compared to the parent ChRmine protein, a red-shifted variant ChRmine protein has one or both of: i) a methionine substitution at the isoleucine residue in the 146^{th} position of ChRmine protein as shown in FIG. 8A or the corresponding residue in the fourth transmembrane domain of a homolog of the ChRmine protein; and ii) a serine substitution at the glycine residue in the 174^{th} position of ChRmine protein as shown in FIG. 8A or the corresponding residue in the fifth transmembrane domain of a homolog of the ChRmine protein.

A parent ChRmine protein can have a sequence selected from SEQ ID NOs: 1 to 29 or a sequence having at least 80% sequence identity, at least 85% sequence identity, at least 90% sequence identity, at least 95% sequence identity, or at least 99% sequence identity to a sequence selected from SEQ ID NOs: 1 to 29. A sequence having at least 80% sequence identity, at least 85% sequence identity, at least 90% sequence identity, at least 95% sequence identity, or at least 99% sequence identity to a sequence selected from SEQ ID NOs: 1 to 29 can have conservative amino acid substitutions as compared a sequence from which it is derived.

In certain embodiments, a red-shifted variant ChRmine protein has one or both of: i) a substitution at the isoleucine residue in the 146^{th} position or the corresponding residue in the fourth transmembrane domain of the parent ChRmine protein; and ii) a substitution at the glycine residue in the 174^{th} position or the corresponding residue in the fifth transmembrane domain of the parent ChRmine protein. The isoleucine residue in the 146^{th} position or the corresponding residue in the fourth transmembrane domain of the parent ChRmine protein can be substituted with any other amino acid, for example, serine, cysteine, threonine, or methionine, i.e., hydroxyl or sulfur/selenium-containing amino acid. The glycine residue in the 174^{th} position or the corresponding residue in the fifth transmembrane domain of the parent ChRmine protein can be substituted with any other amino acid, for example, serine, cysteine, threonine, or methionine, i.e., hydroxyl or sulfur/selenium-containing amino acid.

For example, the isoleucine residue in the 146^{th} position or the corresponding residue in the fourth transmembrane domain of the parent ChRmine protein can be substituted with serine. The isoleucine residue in the 146^{th} position or the corresponding residue in the fourth transmembrane domain of the parent ChRmine protein can also be substituted with cysteine. The isoleucine residue in the 146^{th} position or the corresponding residue in the fourth transmembrane domain of the parent ChRmine protein can be substituted with threonine. The isoleucine residue in the 146^{th} position or the corresponding residue in the fourth transmembrane domain of the parent ChRmine protein can also be substituted with methionine.

The glycine residue in the 174^{th} position or the corresponding residue in the fifth transmembrane domain of the parent ChRmine protein can be substituted with serine. The glycine residue in the 174^{th} position or the corresponding residue in the fifth transmembrane domain of the parent ChRmine protein can also be substituted with cysteine. The glycine residue in the 174^{th} position or the corresponding residue in the fifth transmembrane domain of the parent ChRmine protein can be substituted with threonine. The glycine residue in the 174^{th} position or the corresponding residue in the fifth transmembrane domain of the parent ChRmine protein can also be substituted with methionine.

Accordingly, a red-shifted variant ChRmine protein can have a sequence of SEQ ID NO: 31 or a sequence having at least 80% sequence identity, at least 85% sequence identity, at least 90% sequence identity, at least 95% sequence identity, or at least 99% sequence identity to SEQ ID NO: 31, wherein the variations in the sequence having at least 80% sequence identity, at least 85% sequence identity, at least 90% sequence identity, at least 95% sequence identity, or at least 99% sequence identity to SEQ ID NO: 31 exclude the amino acid substitution used to produce the red-shifted variant ChRmine protein. In a specific embodiment, a red-shifted variant ChRmine protein has a sequence of SEQ ID NO: 31.

Further embodiments of the disclosure provide a high-speed and red-shifted variant ChRmine protein having faster kinetics and red-shifted spectrum compared to a parent ChRmine protein, wherein the high-speed and red-shifted variant ChRmine protein has one or more amino acid substitutions compared to the parent ChRmine protein.

A high-speed and red-shifted variant ChRmine protein can comprise: i) one or more amino acid substitutions in Schiff base counterion of the parent ChRmine protein or one or more amino acid substitutions that alter the pore electrostatic potential of the parent ChRmine protein, and ii) one or more amino acid substitutions in the retinal binding pocket (RBP) of the parent ChRmine protein. In certain cases, a high-speed and red-shifted variant ChRmine protein comprises: i) one or more amino acid substitutions that alter the pore electrostatic potential of the parent ChRmine protein and ii) one or more amino acid substitutions in the retinal binding pocket (RBP) of the parent ChRmine protein.

The one or more amino acid substitutions that alter the pore electrostatic potential can be at: 33^{rd} histidine or a corresponding position; 92^{nd} aspartate or a corresponding position; 154^{th} glutamate or a corresponding position; 158^{th} glutamate or a corresponding position, 242^{nd} aspartate or a corresponding position, or 246^{th} glutamate or a corresponding position. The 33^{rd} histidine or a corresponding position can be substituted with histidine, when the corresponding amino acid is not histidine, arginine, or lysine. Each of the 92^{nd} aspartate or a corresponding position; 154^{th} glutamate or a corresponding position; 158^{th} glutamate or a corresponding position, 242^{nd} aspartate or a corresponding position, and 246^{th} glutamate or a corresponding position can be independently substituted with aspartate, glutamate, asparagine, or glutamine.

For example, substitution at the 33^{rd} histidine or a corresponding position can be with arginine. Substitution at the 33^{rd} histidine or a corresponding position can also be with lysine. When the amino acid corresponding to the 33^{rd} histidine is not histidine, such amino acid can be substituted with histidine.

Substitution in the 92^{nd} aspartate or a corresponding position can be with aspartate. Substitution in the 92^{nd} aspartate or a corresponding position can also be with glutamate. Substitution in the 92^{nd} aspartate or a corresponding position can be with asparagine. Substitution in the 92^{nd} aspartate or a corresponding position can also be with glutamine.

Substitution in the 154^{th} glutamate or a corresponding position can be with aspartate. Substitution in the 154^{th} glutamate or a corresponding position can also be with glutamate. Substitution in the 154^{th} glutamate or a corresponding position can be with asparagine. Substitution in the 154^{th} glutamate or a corresponding position can also be with glutamine.

Substitution in the 158^{th} glutamate or a corresponding position can be with aspartate. Substitution in the 158^{th} glutamate or a corresponding position can also be with glutamate. Substitution in the 158^{th} glutamate or a corresponding position can be with asparagine. Substitution in the 158^{th} glutamate or a corresponding position can also be with glutamine.

Substitution in the 242^{nd} aspartate or a corresponding position can be with aspartate. Substitution in the 242^{nd} aspartate or a corresponding position can also be with glutamate. Substitution in the 242^{th} aspartate or a corresponding position can be with asparagine. Substitution in the 242^{nd} aspartate or a corresponding position can also be with glutamine.

Substitution in the 246^{th} glutamate or a corresponding position can be with aspartate. Substitution in the 246^{th} glutamate or a corresponding position can also be with glutamate. Substitution in the 246^{th} glutamate or a corresponding position can be with asparagine. Substitution in the 246^{th} glutamate or a corresponding position can also be with glutamine.

The one or more amino acid substitutions in the RBP of the parent ChRmine protein can comprise substitutions in one or more of: 146^{th} isoleucine or a corresponding position; 174^{th} glycine or a corresponding position; 178^{th} phenylalanine or a corresponding position. The substitution at the 146^{th} isoleucine or a corresponding position can be with a serine, cysteine, threonine, or methionine. The substitution at the 174^{th} glycine or a corresponding position can be with a serine, cysteine, threonine, or methionine. The substitution at the 178^{th} phenylalanine or a corresponding position can be with phenylalanine, when the corresponding amino acid is not phenylalanine, tyrosine, or Tryptophan. Any combinations of these substitutions can be produced.

For example, substitution at the 146^{th} isoleucine or a corresponding position can be with serine. Substitution at the 146^{th} isoleucine or a corresponding position can also be with cysteine. Substitution at the 146^{th} isoleucine or a corresponding position can be with threonine. Substitution at the 146^{th} isoleucine or a corresponding position can also be with methionine.

Substitution at the 174^{th} glycine or a corresponding position can be with serine. Substitution at the 174^{th} glycine or a corresponding position can also be with cysteine. Substitution at the 174^{th} glycine or a corresponding position can be with threonine. Substitution at the 174^{th} glycine or a corresponding position can also be with methionine.

Substitution at the 178^{th} phenylalanine or a corresponding position can be with tyrosine. Substitution at the 178^{th} phenylalanine or a corresponding position can also be with tryptophan. When the corresponding amino acid at the 178^{th} phenylalanine is not phenylalanine, it can be substituted with phenylalanine.

A high-speed and red-shifted variant ChRmine can be produced from a parent ChRmine protein selected from the proteins provided in FIG. 8A or a ChRmine protein having a sequence selected from SEQ ID NOs: 1 to 29. In some cases, compared to the parent ChRmine protein, a high-speed and red-shifted variant ChRmine protein has one or more of: i) a substitution at the histidine residue in the 33^{rd} position of ChRmine protein as shown in FIG. 8A or the corresponding residue in the first transmembrane domain of a homolog of the ChRmine protein; ii) a substitution at the isoleucine residue in the 146^{th} position of ChRmine protein as shown in FIG. 8A or the corresponding residue in the fourth transmembrane domain of a homolog of the ChRmine protein; and iii) a substitution at the glycine residue in the 174^{th} position of ChRmine protein as shown in FIG. 8A or the corresponding residue in the fifth transmembrane domain of a homolog of the ChRmine protein. For example, compared to the parent ChRmine protein, a high-speed and red-shifted variant ChRmine protein has: i) an arginine substitution at the histidine residue in the 33^{rd} position of ChRmine protein as shown in FIG. 8A or the corresponding residue in the first transmembrane domain of a homolog of the ChRmine protein; ii) a methionine substitution at the isoleucine residue in the 146^{th} position of ChRmine protein as shown in FIG. 8A or the corresponding residue in the fourth transmembrane domain of a homolog of the ChRmine protein; and iii) a serine substitution at the glycine residue in the 174^{th} position of ChRmine protein as shown in FIG. 8A or the corresponding residue in the fifth transmembrane domain of a homolog of the ChRmine protein.

A parent ChRmine protein can have a sequence selected from SEQ ID NOs: 1 to 29 or a sequence having at least 80% sequence identity to a sequence selected from SEQ ID NOs: 1 to 29. A sequence having at least 80% sequence identity to a sequence selected from SEQ ID NOs: 1 to 29 can have conservative amino acid substitutions as compared a sequence from which it is derived.

In certain embodiments, a high-speed and red-shifted variant ChRmine protein has one or more of: i) a substitution at the histidine residue in the 33^{rd} position or the corresponding residue in the first transmembrane domain of the parent ChRmine protein, ii) a substitution at the isoleucine residue in the 146^{th} position or the corresponding residue in the fourth transmembrane domain of the parent ChRmine protein; and iii) a substitution at the glycine residue in the 174^{th} position or the corresponding residue in the fifth transmembrane domain of the parent ChRmine protein. The histidine amino acid in the 33^{rd} position or the corresponding residue in the first transmembrane domain of the parent ChRmine protein can be substituted with any other amino acid, for example, histidine (when the corresponding amino acid is not histidine), arginine or lysine, i.e., a basic amino acid. The isoleucine residue in the 146^{th} position or the corresponding residue in the fourth transmembrane domain of the parent ChRmine protein can be substituted with any other amino acid, for example, serine, cysteine, threonine, or methionine, i.e., hydroxyl or sulfur/selenium-containing amino acid. The glycine residue in the 174^{th} position or the corresponding residue in the fifth transmembrane domain of the parent ChRmine protein can be substituted with any other amino acid, for example, serine, cysteine, threonine, or methionine, i.e., hydroxyl or sulfur/selenium-containing amino acid.

For example, substitution at the 33^{rd} histidine or a corresponding position can be with arginine. Substitution at the 33^{rd} histidine or a corresponding position can also be with lysine. When the amino acid corresponding to the 33^{rd} histidine is not histidine, such amino acid can be substituted with histidine.

Also, substitution at the 146^{th} isoleucine or a corresponding position can be with serine. Substitution at the 146^{th} isoleucine or a corresponding position can also be with cysteine. Substitution at the 146^{th} isoleucine or a corresponding position can be with threonine. Substitution at the 146^{th} isoleucine or a corresponding position can also be with methionine.

Substitution at the 174^{th} glycine or a corresponding position can be with serine. Substitution at the 174^{th} glycine or a corresponding position can also be with cysteine. Substitution at the 174^{th} glycine or a corresponding position can be with threonine. Substitution at the 174^{th} glycine or a corresponding position can also be with methionine.

Accordingly, a high-speed and red-shifted variant ChRmine protein can have a sequence of SEQ ID NO: 32 or a sequence having at least 80% sequence identity, at least 85% sequence identity, at least 90% sequence identity, at least 95% sequence identity, or at least 99% sequence identity to SEQ ID NO: 32, wherein the variations in the sequence having at least 80% sequence identity, at least 85% sequence identity, at least 90% sequence identity, at least 95% sequence identity, or at least 99% sequence identity to SEQ ID NO: 32 exclude the amino acid substitution used to produce the high-speed and red-shifted variant ChRmine protein. In a specific embodiment, a high-speed and red-shifted variant ChRmine protein has a sequence of SEQ ID NO: 32. Further embodiments of the disclosure provide a nucleic acid encoding for a variant ChRmine protein disclosed herein. Based on the sequence of a variant ChRmine protein and known codon usage, a person of ordinary skill in the art can design a nucleic acid encoding a specific variant ChRmine protein. A nucleic acid can be optimized for expression in a particular cell, for example, a mammalian cell or an insect cell. Methods of such codon-optimization are well-known in the art and are within the purview of this disclosure.

The nucleic acid encoding such variant ChRmine protein can be incorporated in an expression cassette, for example, an expression vector, for expressing the variant ChRmine protein in a cell. Non-limiting examples of a cell include a bacterial cell, a fungal cell, an insect cell, a plant cell, or a mammalian cell.

Accordingly, further embodiments of the disclosure provide a genetically modified cell comprising a nucleic acid encoding a variant ChRmine protein. Methods of introducing a nucleic acid, for example, a nucleic acid in an expression construct, into a target cell and expressing and purifying the proteins are well-known in the art and such embodiments are within the purview of the disclosure.

### Methods

Optogenetics include genetic modification to the neurons followed by contacting the genetically modified neurons with light. The genetic modification causes the neurons to express light-sensitive ion channels, and contacting the neurons with light activates these channels, influencing the activation of the neuron.

As discussed above, certain embodiments of the disclosure provide variant ChRmine proteins that exhibit faster kinetics and/or red-shifted spectra compared to a parent ChRmine proteins. When used in optogenetic methods, such variant ChRmine proteins provide certain benefits over parent ChRmine proteins.

Accordingly, certain embodiments of the disclosure provide an optogenetic method comprising: genetically modifying a subject to express in the subject's brain cells the variant ChRmine protein disclosed herein, applying stimulating light to the subject's brain, and imaging the subject's brain.

A subject can be a human, a non-human primate, a bovine, a porcine, a feline, or a canine animal.

The details of the optogenetic methods are well known in the art and generally applying such methods using the variant ChRmine proteins disclosed herein is within the purview of the disclosure.

For example, in some cases the method involves electrical stimulation of the brain region using one or more electrodes. These electrodes can be positioned, either temporarily or permanently, at the brain region.

In some cases, the brain region which is genetically modified for an optogenetic method is selected from the group consisting of: hippocampus, septo-hippocampus, anterior cingulate cortex (ACC), basolateral amygdala (BLA), midline thalamus, insulate regions, medial septum, fimbria fornix. In some cases, the brain region is the hippocampus. In some cases, the brain region is the septo-hippocampus. In some cases, the brain region is the ACC. In some case, the brain region is the BLA. In some cases, the brain region is the medial septum. In some cases, the brain region is the fimbria fornix. In some cases, two or more of the listed brain regions are genetically modified.

Additional embodiments of the disclosure provide methods comprising: genetically modifying a subject to express in a cell and/or organ the variant ChRmine protein disclosed herein. The methods can further comprise applying stimulating light to the modified cell and/or organ, and imaging the subject's cell and/or organ. The cell and/or organ can belong to the cardiovascular system, the gastrointestinal system, the urinary system, the respiratory system, the reproductive system, the musculoskeletal system, or the pancreatic/endocrine system.

Notwithstanding the appended claims, the disclosure is also defined by the following Embodiments:

Embodiment 1. A high-speed variant ChRmine protein having faster kinetic properties compared to a parent ChRmine protein, wherein the high-speed variant ChRmine protein has one or more amino acid substitutions compared to the parent ChRmine.

Embodiment 2. The high-speed variant ChRmine protein according to Embodiment 1, comprising one or more amino acid substitutions in the Schiff base counterion of the parent ChRmine protein.

Embodiment 3. The high-speed variant ChRmine protein according to Embodiment 1, comprising one or more amino acid substitutions that alter the pore electrostatic potential of the parent ChRmine protein.

Embodiment 4. The high-speed variant ChRmine protein according to Embodiment 3, wherein the one or more amino acid substitutions that alter the pore electrostatic potential of the parent ChRmine protein are selected from: 33rd histidine or a corresponding position; 92nd aspartate or a corresponding position; 154th glutamate or a corresponding position; 158th glutamate or a corresponding position, 242nd aspartate or a corresponding position, and 246th glutamate or a corresponding position.

Embodiment 5. The high-speed variant ChRmine protein according to Embodiment 4, wherein: the 33rd histidine or a corresponding position is substituted with histidine, when the corresponding amino acid is not histidine, arginine, or lysine.

Embodiment 6. The high-speed variant of ChRmine protein according to Embodiment 4, wherein: each of the 92nd aspartate or a corresponding position, 154th glutamate or a corresponding position, 158th glutamate or a corresponding position, 242nd aspartate or a corresponding position, and 246th glutamate or a corresponding position is substituted independently of each other with aspartate, glutamate, asparagine, or glutamine.

Embodiment 7. The high-speed variant ChRmine protein according to any one of Embodiments 1 to 6, wherein the parent ChRmine protein has a sequence selected from SEQ ID NOs: 1 to 29 or a sequence having at least 80% sequence identity to a sequence selected from SEQ ID NOs: 1 to 29.

Embodiment 8. The high-speed variant ChRmine protein according to any one of Embodiments 1 to 7, wherein, compared to the parent ChRmine protein, the high-speed variant ChRmine protein has a substitution at the histidine residue in the 33rd position or the corresponding residue in the first transmembrane domain of the parent ChRmine protein.

Embodiment 9. The high-speed variant ChRmine protein according to Embodiment 8, wherein the high-speed variant ChRmine protein has an arginine substitution at the histidine residue in the 33^{rd} position or the corresponding residue in the first transmembrane domain of the parent ChRmine protein.

Embodiment 10. The high-speed variant ChRmine protein according to any one of Embodiments 1 to 9, having the sequence of SEQ ID NO: 30 or a sequence having at least 80% sequence identity to SEQ ID NO: 30, wherein the variations in the sequence having at least 80% sequence identity to SEQ ID NO: 30 exclude the amino acid substitution used to produce the high-speed variant ChRmine protein.

Embodiment 11. The high-speed variant ChRmine protein according to any one of Embodiments 1 to 10, having the sequence of SEQ ID NO: 30.

Embodiment 12. A red-shifted variant ChRmine protein having a red-shifted spectrum compared to a parent ChRmine protein, wherein the red-shifted variant ChRmine protein has one or more amino acid substitutions compared to the parent ChRmine protein.

Embodiment 13. The red-shifted variant ChRmine protein according to Embodiment 12, comprising one or more amino acid substitutions in the retinal binding pocket (RBP) of the parent ChRmine protein.

Embodiment 14. The red-shifted variant ChRmine protein according to Embodiment 13, wherein the one or more amino acid substitutions in the RBP of the parent ChRmine protein comprise substitutions in one or more of: 146th isoleucine or a corresponding position; 174th glycine or a corresponding position; 178th phenylalanine or a corresponding position.

Embodiment 15. The red-shifted variant ChRmine protein according to Embodiment 13, wherein: the substitution at the 146th isoleucine or a corresponding position is with a serine, cysteine, threonine, or methionine; the substitution at the 174th glycine or a corresponding position is with a serine, cysteine, threonine, or methionine; or the substitution at the 178th phenylalanine or a corresponding position is with phenylalanine, when the corresponding amino acid is not phenylalanine, tyrosine, or Tryptophan.

Embodiment 16. The red-shifted variant ChRmine protein according to any one of Embodiments 12 to 15, wherein the parent ChRmine protein has a sequence selected from SEQ ID NOs: 1 to 29 or a sequence having at least 80% sequence identity to a sequence selected from SEQ ID NOs: 1 to 29.

Embodiment 17. The red-shifted variant ChRmine protein according to any one of Embodiments 12 to 16, wherein, compared to the parent ChRmine protein, the red-shifted variant ChRmine protein has one or both of: i) a substitution at the isoleucine residue in the 146th position or the corresponding residue in the fourth transmembrane domain of the parent ChRmine protein; and ii) a substitution at the glycine residue in the 174th position or the corresponding residue in the fifth transmembrane domain of the parent ChRmine protein.

Embodiment 18. The red-shifted variant ChRmine protein according to Embodiment 17, wherein, compared to the parent ChRmine protein, the red-shifted variant ChRmine protein has one or both of: i) a methionine substitution at the isoleucine residue in the 146th position or the corresponding residue in the fourth transmembrane domain of the parent ChRmine protein; and ii) a serine substitution at the glycine residue in the 174th position or the corresponding residue in the fifth transmembrane domain of the parent ChRmine protein.

Embodiment 19. The red-shifted variant ChRmine protein according to any one of Embodiments 12 to 18, having the sequence of SEQ ID NO: 31 or a sequence having at least 80% sequence identity to SEQ ID NO: 31, wherein the variations in the sequence having at least 80% sequence identity to SEQ ID NO: 31 exclude the amino acid substitution used to produce the red-shifted variant ChRmine protein.

Embodiment 20. The red-shifted variant ChRmine protein according to any one of Embodiments 12 to 18, having the sequence of SEQ ID NO: 31.

Embodiment 21. A high-speed and red-shifted variant ChRmine protein having faster kinetics and red-shifted spectrum compared to a parent ChRmine protein, wherein the high-speed and red-shifted variant ChRmine protein has one or more amino acid substitutions compared to the parent ChRmine protein.

Embodiment 22. The high-speed and red-shifted variant ChRmine protein according to Embodiment 21, comprising: i) one or more amino acid substitutions in Schiff base counterion of the parent ChRmine protein or one or more amino acid substitutions that alter the pore electrostatic potential of the parent ChRmine protein, and ii) one or more amino acid substitutions in the retinal binding pocket (RBP) of the parent ChRmine protein.

Embodiment 23. The high-speed and red-shifted variant ChRmine protein according to Embodiment 21 or 22, comprising: i) one or more amino acid substitutions that alter the pore electrostatic potential of the parent ChRmine protein and ii) one or more amino acid substitutions in the retinal binding pocket (RBP) of the parent ChRmine protein.

Embodiment 24. The high-speed and red-shifted variant ChRmine protein according to Embodiment 23, wherein the one or more amino acid substitutions that alter the pore electrostatic potential are selected from: 33rd histidine or a corresponding position; 92nd aspartate or a corresponding position; 154th glutamate or a corresponding position; 158th glutamate or a corresponding position, 242nd aspartate or a corresponding position, and 246th glutamate or a corresponding position.

Embodiment 25. The high-speed and red-shifted variant ChRmine protein according to Embodiment 24, wherein: 33rd histidine or a corresponding position is substituted with histidine, when the corresponding amino acid is not histidine, arginine, or lysine.

Embodiment 26. The high-speed and red-shifted variant ChRmine protein according to Embodiment 24, wherein: each of 92nd aspartate or a corresponding position, 154th glutamate or a corresponding position, 158th glutamate or a corresponding position, 242nd aspartate or a corresponding position, and 246th glutamate or a corresponding position is independently substituted with aspartate, glutamate, asparagine, or glutamine.

Embodiment 27. The high-speed and red-shifted variant ChRmine protein according to any one of Embodiments 22 to 26, wherein the one or more amino acid substitutions in the RBP of the parent ChRmine protein comprise substitutions in one or more of: 146th isoleucine or a corresponding position; 174th glycine or a corresponding position; and 178th phenylalanine or a corresponding position.

Embodiment 28. The high-speed and red-shifted variant ChRmine protein according to Embodiment 27, wherein: the substitution at the 146th isoleucine or a corresponding position is with a serine, cysteine, threonine, or methionine; the substitution at the 174th glycine or a corresponding position is with a serine, cysteine, threonine, or methionine; or the substitution at the 178th phenylalanine or a corresponding position is with phenylalanine, when the corresponding amino acid is not phenylalanine, tyrosine, or Tryptophan.

Embodiment 29. The high-speed and red-shifted variant ChRmine protein according to any one of Embodiments 21 to 28, wherein the parent ChRmine protein has a sequence selected from SEQ ID NOs: 1 to 29 or a sequence having at least 80% sequence identity to a sequence selected from SEQ ID NOs: 1 to 29.

Embodiment 30. The high-speed and red-shifted variant ChRmine protein according to any one of Embodiments 21 to 29, wherein, compared to the parent ChRmine protein, the high-speed and red-shifted variant ChRmine protein has one or more of: i) a substitution at the histidine residue in the 33rd position or the corresponding residue in the first transmembrane domain of the parent ChRmine protein; ii) a substitution at the isoleucine residue in the 146th position or the corresponding residue in the fourth transmembrane domain of the parent ChRmine protein; and iii) a substitution at the glycine residue in the 174th position or the corresponding residue in the fifth transmembrane domain of the parent ChRmine protein.

Embodiment 31. The high-speed and red-shifted variant ChRmine protein according to Embodiment 30, wherein, compared to the parent ChRmine protein, the high-speed and red-shifted variant ChRmine protein has one or more of: i) an arginine substitution at the histidine residue in the 33rd position or the corresponding residue in the first transmembrane domain of the parent ChRmine protein; ii) a methionine substitution at the isoleucine residue in the 146th position or the corresponding residue in the fourth transmembrane domain of the parent ChRmine protein; and iii) a serine substitution at the glycine residue in the 174th position or the corresponding residue in the fifth transmembrane domain of the parent ChRmine protein.

Embodiment 32. The high-speed and red-shifted variant ChRmine protein according to any one of Embodiments 21 to 31, having the sequence if SEQ ID NO: 32 or a sequence having at least 80% sequence identity to SEQ ID NO: 32, wherein the variations in the sequence having at least 80% sequence identity to SEQ ID NO: 32 exclude the amino acid substitution used to produce the high-speed and red-shifted variant ChRmine protein.

Embodiment 33. The high-speed and red-shifted variant ChRmine protein according to any one of Embodiments 21 to 31, having the sequence of SEQ ID NO: 32.

Embodiment 34. A nucleic acid encoding for a variant ChRmine protein according to any one of the preceding Embodiments.

Embodiment 35. A genetically modified cell comprising the nucleic acid according to Embodiment 34.

Embodiment 36. An optogenetic method comprising:
genetically modifying a subject to express in the subject's brain cells the variant ChRmine protein according to any one of Embodiments 1 to 33,
applying stimulating light to the subject's brain, and
imaging the subject's brain.

Embodiment 37. The optogenetic method according to Embodiment 36, wherein the subject is a mammal.

Embodiment 38. The optogenetic method according to Embodiment 37, wherein the mammal is a rodent, a primate, a bovine, a porcine, a feline, or a canine.

Embodiment 39. A method comprising: genetically modifying a subject to express in a cell and/or organ the variant ChRmine protein according to any one of Embodiments 1 to 33.

Embodiment 40. The method of Embodiment 39, further comprising applying stimulating light to the modified cell and/or organ, and imaging the subject's cell and/or organ.

Embodiment 41. The method of Embodiment 40, wherein the cell and/or organ can belong to the cardiovascular system, the gastrointestinal system, urinary system, the respiratory system, the reproductive system, the musculoskeletal system, or the pancreatic/endocrine system.

### EXAMPLES

The following examples are put forth to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1—Crystal Structure Determination of Rhodopsin Proteins

Light—a crucial energy source and environmental signal—is typically captured by motile organisms using rhodopsins, which are largely classified into two groups: microbial and animal, both consisting of a seven-transmembrane (7TM) protein (opsin) and a covalently-bound chromophore (retinal). Light absorption induces retinal isomerization followed by the photocycle, a series of photochemical reactions (Zhang et al., 2011, Ernst et al., 2014; Deisseroth and Hegemann, 2017), which in microbial rhodopsins ultimately exerts direct biochemical action (examples include pumps, channels, sensors, and enzymes (Kandori, 2020; Kato, 2021). Targeted expression of these proteins (especially of the channel- and pump-type) in specific cell types, when applied along with precise light delivery, enables causal study of cellular activity in behaving organisms (optogenetics) (Deisseroth, 2015; Kurihara and Sudo, 2015; Deisseroth, 2021).

In optogenetics, cation-conducting channelrhodopsins (cation ChRs or CCRs) are typically used for activation of target cells (Deisseroth and Hegemann, 2017). The initial description of a CCR (CrChR1 from the chlorophyte C. reinhardtii; Nagel et al., 2002) was followed by characterization of variants that were discovered or designed with new functions spanning ion selectivity, photocurrent amplitude, absorption, sensitivity, and speed (Deisseroth and Hegemann, 2017). Natural CCRs include CrChR2 (ChR2 from C. reinhardtii) (Nagel et al., 2003), VChR1 (ChR1 from V. carteri) (Zhang et al., 2008), and Chrimson (from C. noctigama) (Klapoetke et al., 2014); these were initially described chiefly from chlorophyte algae, but identification of ChRs from other species further expanded the toolkit. In 2016-17, a subfamily of microbial rhodopsins was reported from the cryptophyte G. theta (Govorunova et al., 2016; Yamauchi et al., 2017), identified as CCRs but more homologous to archaeal ion-pumps such as H. salinarum bacteriorhodopsin (HsBR). Moreover, unlike chlorophyte CCRs, cryptophyte CCRs share three amino acids on TM3 crucial for outward proton (H⁺) pumping [the DTD motif (Inoue et al., 2013); D85, T89, and D96 in HsBR] and have been referred to as bacteriorhodopsin-like cation ChRs or BCCRs (Sineshchekov et al., 2017) (FIGS. 8A and 8B). ChRmine, a member of this subfamily discovered through structure-guided mining (Marshel et al., 2019), exhibits extremely high current and light sensitivity as well as a markedly red-shifted spectrum; these properties have enabled all-optical interrogation of hundreds of individually-specified single neurons (Marshel et al., 2019) and fully non-invasive fast control of deep brain circuitry (Chen et al., 2021).

### Experimental Methods and Results

A high-resolution structure for this family of proteins would facilitate understanding structure-function relationships among pump- and channel-type rhodopsins and designing next-generation optogenetic tools. This previously led to creation of the initial anion-conducting ChRs (ACRs, Berndt et al., 2014, 2016; Kato et al., 2012; Wietek et al., 2014)). To that end, this disclosure provides the cryo-electron microscopy (cryo-EM) structure of ChRmine at 2.0 Å resolution. The information about the structure was also used to create variants with faster speed and greater red-shift while preserving high current and light sensitivity. These variant channelrhodopsins as disclosed herein can be used in optical neuroscience research and for targeted functional analysis in diverse systems.

### Structural Determination

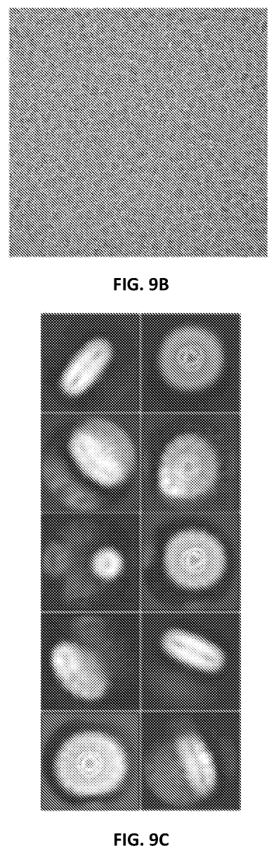

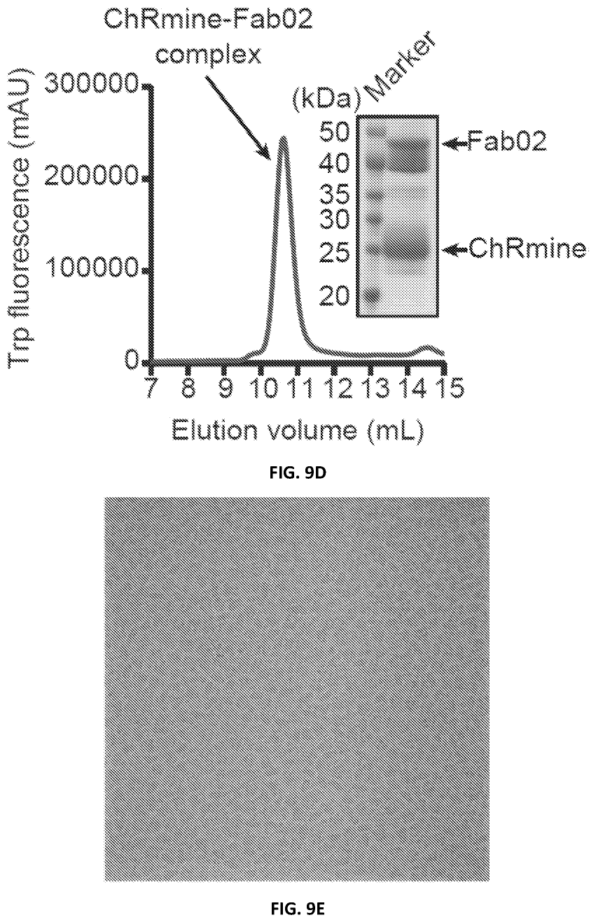

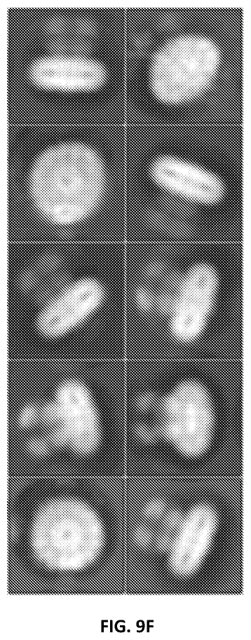

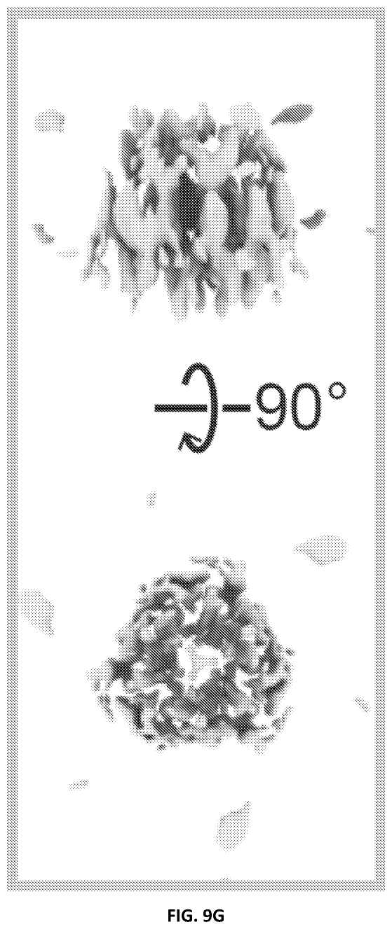

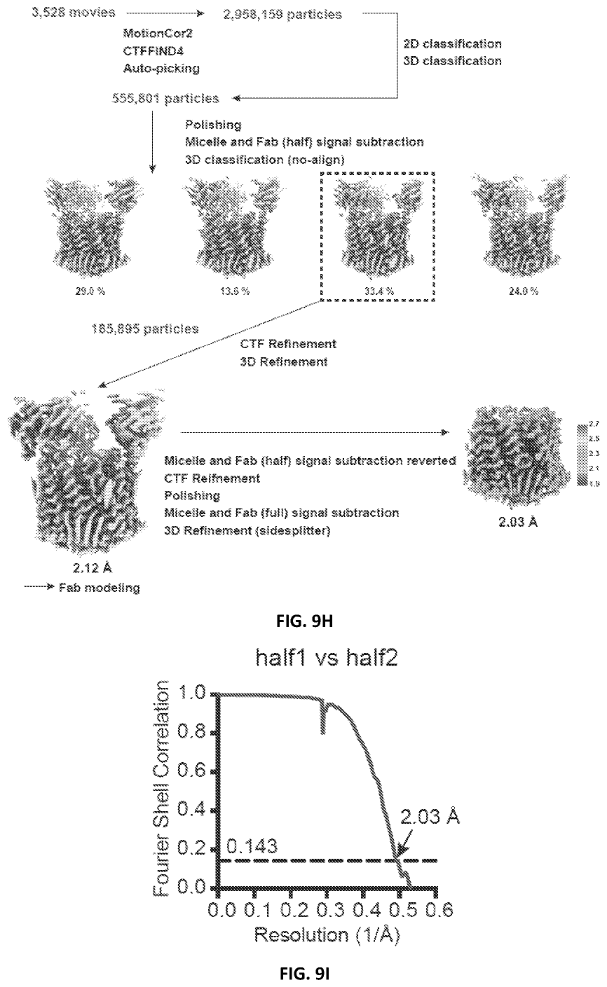

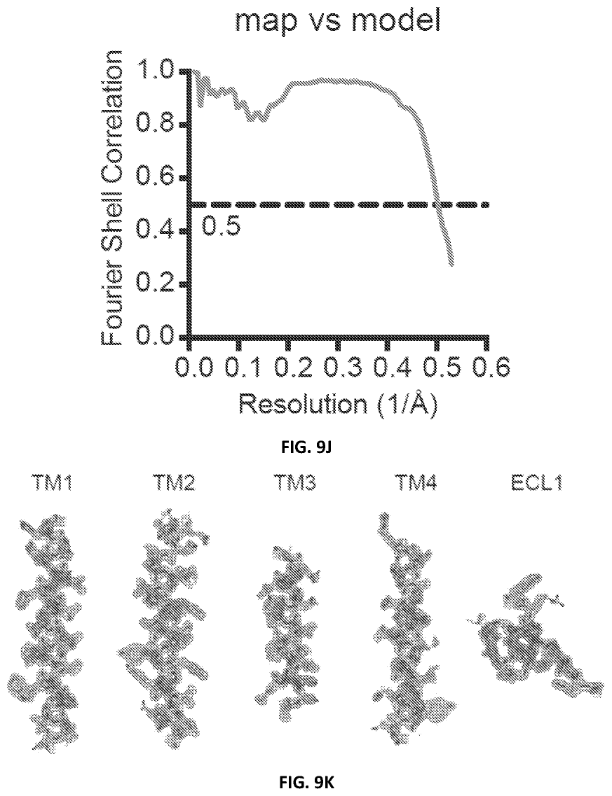

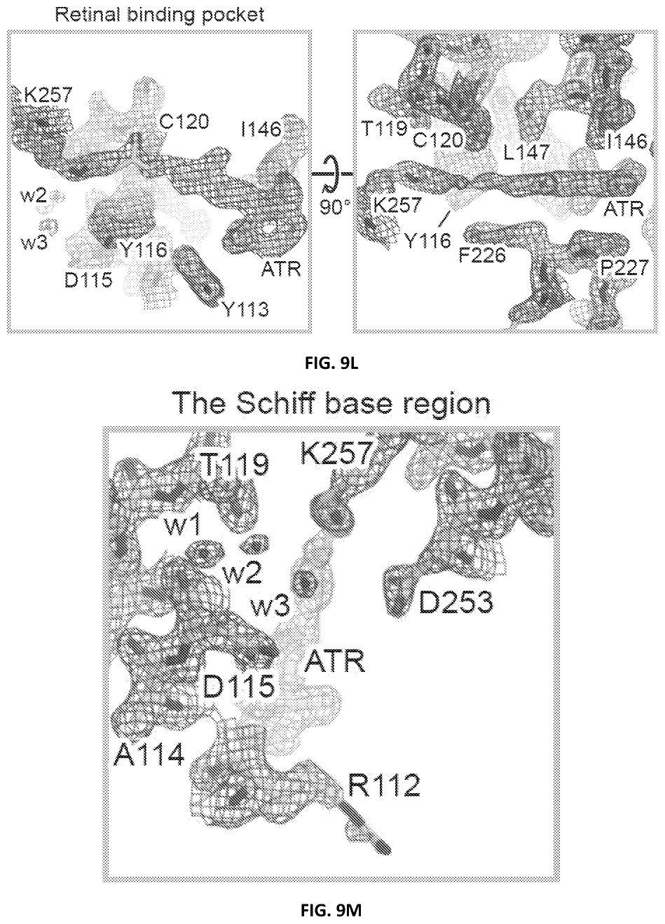

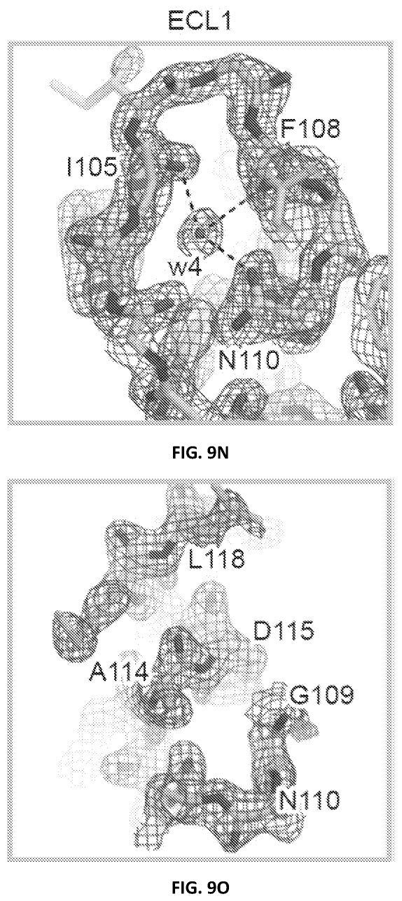

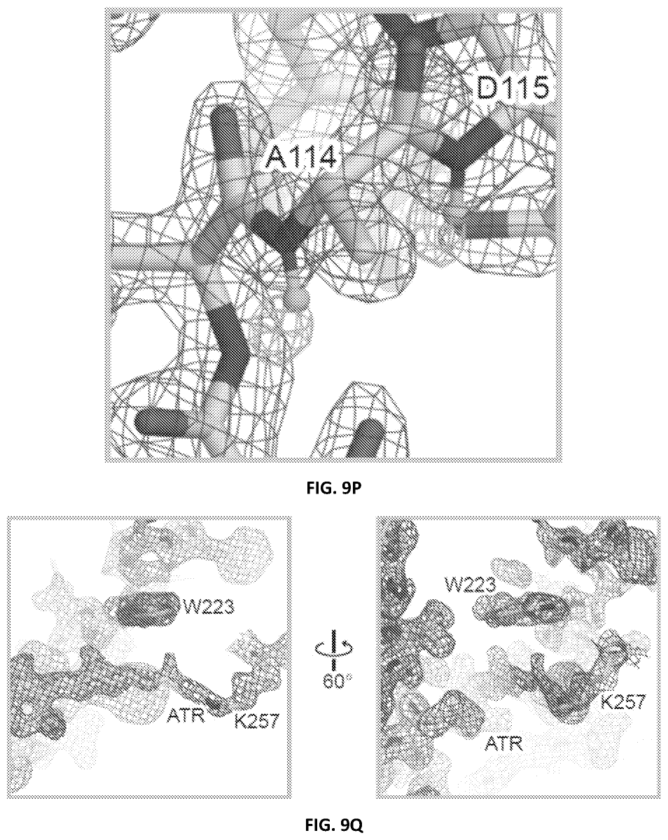

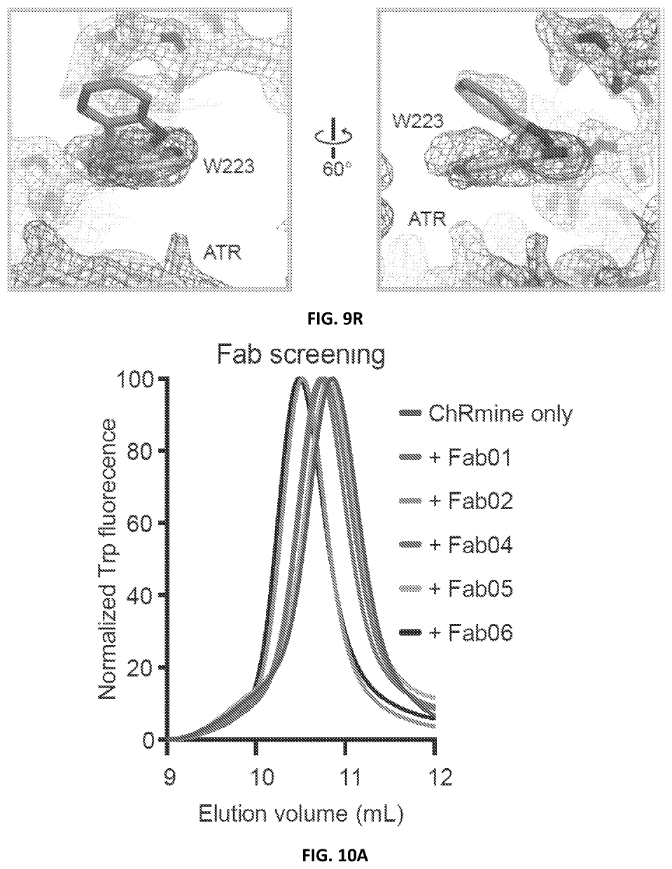

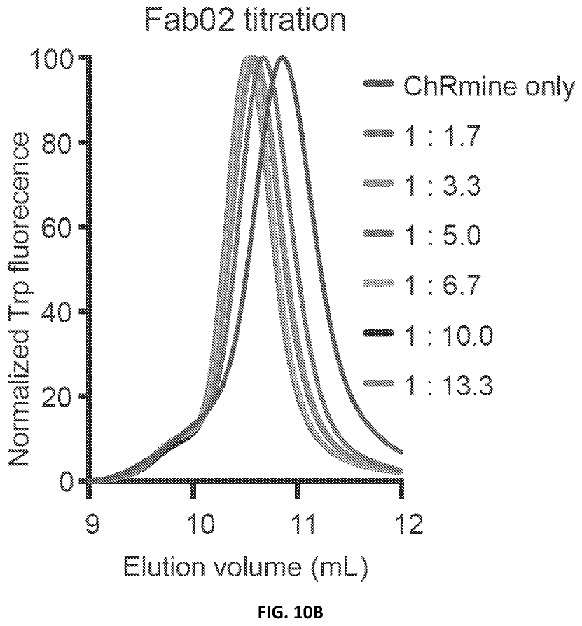

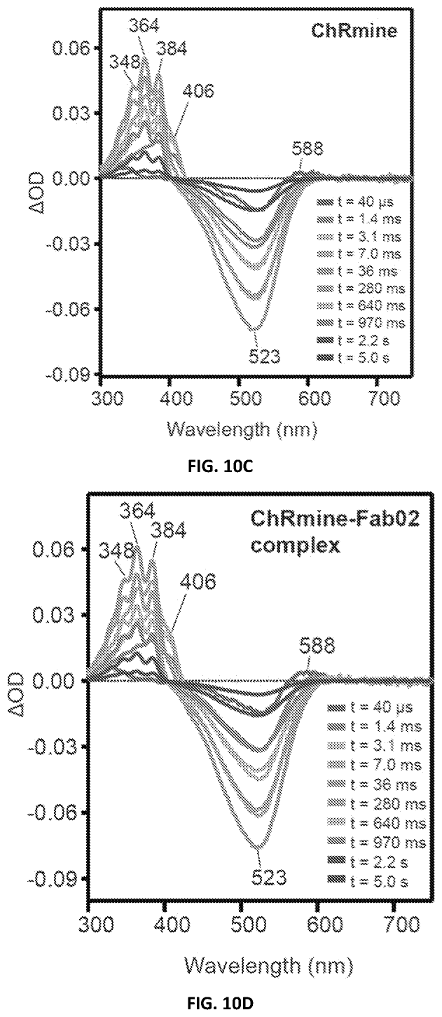

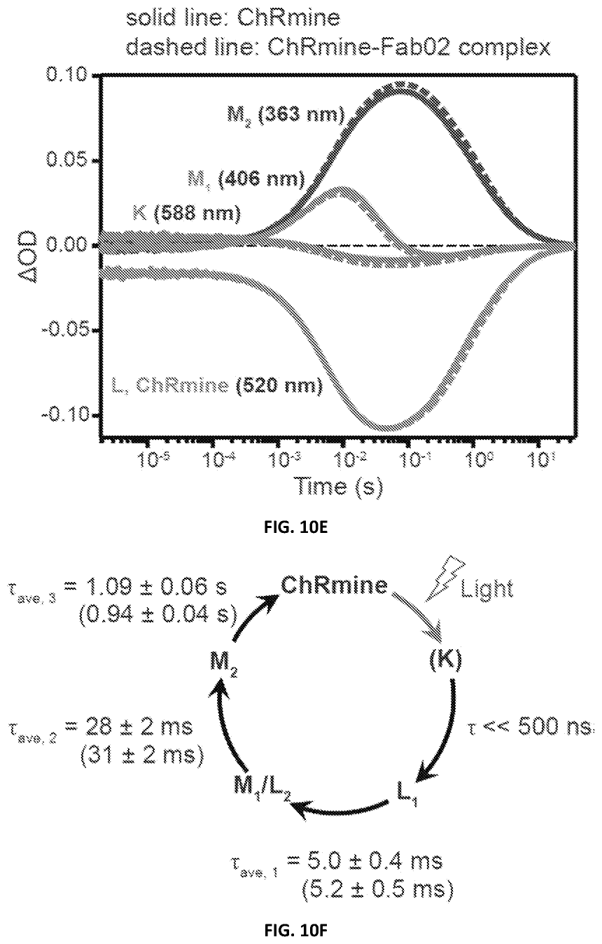

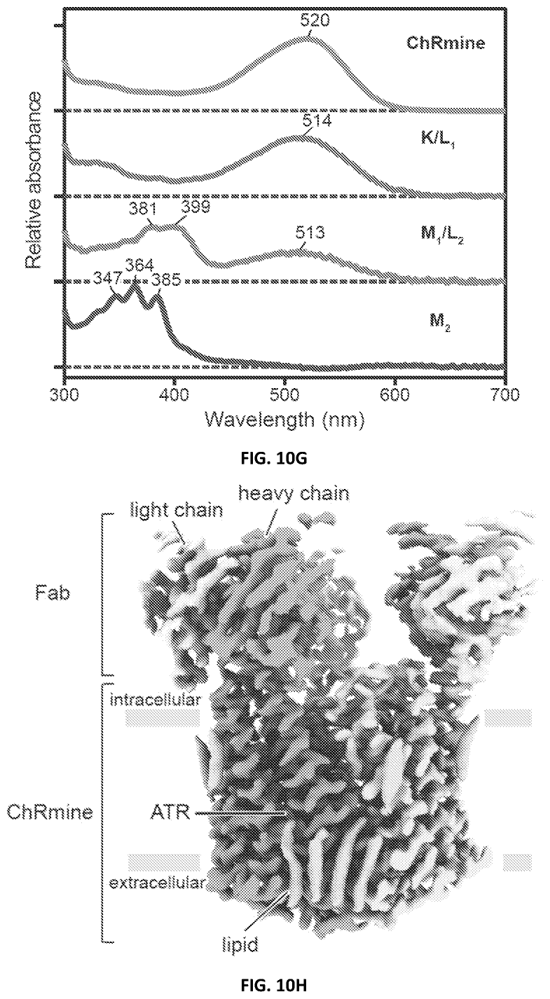

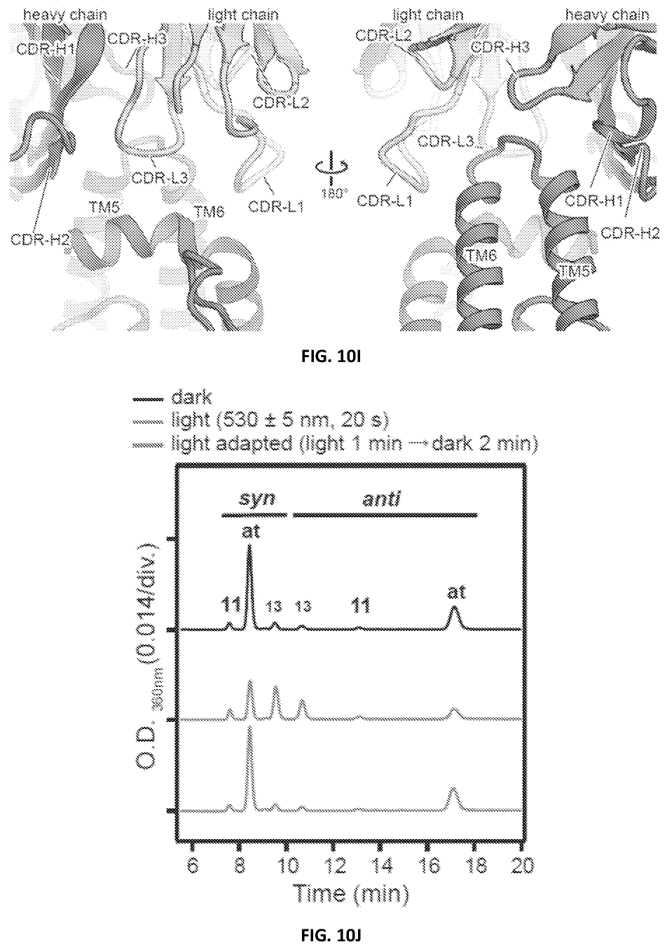

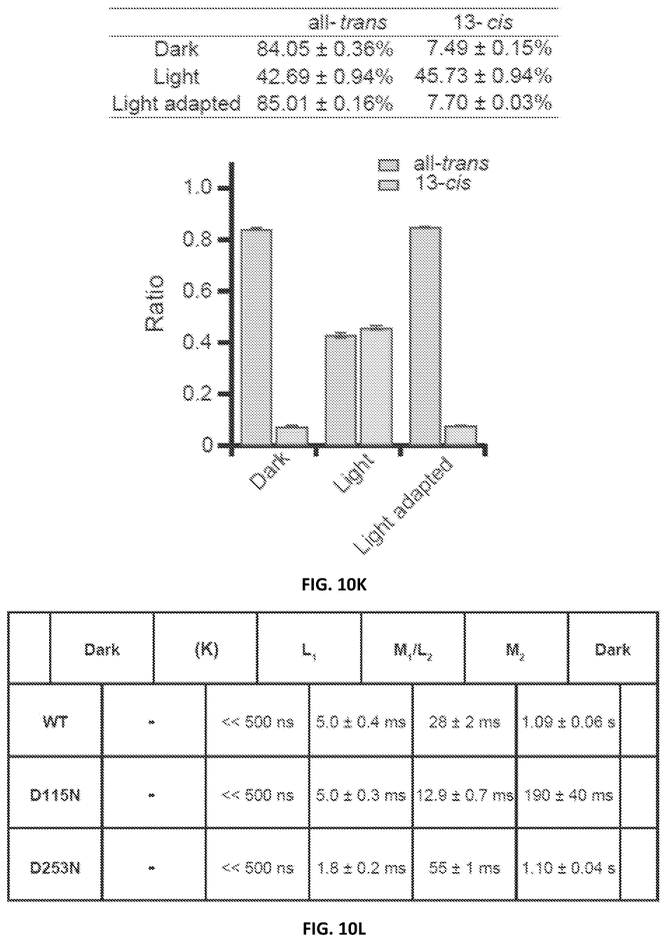

Our initial efforts to crystallize ChRmine yielded low-resolution crystals; we therefore turned to single-particle cryo-EM (FIGS. 9A-9R). The fundamental limitation of single particle cryo-EM is that images of small membrane proteins in detergent micelle have insufficient features for image alignment in data processing. Indeed, due to the compactness of ChRmine (˜35 kDa without extracellular or intracellular domains), particles from an initial cryo-EM dataset were not well-aligned and failed to yield 3D reconstructions (FIGS. 9A-C, 9G). To provide a defined feature for image alignment, we generated a conformation-specific antibody against ChRmine, Fab02 (STAR Methods; FIGS. 10A, 10B). We spectroscopically analyzed the ChRmine-Fab02 complex alongside ChRmine to confirm that Fab02 binding did not affect the photocycle; both ChRmine and ChRmine-Fab02 complexes exhibited K, L₁, L₂, M₁, and M₂ intermediates with similar lifetimes (FIGS. 10C-10G, 10L). Using Fab02, the structure of the ChRmine-Fab02 complex in the dark state was determined at an overall resolution of 2.0 Å (FIGS. 9D-9F, 9H-9J, 10H, 10I; Table 2). The density was of excellent quality, allowing accurate modelling of ChRmine continuously from residues 10 to 279 excluding the disordered N-terminal nine residues and C-terminal 25 residues (FIGS. 9K-9R; STAR methods), and clearly resolved several lipids, water molecules, and the retinal (with the specific all-trans retinal conformer confirmed by HPLC;

FIGS. 9K-9N, 10J, and 10K). Signals of putative hydrogen atoms were observed in the difference (F₀−Fₑ) map (FIGS. 9O, 9P) (Yamashita et al., 2021)—not previously achieved for rhodopsins, and now enabled by this high resolution.

TABLE 2
Cryo-EM data collection and refinement
statistics, Related to STAR Methods

| | ChRmine | ChRmine-Fab02 |
|---|---|---|
| | PDB: 7W9W | EMDB: |
| | EMDB: EMD-32377 | EMD-32378 |
| Data collection and processing | | |
| Microscope | Krios | |
| Magnification | 105,000 | |
| Voltage (kV) | 300 | |
| Electron exposure (e⁻/Å²) | 46 | |
| Defocus range (μm) | 0.8-1.6 | |
| Pixels size at detector | 0.83 | |
| (Å/pixel) | | |
| Symmetry imposed | C3 | |
| Initial particle images (no.) | 2,958,159 | |
| Final particle images (no.) | 185,895 | |
| Map resolution (Å) | 2.02 | 2.12 |
| FSC threshold | 0.143 | 0.143 |
| Map sharpening B factor (Å²) | −35.7 | −38.1 |
| Refinement | | |
| Initial model used (PDB code) | 3UG9 | |
| Model composition | | |
| (monomer) | | |
| Protein residues | 2204 | |
| Retinal | 20 | |
| Lipids | 128 | |
| Water | 48 | |
| B factors (Å²) | | |
| Protein | 58.4 | |
| Retinal | 46.8 | |
| Lipids | 109 | |
| Water | 52.9 | |
| R.m.s. deviations | | |
| Bond lengths (Å) | 0.015 | |
| Bond angles (°) | 2.02 | |
| Validation | | |
| MolProbity score | 0.85 | |
| Clashscore | 1.27 | |
| Poor rotamers (%) | 0.88 | |
| Ramachandran plot | | |
| Favored (%) | 98.51 | |
| Allowed (%) | 1.49 | |
| Outlier (%) | 0 | |

Overall Structure and Comparison with HsBR and C1C2

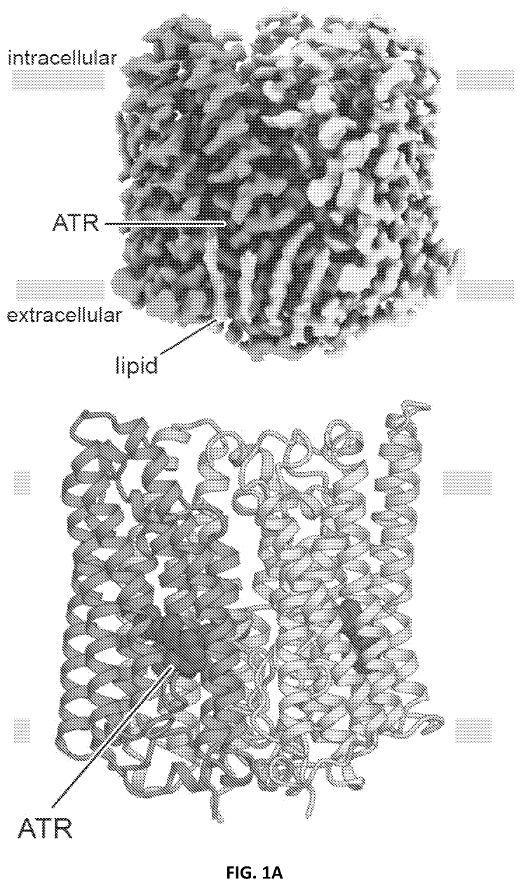

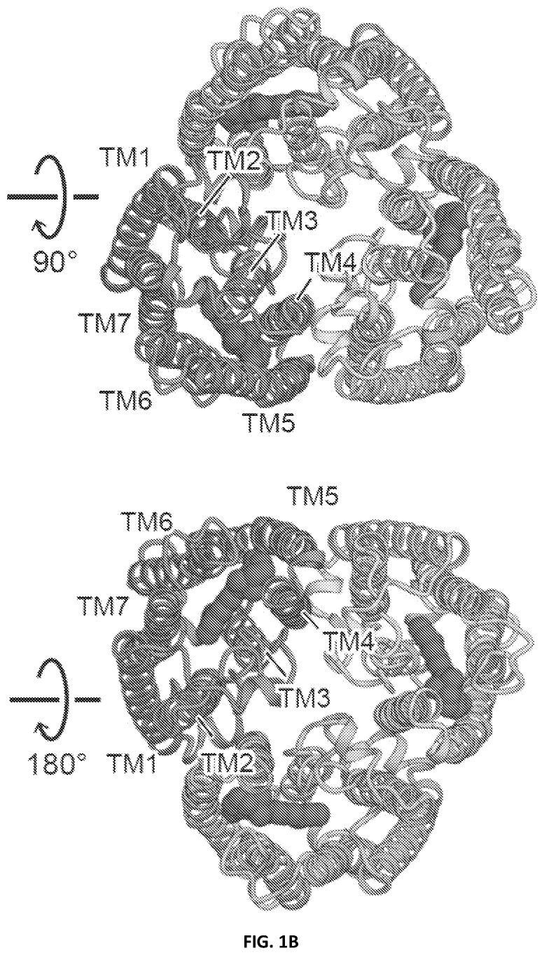

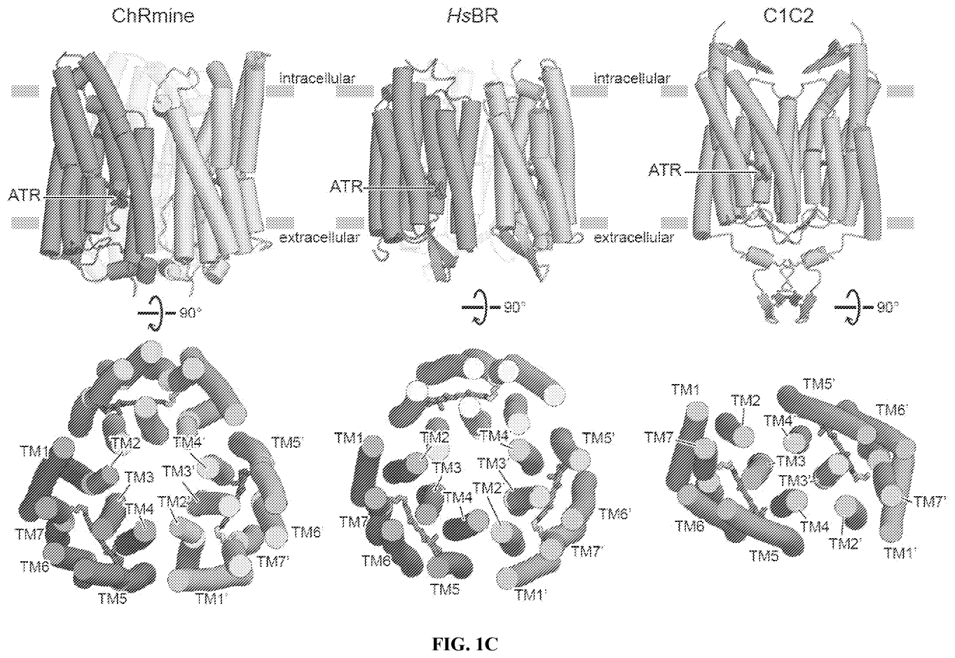

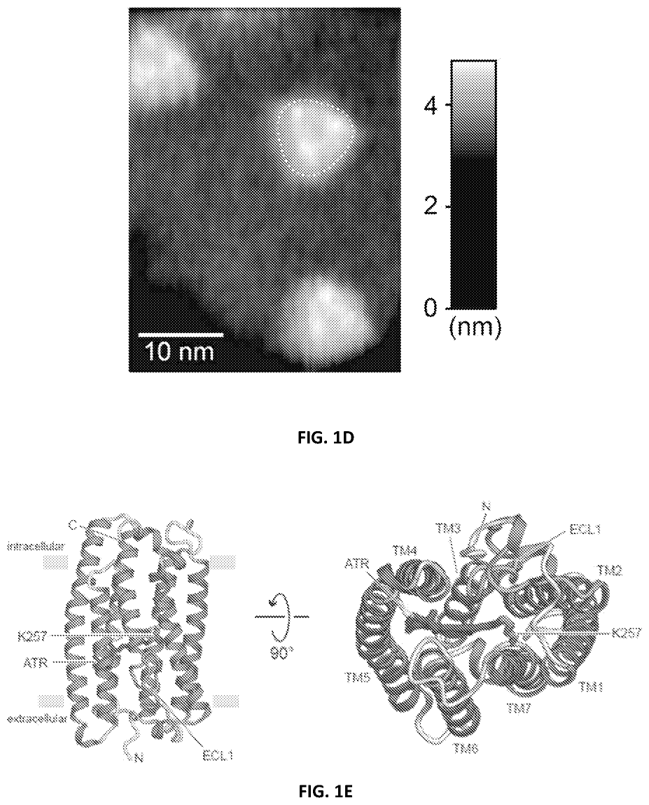

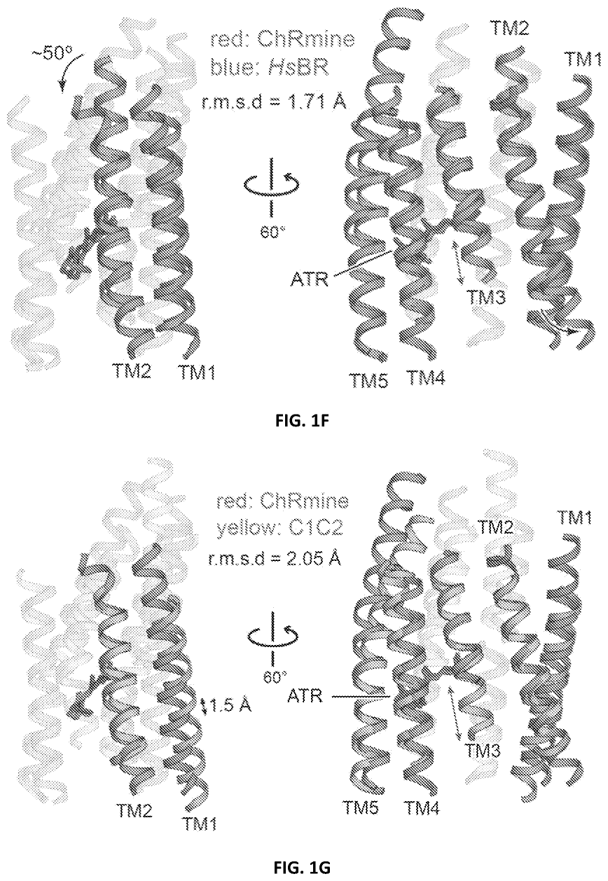

The cryo-EM density map revealed that the quaternary structure of ChRmine is strikingly different from that of other structure-resolved ChRs (Kato et al., 2012) (FIGS. 1A-11B). Instead of the classical ChR dimer, ChRmine forms a trimer, and TM2 interacts with TM4 of adjacent protomers as observed in archaeal ion-pumping rhodopsins including HsBR (Pebay-Peyroula et al., 1997) and HsHR (Kolbe et al., 2000) (FIG. 1C). To confirm this result under more physiological conditions, we reconstituted ChRmine in a lipid bilayer and performed high-speed atomic force microscopy (HS-AFM) which clearly revealed trimeric structure as well (FIG. 1D).

The monomer of ChRmine consists of an extracellular N-terminal domain (residues 10-26), an intracellular C-terminal domain (residues 271-279), and 7 TM domains (within residues 27-270), connected by three intracellular loops (ICL1-3) and three extracellular loops (ECL1-3) (FIG. 1E). TM1-7 adopt a canonical rhodopsin-like topology with a covalently-linked retinal at K257 on TM7, but TM3 markedly diverges from the classical framework, exhibiting an unwound configuration in the middle of the transmembrane region, leading to a long twisting ECL1 (residues 95-115) and a resulting C-shaped structure that is stabilized by an extensive H-bonding network (FIG. 9N).

To explore how ChRmine can be structurally like ion-pumping rhodopsins and yet function as a channel, we compared ChRmine with an archaeal ion-pumping rhodopsin (HsBR), and a chlorophyte CCR (C1C2, the chimera derived from CrChR1 and CrChR2). Consistent with the sequence similarity (FIGS. 8A-8B), ChRmine can be better superimposed onto HsBR; the root-mean-square deviation (r.m.s.d) values of ChRmine vs. HsBR and C1C2 were measured to be 1.83 Å and 2.14 Å, respectively (FIGS. 1F-1G). While previous structural work had revealed that TM1, 2, 3, and 7 of CCRs form the ion-conducting pathway within each monomer and that positioning of TM1/2 structurally distinguishes ChRs from pump-type rhodopsins (Kato et al., 2012), TM1 of ChRmine is positioned more similarly to that of HsBR, and is shifted in its entirety by 1.5 Å in ChRmine relative to C1C2 (FIG. 1G). The overall positioning (and the central region) of TM2 is also similar between ChRmine and HsBR, with the exception that both the intracellular and extracellular regions of TM2 are tilted outward in ChRmine (FIG. 1F); these features in TM2 enlarge the cavity within the monomer and may allow ChRmine to function as a CCR.

### The Schiff Base Region

In all microbial rhodopsins, the retinal is covalently bound to a TM7 lysine to form the protonated Schiff base; this positive charge is stabilized by 1-2 carboxylates on the extracellular side (FIG. 11A). After photon absorption the proton is transferred to a carboxylate, a critical step in operation of most ion-transporting rhodopsins; the carboxylate(s) stabilizing the positive charge and receiving the proton (forming the M intermediate) have been historically termed the Schiff base counterion(s) and proton acceptor, respectively (Zhang et al., 2011). To obtain structural insight into ChRmine channel gating mechanisms and dynamics, we next focused on the counterion and proton acceptor.

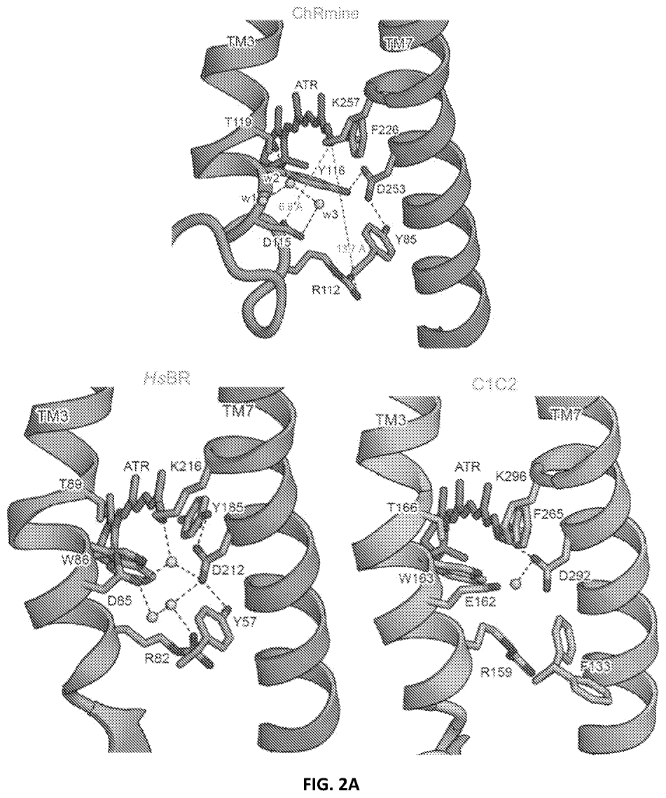

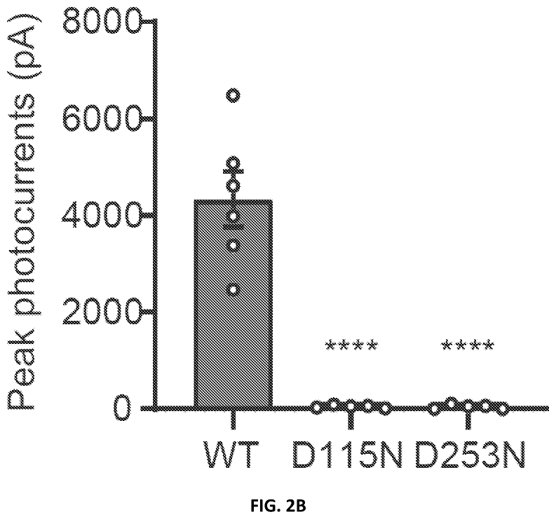

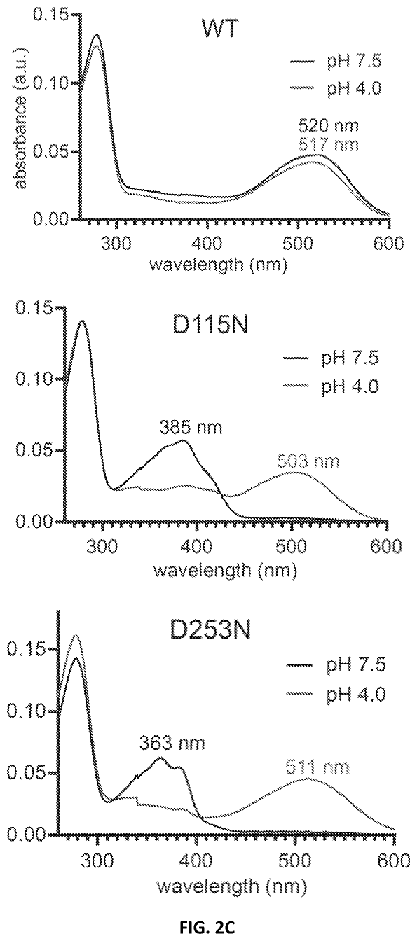

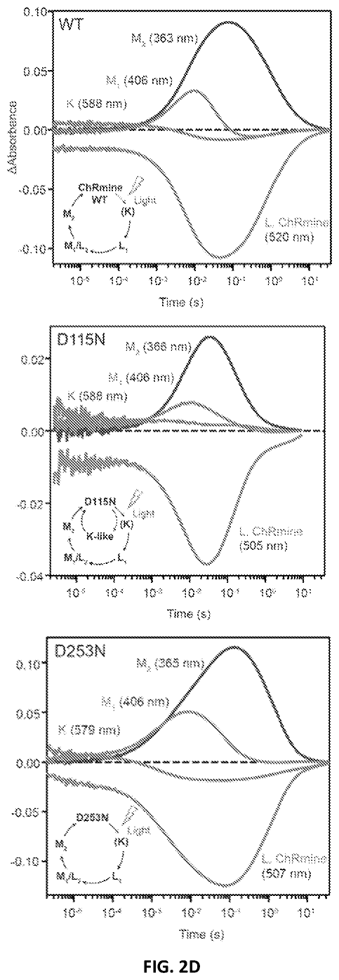

The Schiff base region of ChRmine is strikingly different from that of both types of rhodopsins (HsBR and C1C2; FIG. 2A), even though primary sequence, oligomerization number, and overall monomer structure of ChRmine are like those of HsBR (FIGS. 8A, 8B, 1E-G). In HsBR, the protonated Schiff base nitrogen forms a hydrogen (H)-bond with a water molecule between the counterions, D85 and D212. D212 is fixed by H-bonds with Y57 and Y185 on TM2 and 6, respectively, while D85, which works as the proton acceptor from the Schiff base in the M intermediate (Braiman et al., 1988; Gerwert et al., 1990), interacts with R82 via water molecules (FIG. 2A). In C1C2, the D85, Y57, and Y185 of HsBR are replaced by E162, F133, and F265 respectively; the Schiff base nitrogen H-bonds with D292, which no longer interacts with F133 and F265 (FIG. 2A). D292 acts as the proton acceptor in the M intermediate (Kato et al., 2012; Lorenz-Fonfria et al., 2013), and E162 is dispensable for channel function (Gunaydin et al., 2010; Kato et al., 2012). In contrast, in ChRmine, while the residues corresponding to Y57, R82, D85, and D212 in HsBR are conserved (Y85, R112, D115, D253), R112 and D115 are displaced far from the Schiff base due to the unwinding of TM3 (FIG. 2A); the distances from the Schiff base to R112 and D115 are 13.9 Å and 6.9 Å, respectively, and such long distances have not been observed in microbial rhodopsin structures (FIGS. 11B-11D).

Three water molecules (w1, w2, w3) occupy the space between the Schiff base and D115 created by the unwinding of TM3. Notably, w2 and w3 are well superposed onto the carboxyl oxygens of D85 in HsBR, suggesting that these waters structurally mimic D85 (FIG. 11B) and participate in the counterion complex with D115 and D253. In addition to these structural changes in the TM3 region, the substitutions of Y185 (in HsBR) to F226, and W86 (in HsBR) to Y116, also rearrange the structure in the TM7 region of ChRmine; D253 switches the H-bond from F226 to Y116, and unlike in HsBR, D253 in ChRmine is fixed by two tyrosines (Y85 and Y116) on TM2 and 3 (FIG. 2A).

To explore the function of the counterion and proton acceptor candidates, D115 and D253, we measured photocurrent amplitudes of wild-type (WT), D115N, and D253N ChRmine in HEK293 cells; both D115N and D253N abolished photocurrents (FIGS. 2B,12A). Spectroscopy revealed that these mutants exhibit strikingly blue-shifted absorption spectra (λₘₐₓ at pH 7.5 shifting from 520 nm (WT) to 385 nm (D115N) and 363 nm (D253N)), consistent with loss of function arising from baseline deprotonation of the Schiff base (FIG. 2C). This is the pattern expected if both D115 and D253 work as Schiff base counterions that must be deprotonated at baseline under physiological pH to stabilize the positive charge of the protonated Schiff base, an idea also supported by the pH titration of WT ChRmine in which λₘₐₓ is shifted by decreasing pH, possibly due to protonation of D115 and D253 (FIGS. 11E-11F).

Next, to identify which carboxylate works as a primary proton acceptor in the M intermediate, we performed flash photolysis of D115N and D253N (FIG. 2D). Because rhodopsins with the deprotonated Schiff bases cannot respond to light, photocycles were measured under acidic conditions (pH 4.0) to re-protonate the Schiff base (FIG. 2C). Spectroscopy revealed that both mutants have similar photo-intermediates compared to WT, but only D115N shows additional accumulation of a K-like intermediate (long-lived, up to one second) with lower accumulation of M intermediates (FIG. 2D). Indeed, the M₂ intermediate decay was faster in D115N (TM2=190±40 ms) compared to WT (τ_{M2}=1.09±0.06 s) (FIG. 10L), consistent with D115 operating as a primary proton acceptor. While D253 is located closer to the Schiff base than D115, D253 strongly interacts with Y85 and Y116, which would make it difficult for D253 to receive the proton from the Schiff base. D212 of HsBR similarly interacts with two tyrosine residues (Y57 and Y185) and does not act as the proton acceptor. D115 is located further from the Schiff base but with several waters positioned in between; water rearrangements would allow the proton to transfer from the Schiff base to D115 in the M intermediate.

Ion-Conducting Pore within the Monomer

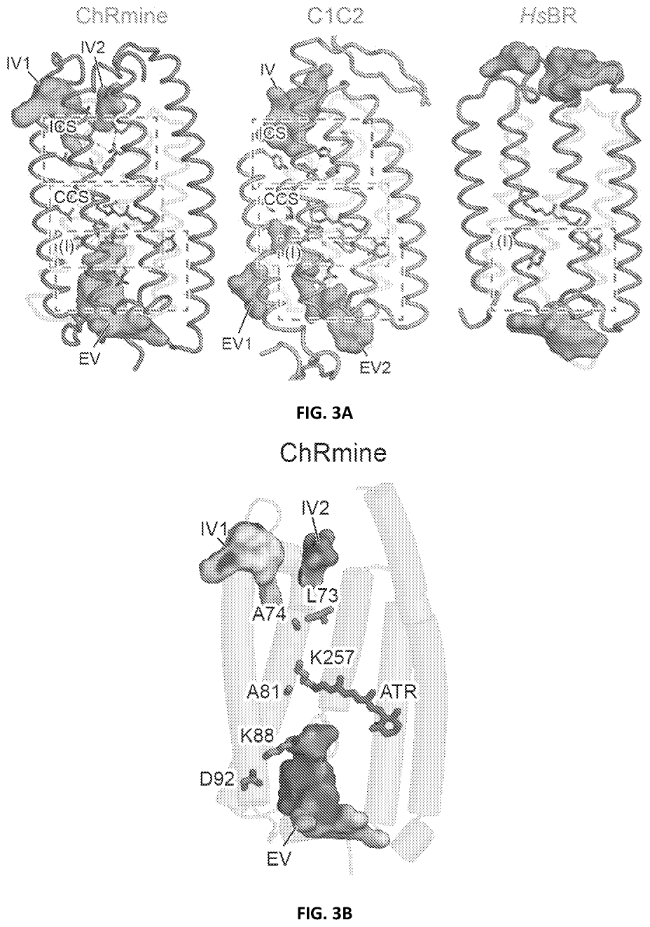

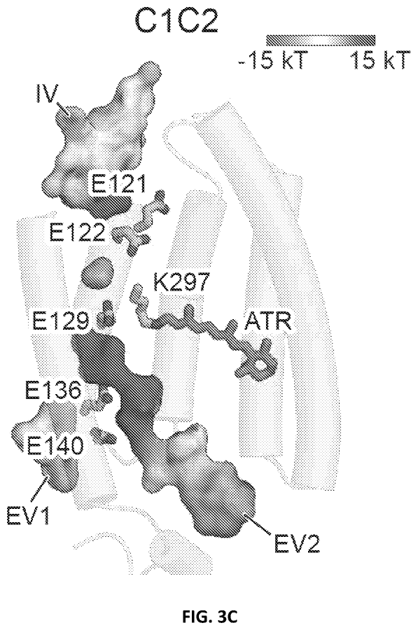

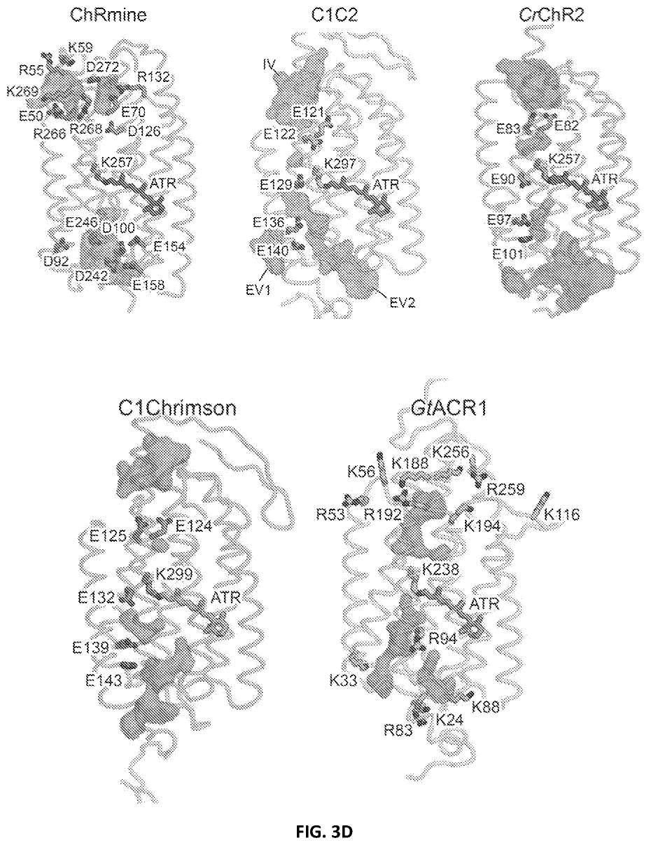

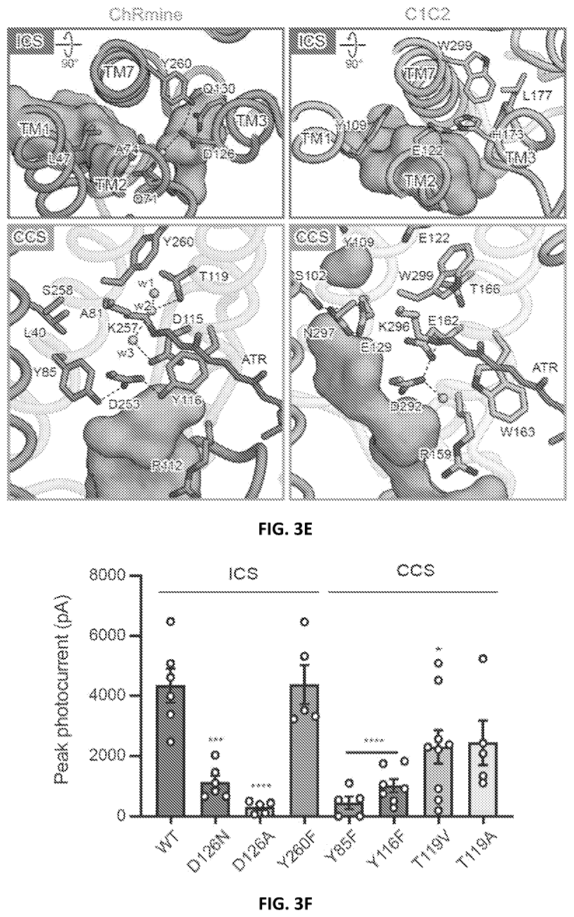

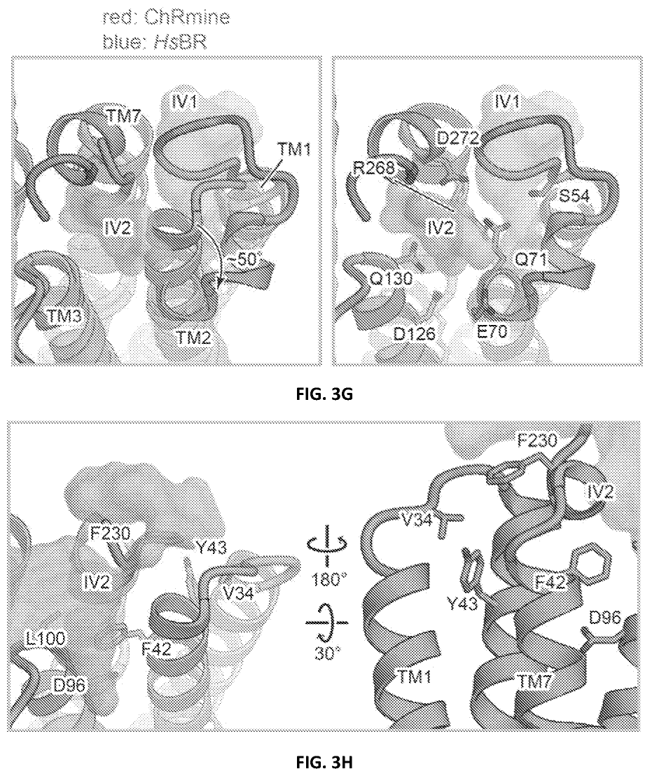

To explore the location and shape of the ion-conducting pathway, we first analyzed the configuration of cavities within the monomer. ChRmine displays markedly larger intracellular and extracellular cavities compared to C1C2 and HsBR (FIG. 3A). As in C1C2, both cavities are mainly formed by TM1, 2, 3, and 7, and occluded by intracellular and central constriction sites (ICS and CCS); however, multiple key differences in the pore pathways of ChRmine and C1C2 were noted. First, while computed electrostatic surface potentials for both ChRmine and C1C2 revealed electronegative pores (FIGS. 3B-3C), the distribution of negatively-charged residues was found to be remarkably different. In C1C2 and several other chlorophyte CCRs, five conserved glutamates (E121, E122, E129, E136, and E140 in C1C2) cooperatively create the electronegative surface potential along the pore, but four of these five residues are substituted with neutral or basic residues in ChRmine (FIGS. 8A, and 3B-3D). Instead, ChRmine displays a distinct set of carboxylates including E50, E70, D100, D126, E154, E158, D242, E246, and D272, to create cavities suitable for anion exclusion and cation selectivity (Berndt and Deisseroth, 2015; Berndt et al., 2014, 2016) (FIG. 3D, top left).

Second, ChRmine exhibits two intracellular vestibules (IV) with distinct electrostatic potentials (FIGS. 3A-3B). Notably, the position of ChRmine IV1 is more similar to the IV of the CCR C1C2, and the position of ChRmine IV2 is more similar to the IV of the ACR GtACR1 (Kato et al., 2012; Kim et al., 2018) (FIGS. 3A-3D), consistent with the fact that ChRmine is phylogenetically closer to GtACR1 than to chlorophyte CCRs including C1C2 (FIG. 8B). The corresponding electrostatic surface potentials favor a role for IV2 as a cation conducting pore in the open state (IV1 and IV2 could further connect to create a larger intracellular cavity in the open state, as for extracellular vestibules in other ChRs (Kato et al., 2018; Takemoto et al., 2015)).

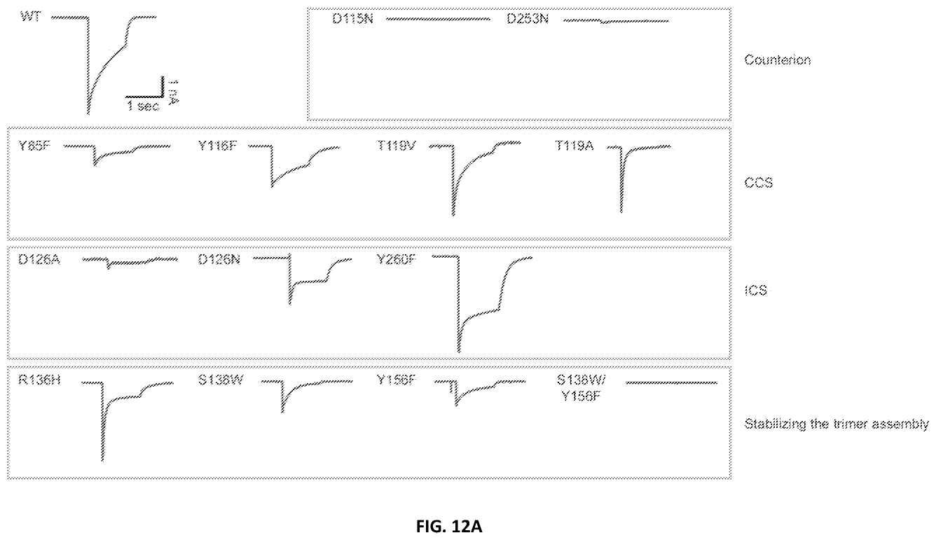

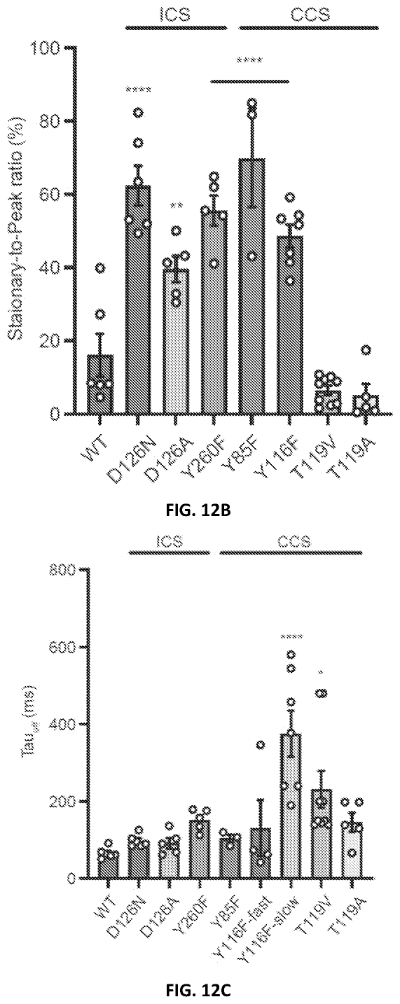

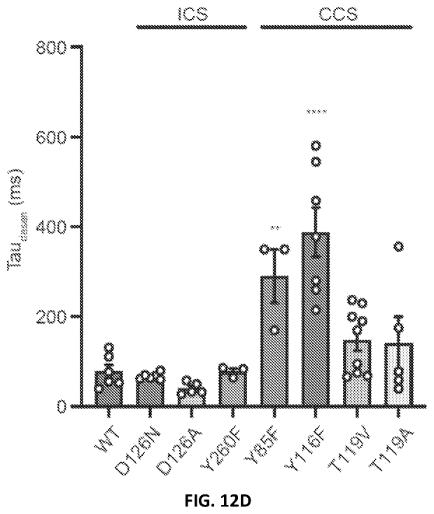

Third, the ICS architecture of ChRmine and C1C2 are different. In C1C2, the ICS is mainly formed by Y109, E122, and H173 (E122 and H173 are H-bonded to each other). In ChRmine, the corresponding residues are L47, A74, and D126, respectively, which participate in the formation of the ICS, but D126 forms a more extensive H-bonding network with Q71, Q130, Y260, and a water (FIG. 3E). While mutation of Y260 does not compromise channel activity, D126 mutants show severely decreased photocurrents, suggesting that the effect of loss of a single H-bond at the ICS is minimal, while even a small change to D126 as the H-bonding network hub significantly affects channel activity (FIGS. 3F, 12).

Fourth, the size and path of the extracellular cavities significantly differ between ChRmine and C1C2. C1C2 has two extracellular vestibules (EV1 and EV2), but ChRmine lacks the vestibule corresponding to EV1, while the volume of ChRmine's sole EV is significantly expanded (due in large part to TM3 unwinding; FIG. 3A). In addition, the EV2 of C1C2 is well-separated from the Schiff base and terminates at the CCS formed by S102, E129, and N297; in contrast, the EV of ChRmine extends prominently to the Schiff base region (FIG. 3E), and ChRmine's three residues corresponding to the CCS of C1C2 (L40, A81, S258) do not form a constriction. Instead, the extensive H-bonding network formed by the counterion complexes (including D115, D253, Y85, Y116, T119, and structured water molecules) occlude the pore and define the ChRmine CCS; the importance of this H-bonding network is supported by loss-of-function electrophysiological properties of Y85F, Y116F, and T119V mutant photocurrents (FIG. 3F).

While ChRmine resembles HsBR in some ways (primary sequence, overall arrangement of the secondary structural elements of the monomer, and quaternary structure of the trimer;

FIGS. 1A-1G and 8A), the size and shape of the cavities within the monomer clearly show higher similarity to those of C1C2, consistent with the cation channel functionality of ChRmine (FIG. 3A). We next sought to understand which structural elements contribute to formation of these large cavities that comprise much of the channel pore in ChRmine, by comparing ChRmine and HsBR in more detail. At least two notable features contribute to this formation of the pore structure. First, as described above, both ends of TM2 are tilted outward in ChRmine; the cytoplasmic end of TM2 is particularly tilted, by about 50 degrees, which significantly enlarges the intracellular cavity (FIGS. 1F and 3G). Furthermore, numerous hydrophilic residues (including 554, E70, Q71, D126, Q130, R268, and D272) face into the pore, which together with the structural waters creates an environment suitable for water and ion conduction. In contrast, in HsBR TM2 remains straight through the end, and 6 of the above 7 hydrophilic residues are replaced by hydrophobic residues, which are tightly packed with no water-accessible cavity (FIGS. 1F, 3A right, and 3H).

In a second major channel-enabling feature, the unwinding of TM3 and resulting long ECL1 contribute to creation of a large extracellular cavity in ChRmine. The helical structure of extracellular TM3 is unfolded beginning at Y116, and the C-shaped structure of ECL1 protrudes to the center of the trimer interface. This contrasts with the ECL1 of HsBR, which forms a β-sheet and is in a position that half-occludes the extracellular pore (FIG. 3I left). In addition to the overall position of ECL1, R82 on TM3 and Y79 on ECL1 protrude into and occlude the extracellular cavity in HsBR (FIG. 3I right). However, Y79 is replaced by G109 in ChRmine, and because of the unfolding of TM3, R112 (R82 in HsBR) and G109 are displaced by 4.0 Å and 6.0 Å, respectively, from the corresponding residues of HsBR. As a result, these residues do not block the cavity in ChRmine (FIG. 3I right).

Notably, the ECL1 of C1C2 also forms a β-sheet structure like HsBR and moderately narrows the entrance of the pore—one of the reasons that the extracellular cavity of C1C2 is smaller than that of ChRmine (FIG. 3I left). Moreover, in C1C2, while the residues corresponding to R82 and Y79 of HsBR are similarly positioned, Y79 is replaced by V156, and R159 (R82 in HsBR) adopts a conformation like that of R112 in ChRmine, facing towards the extracellular solvent rather than parallel to the membrane (FIG. 3I right). The outward-facing Arg conformation observed in ChRmine and C1C2 is conserved in other channel-type rhodopsins including CrChR2, C1Chrimson, and GtACR1, and the parallel Arg conformation observed in HsBR is also conserved in other pump-type rhodopsins such as halorhodopsins (inward Cl⁻ pump-type), KR2 (outward Na⁺ pump-type), and schizorhodopsin (inward H⁺ pump-type) (FIG. 11D; STAR Methods). The Arg in the parallel conformation narrows or blocks the extracellular cavity of the ion-translocating pathway; thus, this conformation would contribute to preventing large ion flux in ion-pumping rhodopsins. Notably, CsR (the outward H⁺-pumping rhodopsin from C. subellipsoidea), also has Arg (R83) in the parallel conformation in the dark state (Fudim et al., 2019), and R83Q mutation or mutation of the adjacent Tyr (Y57K) converts the protein's functionality from H⁺ pump to H⁺ channel (Vogt et al., 2015). Moreover, computational analysis of HsBR with R82Q or Y57K mutation reveals that these mutations significantly change the conformation of R82Q or R82, respectively; most notably, R82 faces outward in the Y57K simulation (Vogt et al., 2015). These results suggest that, as well as the overall size of the monomer cavity, the outward-facing Arg conformation in the dark state is a key structural element defining function of ion-transporting rhodopsins. Interestingly, previous studies have reported that the Arg of some ion-pumping rhodopsins is maintained in the parallel conformation during the photocycle (Kouyama et al., 2015; Kovalev et al., 2020), but the corresponding arginine in HsBR transiently changes from parallel to outward-facing to facilitate proton release to the extracellular solvent (Kuhlbrandt, 2000; Nango et al., 2016). Since ChRs presumably evolved from ion-pumping rhodopsins (Inoue et al., 2015), these studies suggest that mutations accumulating near the arginine of ion-pumping rhodopsins gradually stabilized the outward-facing conformation; these rearrangements enlarged the extracellular cavity, enabling the large ion flux of ChRs.

### Functional Importance of Trimetric Assembly

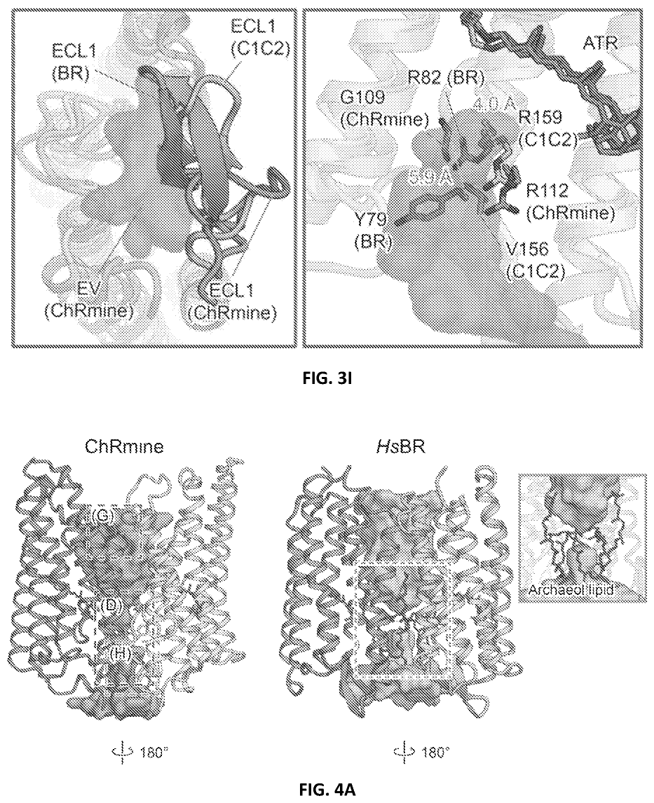

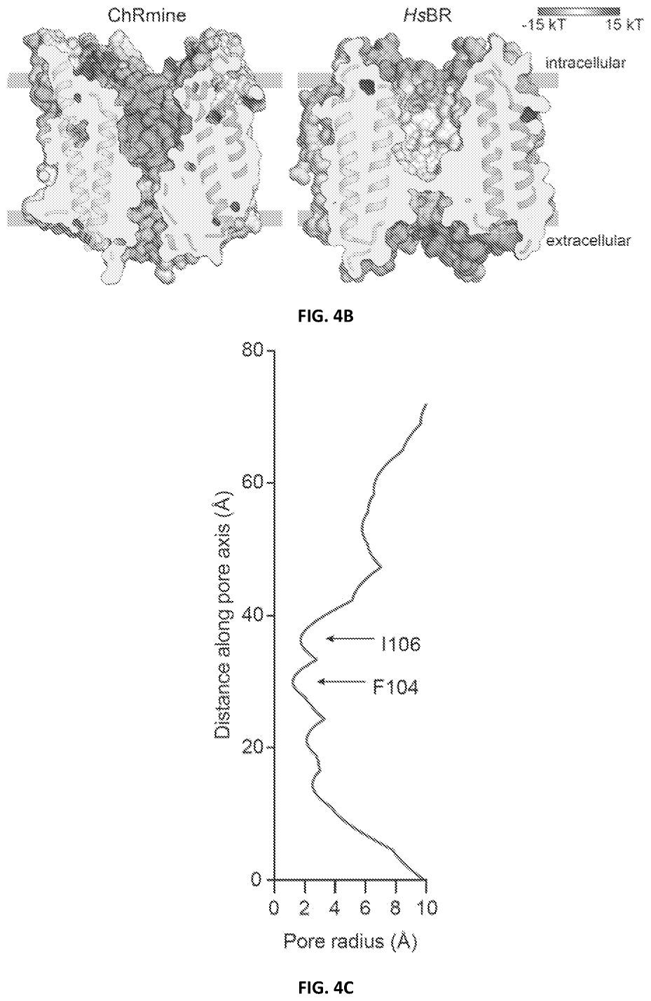

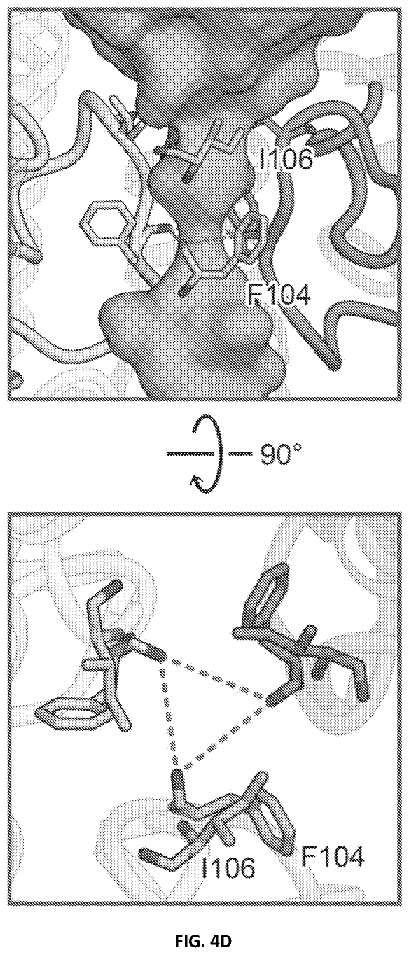

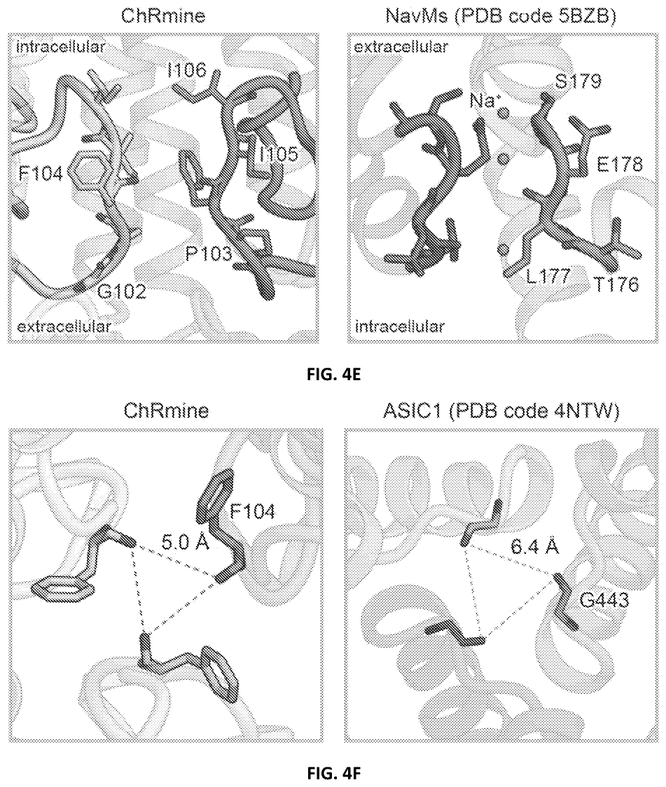

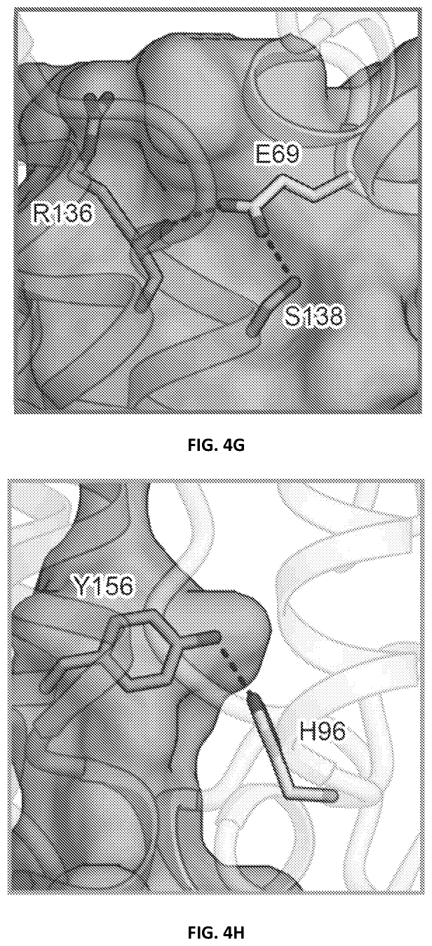

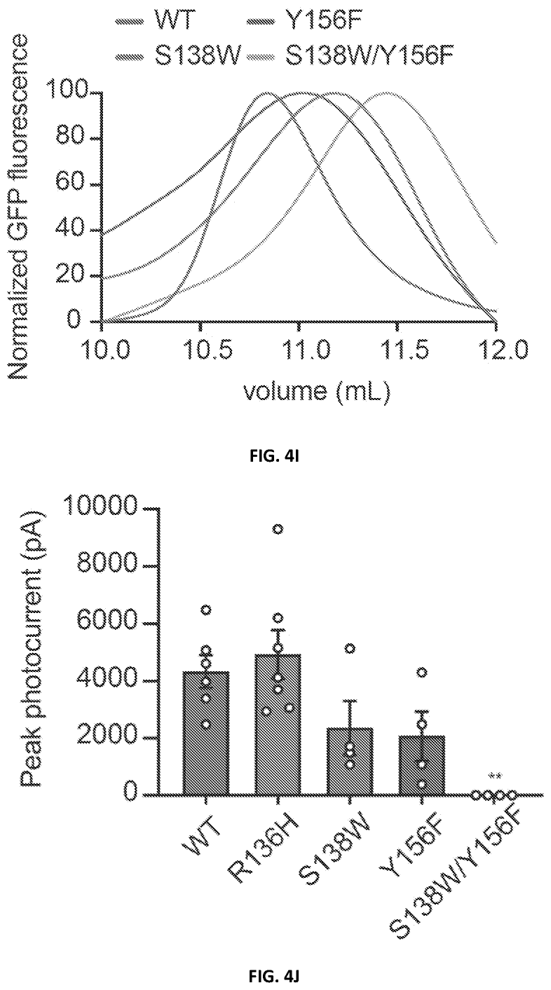

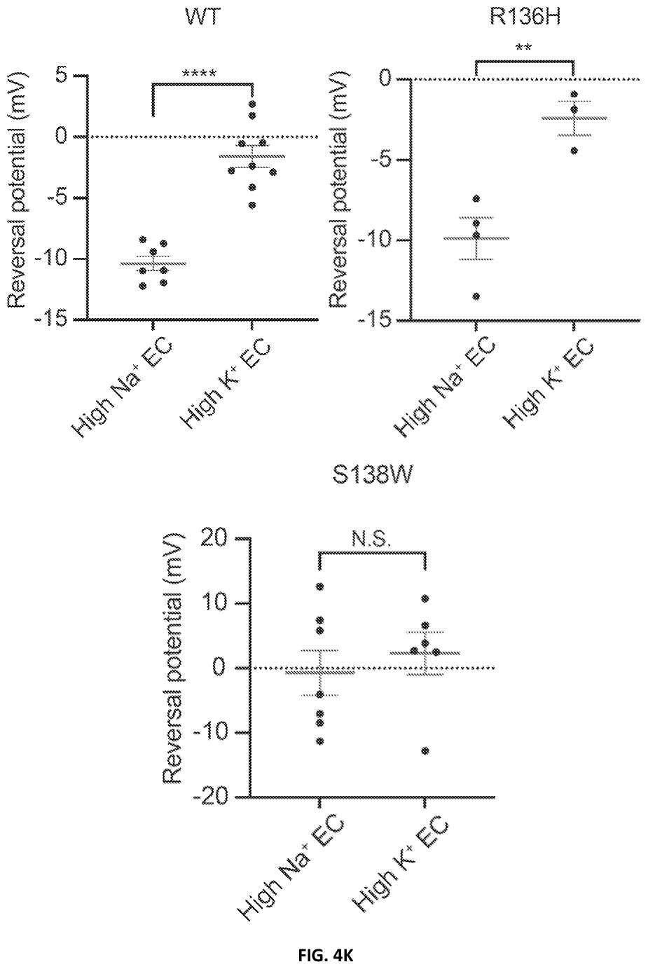

Like HsBR, ChRmine forms a trimer; here we find that ChRmine has an unexpected additional opening at the trimer interface (FIG. 4A left). The corresponding region in HsBR is hydrophobic and filled with several lipid molecules, but for ChRmine this region is relatively hydrophilic and negatively charged (FIGS. 4A right and 4B). The chalice-shaped opening is formed by TM2-4 and ECL1 of the protomer, and the narrowest region is created by ECL1; the main-chain carbonyl oxygens of F104 and 1106 face toward the center of the trimer and form the central constriction (FIGS. 4C and 4D).

ChRmine exhibits three intermolecular H-bond interactions between adjacent protomers: S138 with E69, the main chain amide of R136 with E69, and Y156 with H96 (FIGS. 4E and 4F). To analyze the functional importance of trimeric assembly, we introduced mutations to each of these residues to destabilize the trimer. We used fluorescence-detection size exclusion chromatography (FSEC) (Kawate and Gouaux, 2006) to evaluate oligomerization, and found that S138W or Y156F mutation shifts the equilibrium to favor monomeric states, and S138W/Y156F double mutation almost completely dissociates the trimer into the monomeric state (FIG. 4G). Subsequent electrophysiology revealed that these single and double mutations moderately and severely decrease channel activity, respectively (FIGS. 4H and 12), and the S138W mutation additionally reduces cation selectivity (FIG. 4I). Since S138 and Y156 are unequivocally located far from the canonical ion-conducting pathway within the monomer, and the R136H mutation (which does not affect protomer-protomer interactions) does not change channel activity or cation selectivity, these results support the importance of trimeric assembly for cation conduction, and provide the initial evidence for importance of oligomerization in ChR channel function.

### Computational Analysis of Pore Dynamics

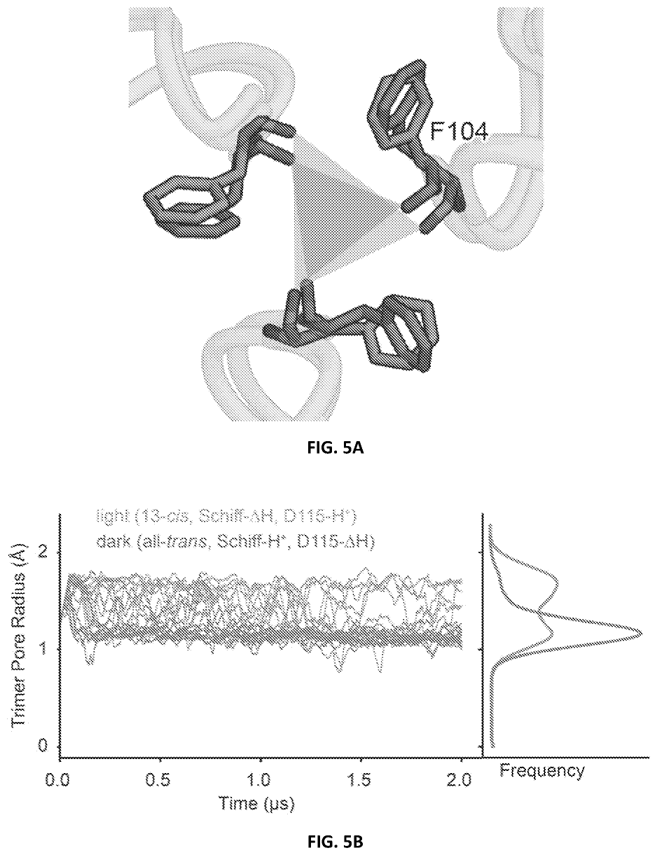

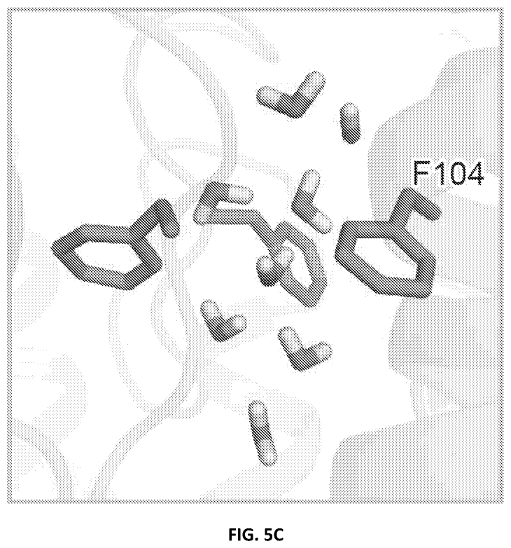

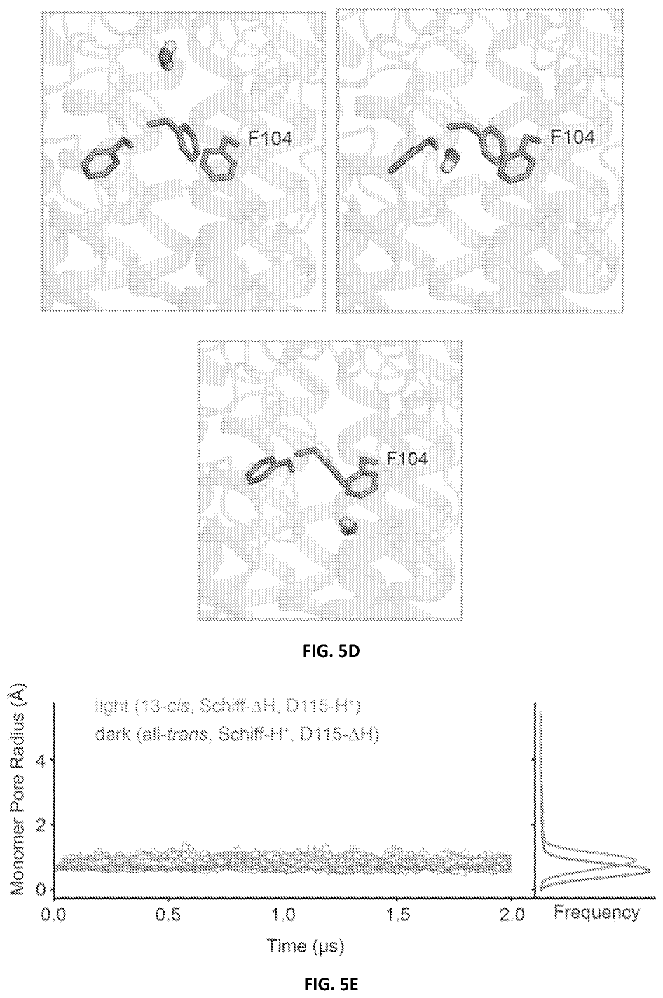

To further test this hypothesis, we performed all-atom molecular dynamics simulations of ChRmine in either the dark state or the M intermediate (light state) (FIGS. 5A-5F). In these simulations of the dark state, the retinal is left in the all-trans configuration and the Schiff base is protonated; in simulations of the light state, the retinal is isomerized to the 13-cis configuration and the Schiff base proton is transferred to the putative proton acceptor D115. Although simulations were not long enough to span complete activation of the channel, key early conformational changes were observed along the pathway toward activation in the light state simulations, in which the trimer pore alternated between a wider, “open” state and a narrower, “closed” state; in contrast, for dark state simulations, the trimer pore remained in the closed state. The trimer pore radius (the radius of the constriction site formed by backbone interactions between the three F104 residues on each of the monomers) was significantly increased in light state simulations compared to dark state simulations (FIGS. 5A and 5B), which sufficed to allow multiple water molecules to pass through the pore (FIGS. 5C and 5D). While the pore did not yet attain a radius sufficient for ion conduction over the timescale of our simulations, these results suggest that the trimer pore is cooperatively coupled to retinal isomerization and support the idea (consistent with the observed ion-selectivity change arising from mutation at the trimer pore (FIG. 4K) that the trimer pore can act as a novel secondary channel, via a structural mechanism not accessible to dimerizing chlorophyte channelrhodopsins or trimerizing pump rhodopsins (Note S2).

### Example 2—Structure-Guided Engineering of ChRmine Variants with New Properties

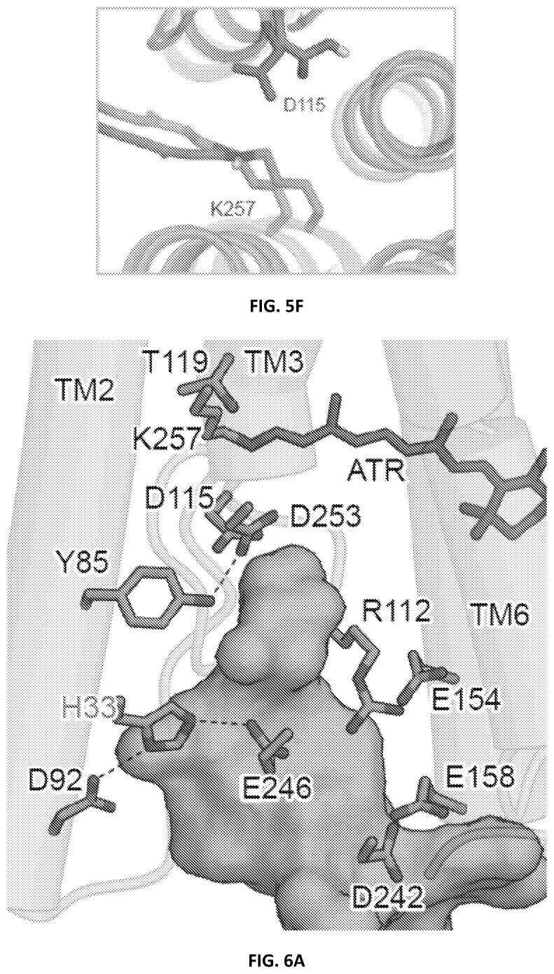

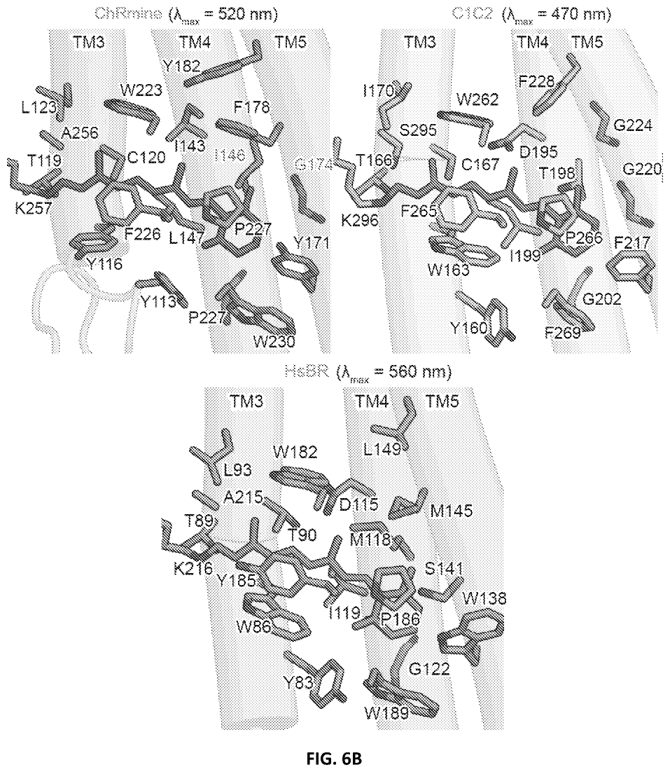

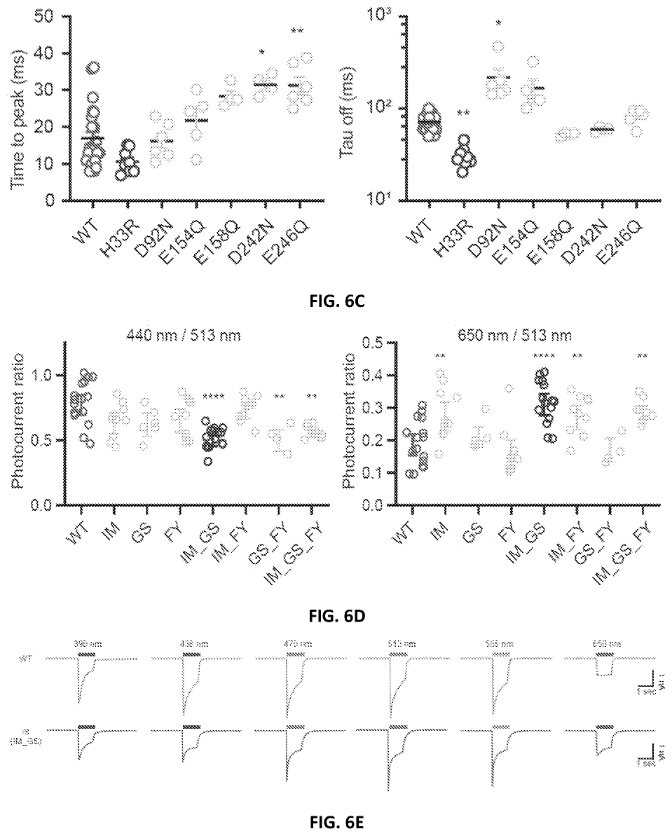

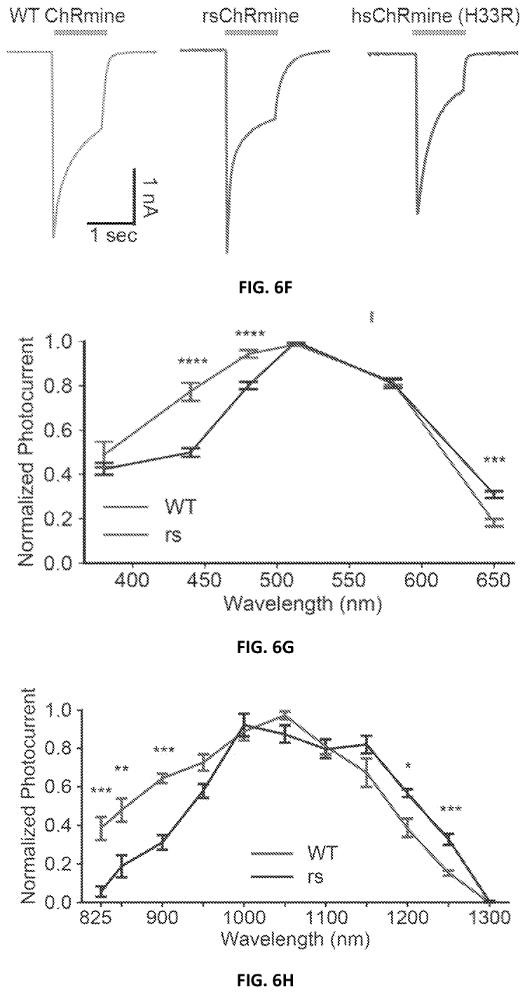

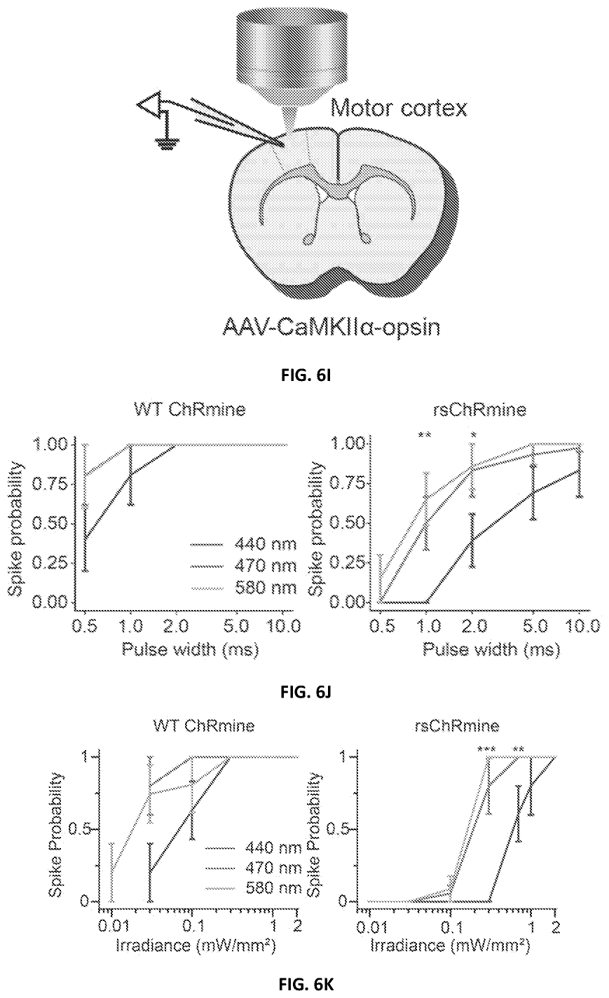

We next sought to enhance the speed and spectral-response of ChRmine for all-optical experiments (FIGS. 6A-6B). We began with speed—noting that if it were possible, a 2-3 fold acceleration of ChRmine kinetics (Marshel et al., 2019) would approach the time constants of principal cells in the brain. One strategy to accelerate closing kinetics would be via mutations to the Schiff base counterion (Gunaydin et al., 2010; Rajasethupathy et al., 2015), but counterion mutations in ChRmine compromise channel function (FIG. 2B).

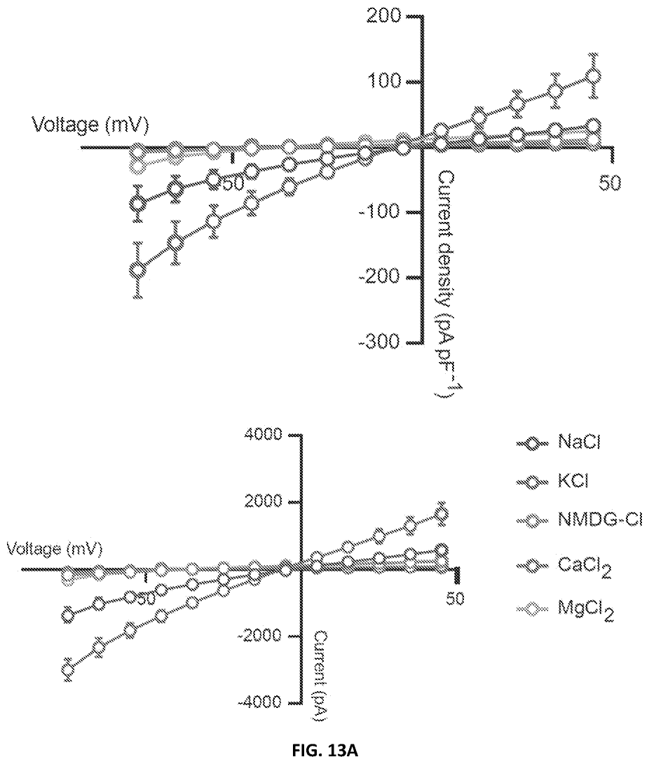

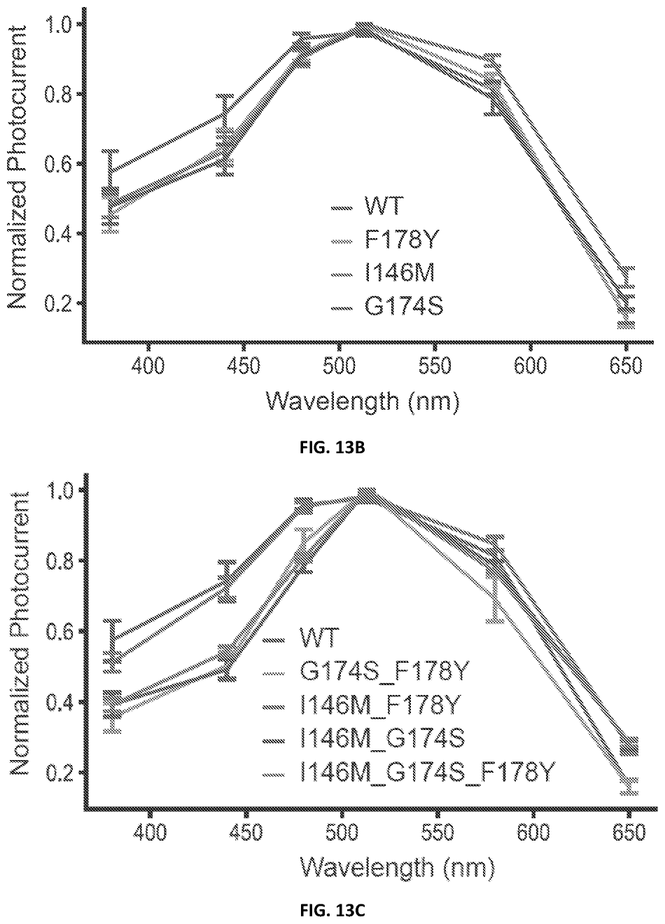

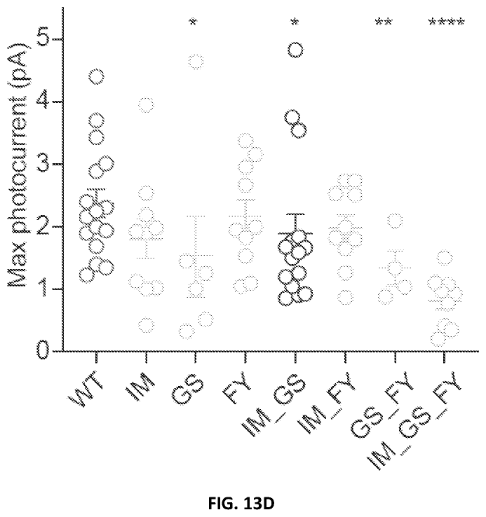

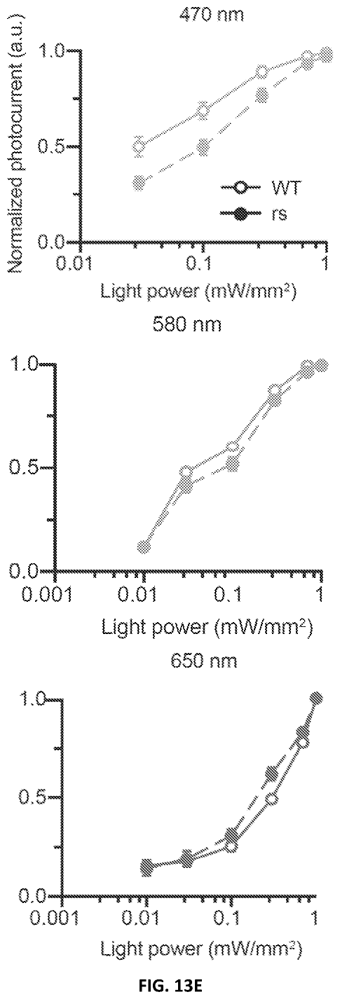

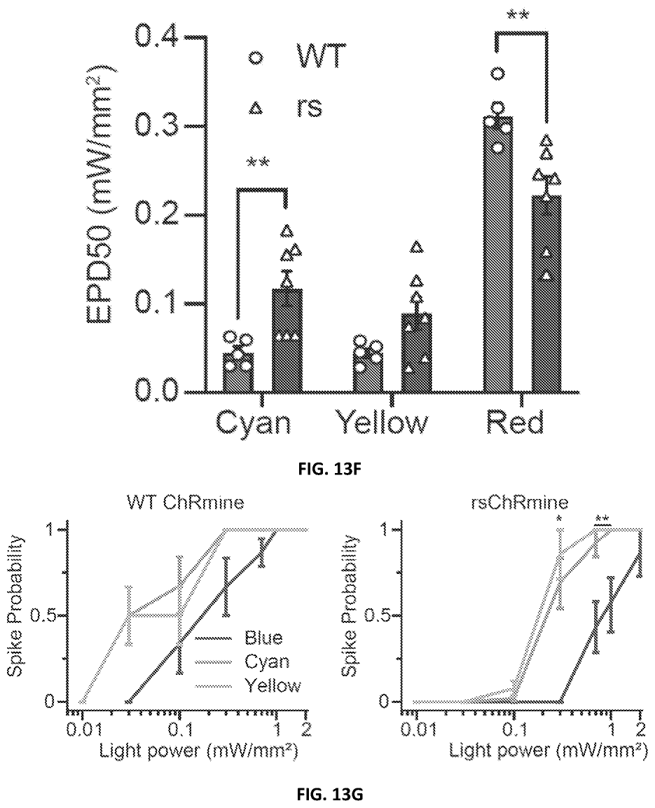

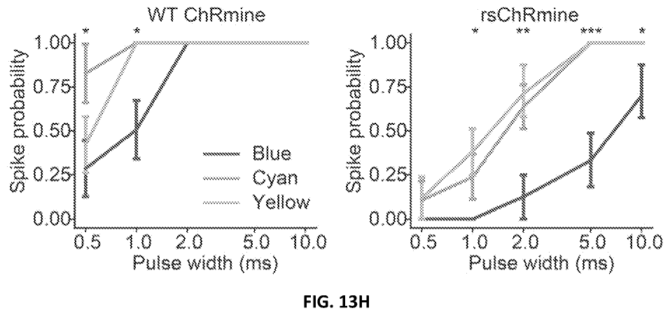

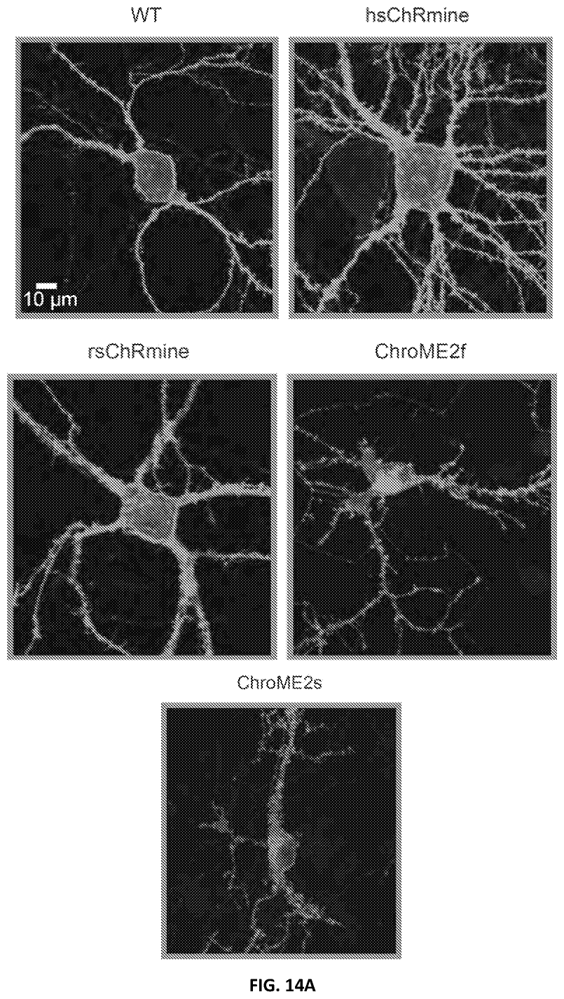

In a separate line of investigation we had found that other mutations predicted to alter pore electrostatic potential can also affect speed (Kato et al., 2018; Kim et al., 2018). However, it was unclear that these mutations along the ion-conduction pathway in dimeric ChRs would translate to the structurally-divergent ChRmine, where ion conduction has distinctive properties [for example, we discovered that ChRmine exhibits high monovalent cation selectivity (excluding Ca²⁺ and Mg²⁺, and strikingly favoring K⁺ over Na⁺; FIG. 13A), an unusual property of this pump-like ChR]. Nevertheless, to test this alternate route, we introduced 6 mutations (FIG. 6A; H33R, D92N, E154Q, E158Q, D242N, E246Q) to the pore, and tested kinetics. Among these, three (H33R, D92N, and E154Q) exhibited powerful effects; most strikingly, τ_{off} and τₒₙ of H33R were more than 2-fold faster (30 ms and 10 ms, respectively) than those of the wild-type (WT) (70 ms and 16 ms, respectively; FIG. 6C), further validating the structure-guided design approach—previously successful for CCRs and ACRs (Deisseroth and Hegemann, 2017)—now for pump-like ChRs.

We next sought to modify the spectral properties, specifically red light actuation to improve compatibility with blue-light-activated genetically encoded Ca²⁺ indicators (GECIs). Previous studies have shown that mutations in the retinal binding pocket (RBP) can change spectral properties, including peak and shape of the action spectrum (Kato et al., 2015a; Oda et al., 2018; Pan et al., 2014); however, sequence identity is low (˜20%) between ChRmine and structurally-resolved CCRs (Marshel et al., 2019), which precluded effective homology modeling of the ChRmine RBP before the structure was solved (FIG. 6B). Since tight packing of RBP residues against the retinal polyene chain and polar interactions with the retinal β-ionone ring have been reported to contribute to red-shifted spectra (Kamiya et al., 2013; Oda et al., 2018; Prigge et al., 2012), we designed 7 constructs with RBP mutations (I146M, G174S, F178Y, I146M/G174S, I146M/F178Y, G174S/F178Y, and I146M/G174S/F178Y) for testing in cultured neurons (FIGS. 13B-13D). Under one-photon (1P) illumination, the double mutant I146M/G174S exhibited a surprisingly large reduction in the blue shoulder and red shift (FIGS. 6D-6G) (with a decrease in cyan light sensitivity of 2.6× and an increase in red light sensitivity of 1.3×, compared to WT ChRmine; FIGS. 13E-6F).

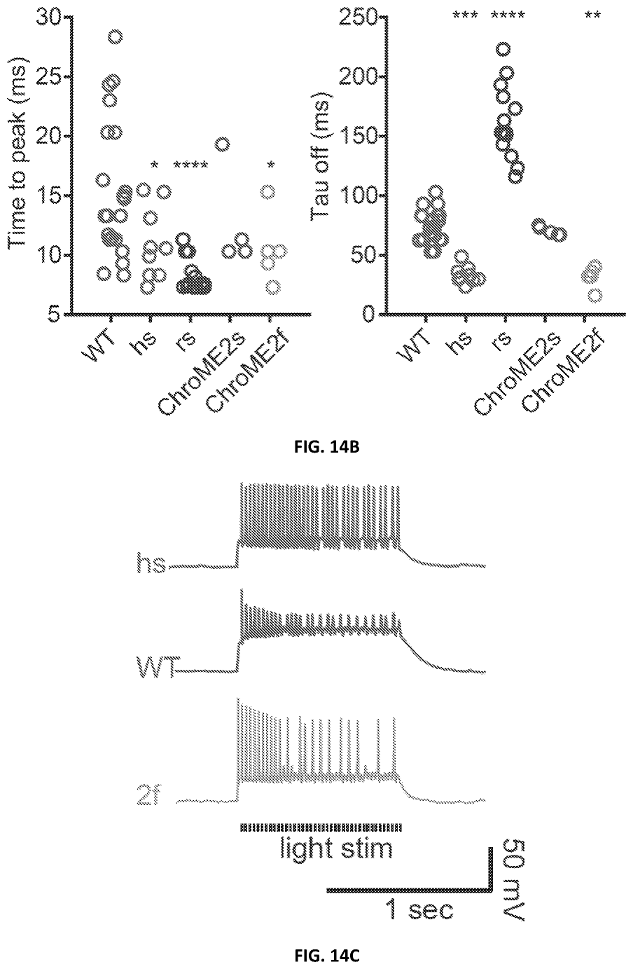

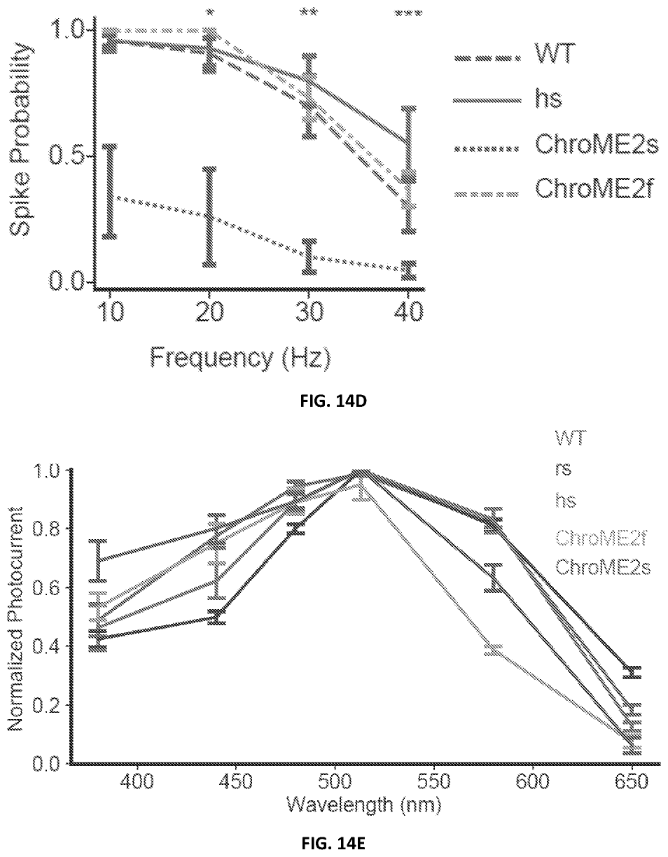

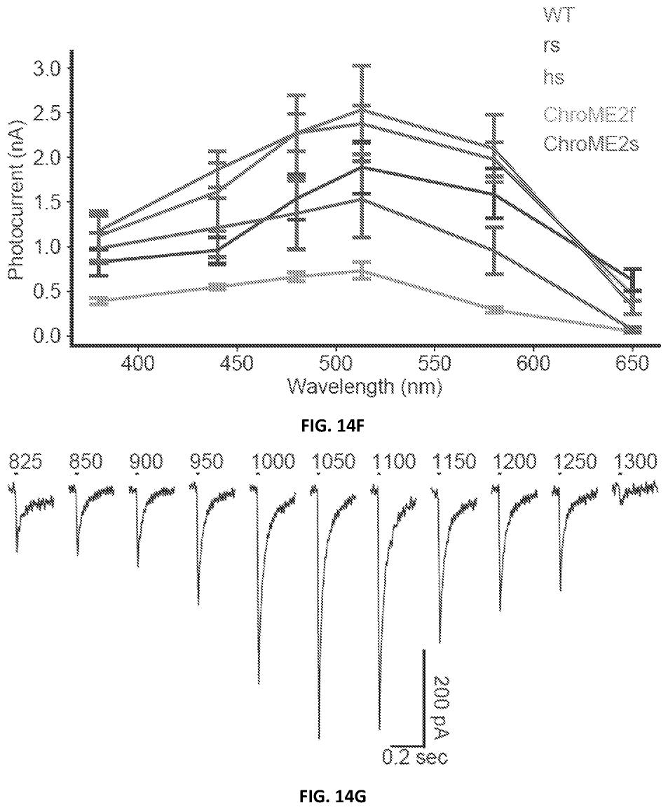

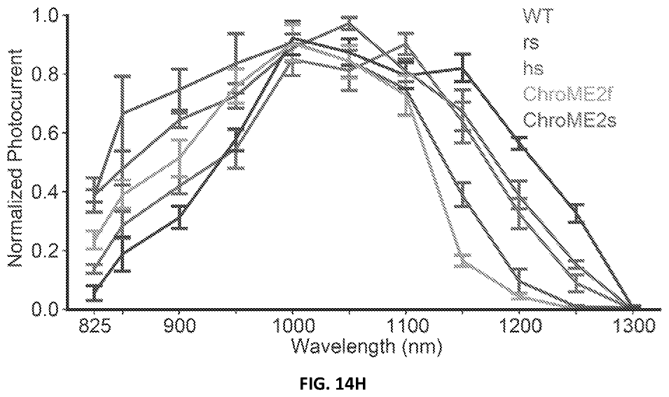

We designated the faster or accelerated-kinetics variant (H33R) as hsChRmine (for high-speed), and the optimal red-shifted variant (I146M/G174S) as rsChRmine (for red-shifted) (FIGS. 6F and 14). Both variants showed robust expression (FIG. 14A), and while many strategies for accelerating channel kinetics and shifting action spectra also reduce photocurrents (Gunaydin et al., 2010; Kato et al., 2018; Mager et al., 2018; Oda et al., 2018), both ChRmine variants exhibited peak photocurrent amplitudes similar to that of WT (FIGS. 6F and 14F). Consistent with its speed (FIG. 14B), we observed that hsChRmine enabled high-fidelity spiking up to 40 Hz (FIGS. 14C-14D; STAR Methods). Combining all three mutations (H33R/I146M/G174S), resulted in further accelerated opening (FIG. 14B) and greater red-shift (FIGS. 14E-14F), albeit with reduced photocurrents (still >500 pA; FIG. 14F); we designate this variant frChRmine (combining fast and further red-shifted performance).

Finally, we compared two-photon (2P) spectra of ChRmine variants with ChroME2 (Sridharan et al., 2021). We found that the spectral shift of rsChRmine was even greater with 2P; at 825 nm, little rsChRmine current was detected, whereas WT ChRmine at 825 nm exhibited 40% of the maximal photocurrent elicited at 1050 nm. Furthermore, the 2P spectra of ChroME2f/2s were found to be blue-shifted relative to ChRmine, as with 1P stim (FIGS. 6G-6H,14E-14H). Together these ChRmine variants provide major practical advantages, compared with opsins that are otherwise good candidates for all-optical interrogation of intact neural circuitry.

Minimal Cross-Talk for all-Optical Experiments with rsChRmine

A blue shoulder persists in the action spectra of all published ChRs; the distinctive spectral properties of rsChRmine thus raised the prospect of minimizing the optical cross-talk that is problematic for all-optical neuroscience. We characterized spike fidelity as a function of pulse width and irradiance for rs- and WT ChRmine in brain slices (FIG. 6I) and cultured neurons (FIGS. 13G-13H); in both settings, rsChRmine exhibited the desired properties. Orange light (580 nm) stimulation of rsChRmine reliably triggered action potentials in brain slices whereas blue light (440 nm) did not (pulse width 1 ms; irradiance 0.3 mW/mm²; FIGS. 6J-6K). In contrast, spike fidelities under orange and blue light for WT ChRmine were similar for all pulse widths and irradiance levels. Thus, the blue shoulder reduction of rsChRmine minimized optical cross-talk, assessed with the crucial readout of neuronal spiking.

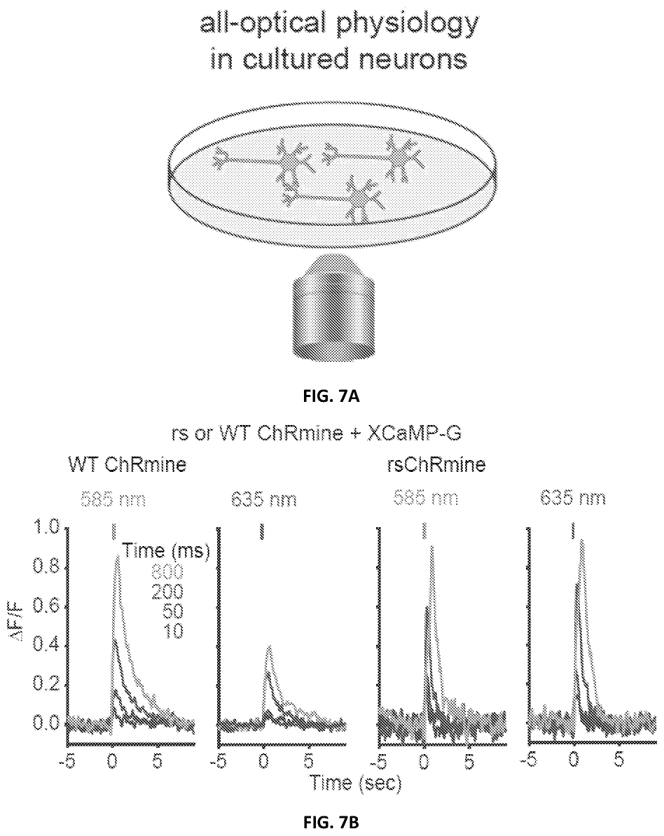

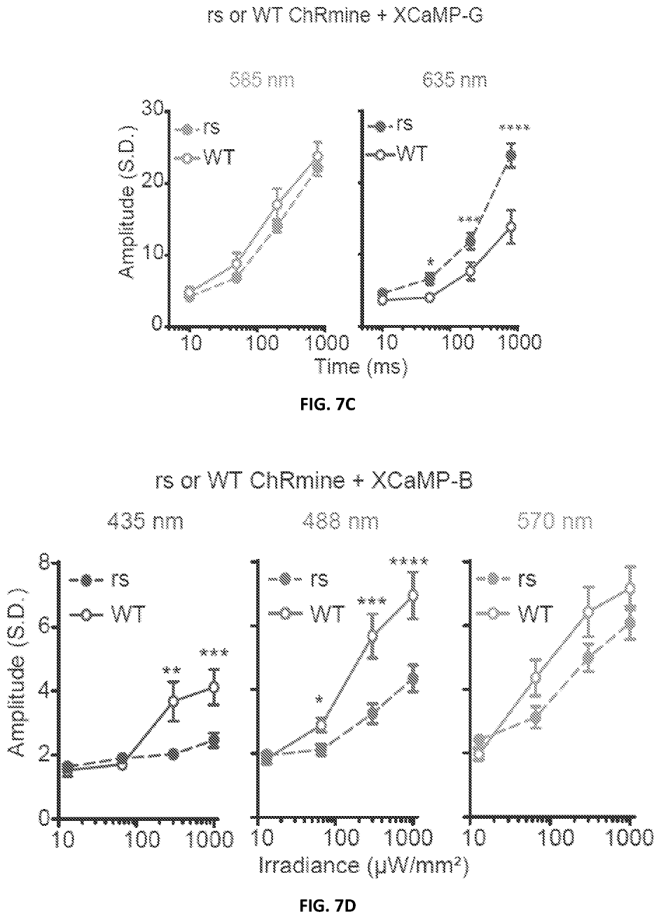

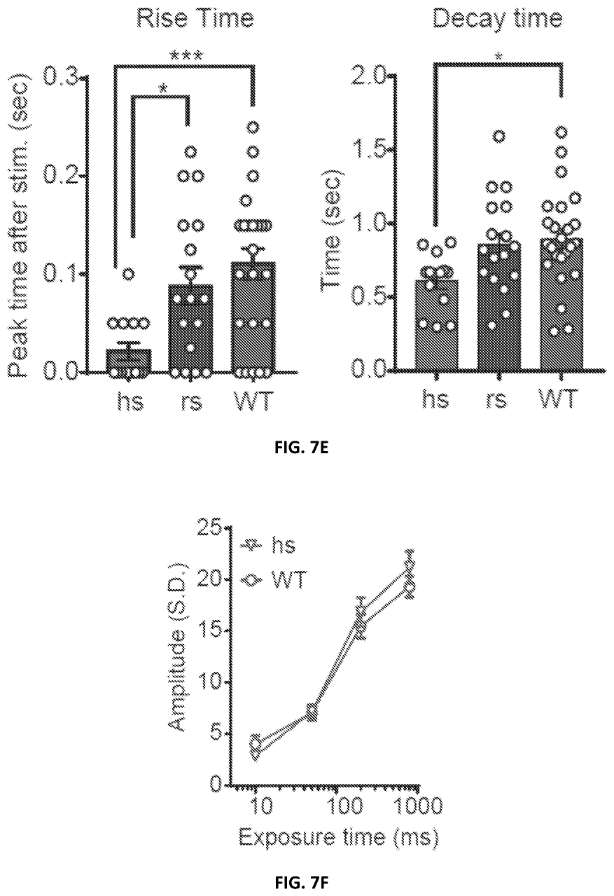

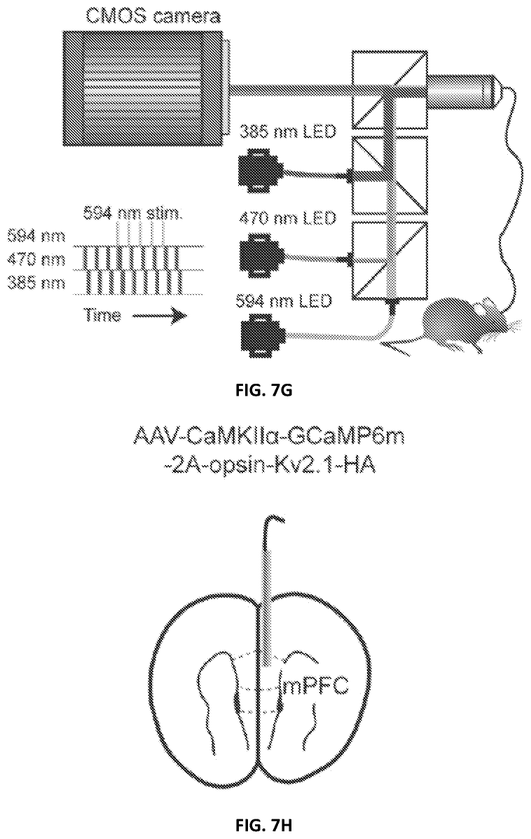

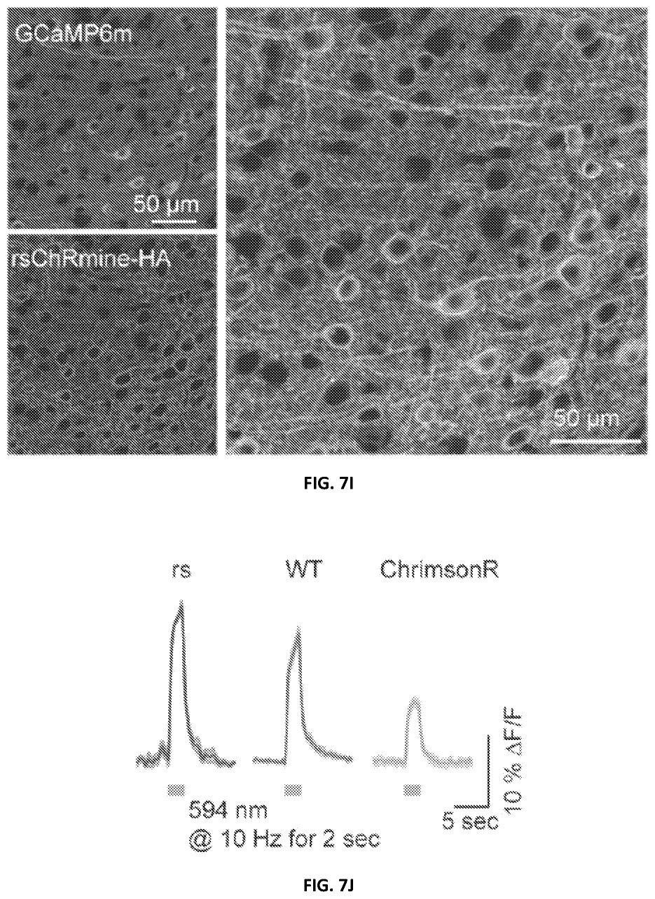

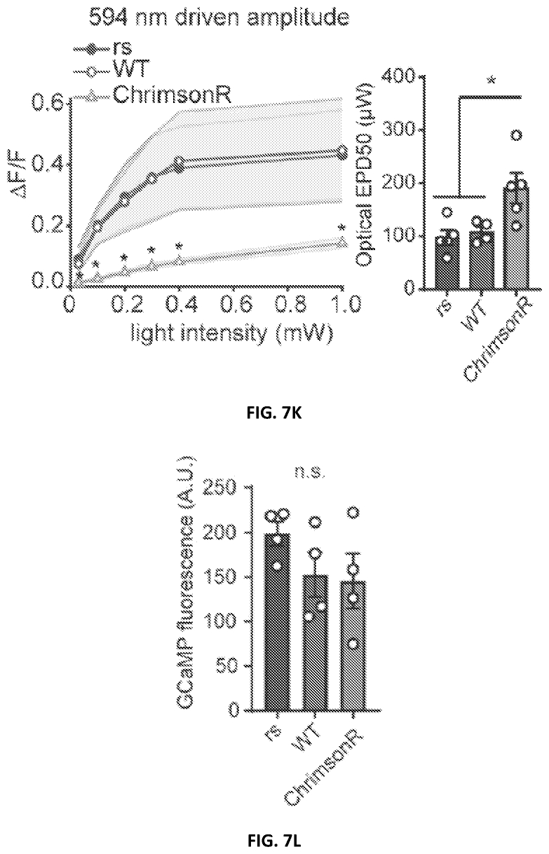

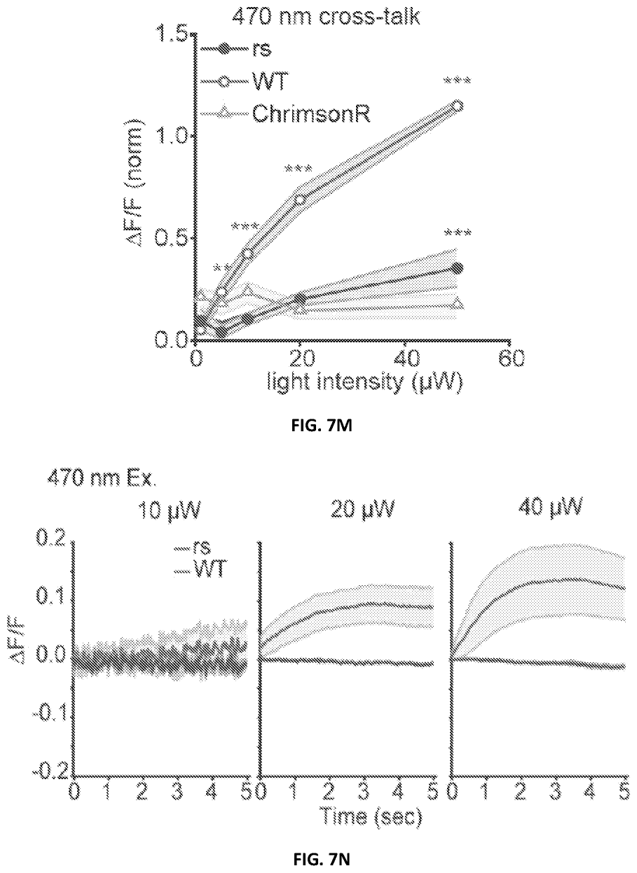

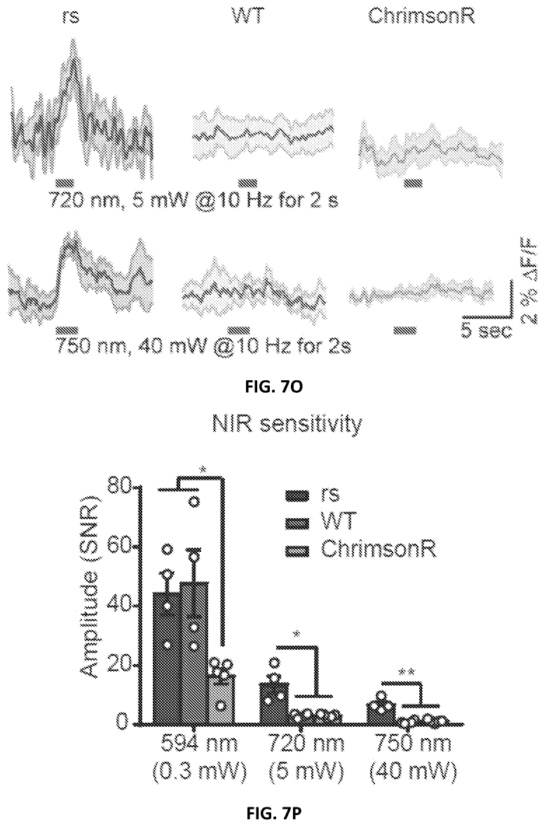

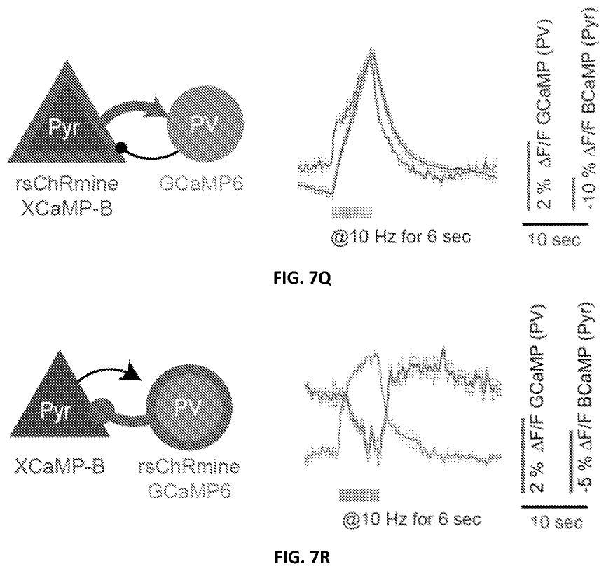

To test potential utility for all-optical experiments, we characterized compatibility with the green GECI XCaMP-G (Inoue et al., 2019) in cultured neurons using 1P stimulation (FIG. 7A). rsChRmine indeed markedly increased responses to red (635 nm) but not orange (585 nm) light (FIGS. 7B-7C). Furthermore, in testing readout from the blue GECI XCaMP-B (Inoue et al., 2019) using an excitation wavelength of 385 nm, we found that rsChRmine exhibited reduced sensitivity to blue (435 nm) and cyan (488 nm) light while no difference was observed for green (570 nm) light (FIG. 7D), revealing that structure-guided design of rsChRmine indeed resulted in suitably optimized properties for all-optical experiments. Also of note, we observed faster kinetics of hsChRmine-elicited XCaMP-G responses compared to those of WT ChRmine, while no difference in response amplitude was measured for the same light-exposure time (FIGS. 7E-7F).

### Example 3—In Vivo Simultaneous Activity Recording and Optogenetic Control in Mice

These results indicated that rsChRmine could be useful for new kinds of simultaneous optical imaging and control in vivo. To explicitly test this, we first applied Frame-Projected Independent-Fiber Photometry (FIP) for simultaneous recording (Kim et al., 2016) and perturbation of activity in pyramidal (Pyr) neurons of medial prefrontal cortex (mPFC) of mice (FIGS. 7G-7H), co-expressing GCaMP6m and opsin to compare rsChRmine with other opsins (WT ChRmine and ChrimsonR; Klapoetke et al., 2014; Marshel et al., 2019; FIG. 7I). We first measured GCaMP6m responses evoked by rsChRmine activation at 594 nm, using interleaved 2.5 μW 470 nm imaging light to measure neural responses. In line with previous findings (Marshel et al., 2019) and our results in cultured neurons (FIGS. 7A-7D), 594 nm light generated markedly larger GCaMP6m response amplitudes (4-fold larger ΔF/F) that were 2× more sensitive to irradiance level in cells expressing rsChRmine or WT ChRmine, compared to those expressing ChrimsonR (FIGS. 7J-7L).

To quantify independence of optical information channels in a practical setting, we tested for incidental stimulation of the targeted cells by 470 nm blue light pulses that are intended for GCaMP imaging, not red-shifted opsin stimulation. rsChRmine- and ChrimsonR-expressing cells exhibited little evoked change in fluorescence even up to 20 μW of 470 nm light, while WT ChRmine exhibited significant fluorescent changes from 3 μW (FIG. 7M). Concordant with this improvement, we also detected a side effect of fluorescence ramping at the beginning of recording with 470 nm imaging light, but only with WT ChRmine (FIG. 7N). rsChRmine was thus distinctive in jointly maximizing redshift and size of photocurrent for a given light level, prompting us to further examine sensitivity and efficacy at even longer wavelengths in vivo. rsChRmine-expressing neurons responded to 720 nm and 750 nm light stimulation, albeit at higher power than with red light, while neurons expressing WT ChRmine and ChrimsonR did not (FIGS. 7O-7P); rsChRmine thus represents the initial ChR reported to drive neural responses in the near-infrared (740 to 1400 nm) illumination band.

Lastly, we asked whether the shifted spectrum of rsChRmine might allow stimulation of activity in a targeted neural population during simultaneous recording of activity in both the stimulated and downstream neural populations. We therefore combined presynaptic rsChRmine stimulation with XCaMP-B recording, alongside postsynaptic GCaMP6f recording, by expressing both rsChRmine and XCaMP-B in Pyr neurons, and GCaMP6f in parvalbumin-expressing (PV) interneurons in mPFC (FIG. 7Q). 1P light was delivered at 380 nm to excite XCaMP-B, 470 nm to excite GCaMP6, and 590 nm to stimulate rsChRmine. After a burst of optical stimulation of Pyr neurons, we were able to track the temporal evolution of excitation in the stimulated Pyr population while also observing downstream activity responses in PV neurons. Conversely, when the targeting strategy was inverted (XCaMP-B to Pyr neurons and GCaMP6m/rsChRmine to PV neurons), the opposite result was obtained: upon optical stimulation of PV neurons, which gave rise to increased activity in the PV-cell population, detected activity of Pyr neurons was potently reduced (FIG. 7R). The use of rsChRmine to measure such a type-to-type impulse response between cell populations in alert animals (and thus obtain a measure of the instantaneous influence of one cell type on another) is a key step toward more controlled and realistic analysis of the complexities of intact-brain dynamics.

### Experimental Methods

### Cloning, Protein Expression, and Purification

Wild-type ChRmine (M1-R304, five amino acids at the C terminus truncated from the previous construct (Marshel et al., 2019)) was modified to include an N-terminal influenza hemagglutinin (HA) signal sequence and FLAG-tag epitope, and C-terminal enhanced green fluorescent protein (eGFP) and 10× histidine tag; the N-terminal and C-terminal tags are removable by human rhinovirus 3C protease cleavage. The construct was expressed in Spodoptera frugiperda (Sf9) insect cells using the pFastBac baculovirus system. Sf9 insect cells were grown in suspension to a density of 3.5×10⁶ cells/mL, infected with ChRmine baculovirus and shaken at 27.5° C. for 24 h. Then, 10 μM all-trans-retinal (ATR) (Sigma-Aldrich) was supplemented to the culture and shaken continued for 24 more hours. The cell pellets were lysed with a hypotonic lysis buffer (20 mM HEPES-NaOH pH 7.5, 20 mM NaCl, 10 mM MgCl₂, 1 mM benzamidine, 1 μg/ml leupeptin, 10 μM ATR), and cell pellets were collected by centrifugation at 10,000×g for 30 min. The above process was repeated twice; then, cell pellets were disrupted by homogenizing with a glass dounce homogenizer in a hypertonic lysis buffer (20 mM HEPES-NaOH pH 7.5, 1 M NaCl, 10 mM MgCl₂, 1 mM benzamidine, 1 μg/ml leupeptin, 10 μM ATR), and crude membrane fraction was collected by ultracentrifugation (45Ti rotor, 125,000×g for 1 h). The above process was repeated twice; then, the membrane fraction was homogenized with a glass douncer in a solubilization buffer (1% n-dodecyl-β-D-maltoside (DDM) (EMD Millipore), 0.2% cholesteryl hemisuccinate (CHS) (Sigma-Aldrich), 20 mM HEPES-NaOH pH 7.5, 500 mM NaCl, 20% glycerol, 5 mM imidazole, 1 mM benzamidine, 1 μg/ml leupeptin) and solubilized for 2 h in 4° C. The insoluble cell debris was removed by centrifugation (125,000×g, 1 h), and the supernatant was mixed with the Ni-NTA superflow resin (QIAGEN) for 1 h in 4° C. The Ni-NTA resin was collected into a glass chromatography column, washed with 2.5 CV wash 1 buffer (0.05% DDM, 0.01% CHS, 20 mM HEPES-NaOH pH7.5, 100 mM NaCl, 50 mM imidazole), 2.5 CV wash 2 buffer (0.05% DDM, 0.06% GDN (glyco-diosgenin), 0.016% CHS, 20 mM HEPES-NaOH pH7.5, 100 mM NaCl, 50 mM imidazole), and 2.5 CV wash 3 buffer (0.06% GDN, 0.006% CHS, 20 mM HEPES-NaOH pH7.5, 100 mM NaCl, 50 mM imidazole), and was eluted in a wash 3 buffer supplemented with 300 mM imidazole. After cleavage of the FLAG tag and eGFP-His₁₀ tag His-tagged 3C protease, the sample was reloaded onto the Ni-NTA column to capture the cleaved eGFP-His₁₀. The flow-through containing ChRmine was collected, concentrated, and purified through gel-filtration chromatography in a final buffer (20 mM HEPES-NaOH pH7.5, 100 mM NaCl, 0.03% GDN, 0.003% CHS).

### Antibody Generation

Mouse monoclonal antibodies against ChRmine were raised according to previously-described methods (Jaenecke et al., 2018). Briefly, a proteoliposome antigen was prepared by reconstituting purified, functional ChRmine at high density into phospholipid vesicles consisting of a 10:1 mixture of chicken egg yolk phosphatidylcholine (egg PC; Avanti Polar Lipids) and the adjuvant lipid A (Sigma-Aldrich) to facilitate immune response. BALB/c mice were immunized with the proteoliposome antigen using three injections at two-week intervals. Antibody-producing hybridoma cell lines were generated using a conventional fusion protocol. Biotinylated proteoliposomes were prepared by reconstituting ChRmine with a mixture of egg PC and 1,2-dipal-mitoyl-sn-glycero-3-phosphoethanolamine-N-(cap biotinyl) (16:0 biotinyl Cap-PE; Avanti), and used as binding targets for conformation-specific antibody selection. The targets were immobilized onto streptavidin-coated microplates (Nunc). Hybridoma clones producing antibodies recognizing conformational epitopes in ChRmine were selected by an enzyme-linked immunosorbent assay on immobilized biotinylated proteoliposomes (liposome ELISA), allowing positive selection of the antibodies that recognized the native conformation of ChRmine. Additional screening for reduced antibody binding to SDS-denatured ChRmine was used for negative selection against linear epitope-recognizing antibodies. Stable complex formation between ChRmine and each antibody clone was checked using fluorescence-detection size-exclusion chromatography. The sequence of the Fab from the antibody clone number YN7002_7 (named as Fab02) was determined via standard 5′-RACE using total RNA isolated from hybridoma cells.

### Formation and Purification of the ChRmine-Fab02 Complex

Purified ChRmine was mixed with a fourfold molar excess of Fab, and the coupling reaction proceeded at 4° C. overnight. The ChRmine-Fab02 complex was purified by size exclusion chromatography on a Superdex 200 increase 10/300 GL column (Cytiva) in 20 mM HEPES-NaOH pH7.5, 100 mM NaCl, 0.03% GDN, 0.003% CHS. Peak fractions were concentrated to about 15 mg/mL for electron microscopy studies.

### Cryo-EM Data Acquisition and Image Processing

Cryo-EM images were acquired at 300 kV on a Krios G3i microscope (Thermo Fisher Scientific) equipped with a Gatan BioQuantum energy filter and a K3 direct detection camera in the electron counting mode. The movie dataset was collected in a correlated double sampling (CDS) mode, using a nine-hole image shift strategy in the SerialEM software (Mastronarde, 2005b), with a nominal defocus range of 0.8 to 1.6 μm. The 3,528 movies were acquired at a dose rate of 6.3 e⁻/pixel/s, at a pixel size of 0.83 Å and a total dose of 46 e⁻/Å².

Image processing was performed in RELION-3.1 (Zivanov et al., 2018). Beam-induced motion correction and dose weighting were performed with RELION's implementation of the MotionCor2 algorithm (Zheng et al., 2017), and CTF parameters were estimated with CTFFIND-4.1.13 (Rohou and Grigorieff, 2015). Particles were first picked using the Laplacian-of-gaussian algorithm, and 2D class average images were generated as templates for reference-based auto-picking. Reference-based picked 2,958,159 particles were subjected to several rounds of 2D and 3D classifications. The selected 555,801 particles were subjected to a 3D auto-refinement, resulting in a 2.8 Å map. Subsequently, Bayesian polishing (Zivanov et al., 2019) and CTF refinement (Zivanov et al., 2020), followed by a 3D auto-refinement, resulted in a 2.6 Å map. Micelle and constant regions of Fab fragments densities were subtracted from particle images, and the subtracted particles were subjected to a masked 3D classification without alignment. After a 3D auto-refinement of selected 185,895 particles, three runs of CTF refinement were performed in order as follows: refining magnification anisotropy; refining optical aberrations; refining per-particle defocus and per-micrograph astigmatism. Another round of 3D auto-refinement yielded a 2.13 Å map. These particles were subjected to, a second round of Bayesian polishing, CTF refinement and a transmembrane region-focused 3D auto-refinement with the reconstruction algorithm SIDESPLITTER (Ramlaul et al., 2020), resulting in the final map at a global resolution of 2.02 Å.

### Model Building and Refinement

An initial model was formed by rigid body fitting of the C1C2 (PDB: 3UG9) (Kato et al., 2012). This starting model was then subjected to iterative rounds of manual and automated refinement in Coot (Emsley and Cowtan, 2004) and Refmac5 (Murshudov et al., 2011) in Servalcat pipeline (Yamashita et al., 2021), respectively. The Refmac5 refinement was performed with the constraint of C3 symmetry. The final model was visually inspected for general fit to the map, and geometry was further evaluated using Molprobity (Chen et al., 2010). The final refinement statistics is summarized in Table 1. All molecular graphics figures were prepared with UCSF Chimera (Pettersen et al., 2004), UCSF ChimeraX (Goddard et al., 2018), and Cuemol (see world-wide-website: cuelmol.org).

### Analysis of the Structures

The ion-conducting pores were calculated by the software HOLLOW using a grid-spacing of 1.0 Å. The electrostatic potentials of the pores are calculated by PDB2PQR server (Baker et al., 2001; Dolinsky et al., 2004). Trimer opening radii of the ChRmine was calculated with HOLE.

### High Performance Liquid Chromatography (HPLC) Analysis of Retinal Isomers

The retinal isomers were analyzed with an HPLC system equipped with a silica column (particle size 3 μm, 150×6.0 mm; Pack SIL, YMC, Japan), a pump (PU-4580, JASCO, Japan) and a UV-Visible detector (UV-4570, JASCO, Japan). The purified sample in a buffer containing 20 mM HEPES-NaOH pH 7.5, 100 mM NaCl, 0.035% GDN, 0.0035% CHS (GDN:CHS=10:1) were dark-adapted for two days at 4° C. A 75 μL sample and 280 μL of 90% (v/v) methanol aqueous solution were mixed on ice and then 25 μL of 2 M hydroxylamine (NH₂OH) was added to convert retinal chromophore into retinal oxime, which was extracted with 800 μL of n-hexane. A 200 μL of the extract was injected into the HPLC system. The solvent containing 15% ethyl acetate and 0.15% ethanol in hexane was used as a mobile phase at a flow rate of 1.0 mL min⁻¹. Illumination was performed on ice with green light (530±5 nm) for 20 s for samples under illumination and 60 s for light adaptation. The molar composition of the sample was calculated from the areas of the peaks and the molar extinction coefficients at 360 nm (all-trans-15-syn: 54,900 M⁻¹ cm⁻¹; all-trans-15-anti: 51,600 M⁻¹ cm⁻¹; 13-cis-15-syn, 49,000 M⁻¹ cm⁻¹; 13-cis-15-anti: 52,100 M⁻¹ cm⁻¹; 11-cis-15-syn: 35,000 M⁻¹ cm⁻¹; 11-cis-15-anti: 29,600 M⁻¹ cm⁻¹) (Trehan et al., 1990).

### Preparation of Lipid-Reconstituted ChRmine for High-Speed AFM Imaging

For HS-AFM imaging of lipid-reconstituted ChRmine, we applied membrane scaffolding proteins (MSP), which were developed for nanodisc technology (Bayburt et al., 2002; Denisov and Sligar, 2016). We followed the manufacturer's protocol for the nanodisc (Sigma-Aldrich, St. Louis, MO, USA) with minor modifications as described previously (Shibata et al., 2018). Briefly, for reconstituted lipids, we used a mixture of phospholipids, asolectin from soybean (Sigma-Aldrich, No. 11145). Asolectin (120 μg) was dissolved in chloroform and then evaporated under N2 gas to completely remove the solvent. Then, the lipids were suspended in 50 μL buffer A (20 mM HEPES-KOH pH 7.4, 100 mM NaCl, and 4% DDM) and sonicated for ˜1 min with a tip-sonicator. Next, dissolved membrane proteins (1 nmol) and MSP (50 μL, 1 mg/mL) (MSP1E3D1, Sigma-Aldrich, No. M7074) were added to the lipid suspension and mixed for ˜1 h while rotating in the dark at 4° C. Finally, we added 60 mg Bio-beads SM-2 (Bio-Rad, Hercules, CA, USA, No. 1523920) and dialyzed the samples in detergent overnight at 4° C. According to the manufacturer's protocol, nanodisc samples should be fractionated on a column to purify the nanodiscs based on size (˜10 nm in diameter). Here, we did not purify the reconstituted samples, but obtained flat membranes with limited sizes<30 nm in diameter.

### High-Speed AFM Measurements

A homemade HS-AFM operated in tapping mode was used (Shibata et al., 2017, 2018). An optical beam deflection detector detected the cantilever (Olympus, Tokyo, Japan: BL-AC10DS-A2) deflection using an infrared (IR) laser at 780 nm and 0.7 mW. The IR beam was focused onto the back side of the cantilever covered with a gold film through a ×60 objective lens (Nikon, Tokyo, Japan: CFI S Plan Fluor ELWD 60×). The reflected IR beam was detected by a two-segmented PIN photodiode. The free oscillation amplitude of the cantilever was ˜1 nm and set-point amplitude was approximately 90% of the free amplitude for feedback control of HS-AFM observation. An amorphous carbon tip (˜500 nm length), grown by electron beam deposition by scanning electron microscope, was used as an AFM probe. As a HS-AFM substrate, a mica surface treated with 0.01% (3-aminopropyl) triethoxysilane (Shin-Etsu Silicone, Tokyo, Japan) was used. All HS-AFM experiments were carried out in buffer solution containing 20 mM Tris-HCl pH 8.0 and 100 mM NaCl at room temperature (24-26° C.) and data analyses were conducted using laboratory-developed software based on IgorPro 8 software (WaveMetrics, USA). We usually used a scan area of 43×32 nm² with 130×95 pixels. HS-AFM images were captured at frame rates of 2 fps. All HS-AFM images were processed by Gaussian noise-reduction filters.

### Measurement of UV Absorption Spectra

For pH titration, the final purified product (20 mM HEPES-NaOH pH7.5, 100 mM NaCl, 0.03% GDN, 0.003% CHS) was diluted with 100 mM of the respective pH buffer (StockOptions pH Buffer Kit), and the UV-Vis spectra were measured.

### Laser Flash Photolysis

For the laser flash photolysis spectroscopy, wildtype ChRmine was solubilized in 20 mM HEPES-NaOH pH 7.5, 100 mM NaCl, 0.035% GDN, 0.0035% CHS (GDN:CHS=10:1) or 20 mM sodium acetate pH 4.0, 100 mM NaCl, 0.03% GDN, 0.003% CHS (GDN:CHS=10:1), and ChRmine D115N and D253N mutants were solubilized in 20 mM sodium acetate pH 4.0, 100 mM NaCl, 0.03% GDN, 0.003% CHS (GDN:CHS=10:1). The optical density of the protein solution was adjusted to ˜0.4 (protein concentration ˜0.28 mg/mL) at the absorption maximum wavelengths. The laser flash photolysis measurements were conducted as previously described (Inoue et al., 2013). ChRmine wildtype at pH 7.5 was excited by the second harmonics of a nanosecond-pulsed Nd³⁺-YAG laser (excitation wavelength (λ_{exc})=532 nm, 4.5 mJ/pulse, 1.4-0.5 Hz, INDI40, Spectra-Physics, CA), and nano-second pulse from an optical parametric oscillator (4.5 mJ/pulse, basiScan, Spectra-Physics, CA) pumped by the third harmonics of Nd³⁺-YAG laser (λ=355 nm, INDI40, Spectra-Physics, CA) was used for the excitation of ChRmine wildtype at pH 4.0 (λ_{exc}=505 nm), ChRmine D115N (λ_{exc}=488 nm) and D253N (λ_{exc}=500 nm). Transient absorption spectra were obtained by monitoring the intensity change of white-light from a Xe-arc lamp (L9289-01, Hamamatsu Photonics, Japan) passed through the sample with an ICCD linear array detector (C8808-01, Hamamatsu, Japan). To increase the signal-to-noise (S/N) ratio, 45-60 spectra were averaged, and the singular-value-decomposition (SVD) analysis was applied. To measure the time-evolution of transient absorption change at specific wavelengths, the light of Xe-arc lamp (L9289-01, Hamamatsu Photonics, Japan) was monochromated by monochromators (S-10, SOMA OPTICS, Japan) and the change in the intensity after the photo-excitation was monitored with a photomultiplier tube (R10699, Hamamatsu Photonics, Japan). To increase S/N ratio, 100-200 signals were averaged. The time-evolution of transient absorption change was analyzed by global multi-exponential fitting to determine the time constant of each reaction step and absorption spectra of the photo-intermediates. Some reaction steps were reproduced by double or triple exponentials. In this case, the averaged time constant calculated by

$${τ}_{a v e} = \frac{{\sum }_{i} {A}_{i} / {k}_{i}}{{\sum }_{i} {A}_{i}}$$

where Aᵢ and kᵢ are the amplitude at a wavelength of representing the M-intermediate and the rate constant of i-th exponential function (i=1-3).

### Molecular Cloning

All ChRmine mutant plasmids were constructed in AAV-CaMKIIa or pcDNA 3.1 backbones using overlapping PCR as described previously (Fenno et al., 2020; Marshel et al., 2019). ChRmine-Oscarlet-Kv2.1, WT, rs and hs mutants were transferred to an Elav3 backbone using AgeI and MluI sites for creating transgenic fish lines. Every plasmid was sequence-verified.

### Primary Cell Transfection

For neuronal transfection, 2.0 μg plasmid DNA was mixed with 1.875 μL 2 M CaCl₂) (final Ca²⁺ concentration 250 mM) in 15 μL H₂O. To DNA-CaCl₂ we added 15 μL of 2×HEPES-buffered saline pH 7.05. After 20 min at room temperature (20-22° C.), the mix was added dropwise into each well (from which the growth medium had been removed and replaced with pre-warmed minimal essential medium (MEM)) and transfection proceeded for 45-60 min at 37° C., after which each well was washed with 3×1 ml warm MEM before the original growth medium was returned. Neurons were allowed to express transfected DNA for 6-8 days prior to experiments.

For HEK cell transfection, 0.8 μg plasmid DNA was mixed with 2 μL Lipofectamine 2000 (Invitrogen) in 100 μL Opti-MEM (Invitrogen, incubated at room temperature (20-22° C.) for 20 minutes, and the mix was added dropwise into each well (from which the growth medium had been removed and replaced with 400 μL pre-warmed Opti-MEM). Transfection proceeded for two hours at 37° C., after which the transfection media was replaced by normal HEK cells growth media. Cells were allowed to express transfected DNA for 2-3 days prior to experiments.

### Virus Production

AAV-8 (Y733F), was produced by the Stanford Neuroscience Gene Vector and Virus Core. In brief, AAV8 was produced by standard triple transfection of AAV 293 cells (Agilent). At 72 h post transfection, the cells were collected and lysed by a freeze-thaw procedure. Viral particles were then purified by an iodixanol step-gradient ultracentrifugation method. The iodixanol was diluted and the AAV was concentrated using a 100-kDa molecular mass-cutoff ultrafiltration device. Genomic titer was determined by quantitative PCR. All viruses were tested in cultured neurons for expected expression patterns prior to use in vivo.

### In Vitro Electrophysiology in HEK293 Cells

HEK293 cells transfected with pcDNA3.1(+) plasmids were placed in an extracellular tyrode medium (150 mM NaCl, 4 mM KCl, 2 mM CaCl₂), 2 mM MgCl₂, 10 mM HEPES pH 7.4, and 10 mM glucose). Borosilicate patch pipettes (Harvard Apparatus) with resistance of 4-6 Mohm were filled with intracellular medium (140 mM potassium-gluconate, 10 mM EGTA, 2 mM MgCl₂ and 10 mM HEPES pH 7.2). Light was delivered with the Spectra X Light engine (Lumencor) connected to the fluorescence port of a Leica DM LFSA microscope with a 580 nm filter for orange light generation.

Channel kinetics and photocurrent amplitudes were measured in voltage clamp mode at −70 mV holding potential. To determine channel kinetics and photocurrent amplitudes, traces were first smoothed using a lowpass Gaussian filter with a −3 dB cutoff for signal attenuation and noise reduction at 1,000 Hz and then analyzed in Clampfit software (Axon Instruments). Liquid junction potentials were corrected using the Clampex built-in liquid junction potential calculator as previously described. Statistical analysis was performed with t-test or one-way ANOVA, and the Kruskal-Wallis test for non-parametric data, using Prism 7 (GraphPad) software. Data collection across opsins was randomized and distributed to minimize across-group differences in expression time, room temperature, and related experimental factors.

### Ion Selectivity Testing in HEK293 Cells

HEK293 cells and devices for the measurement were prepared as described in the previous section. For the high sodium extracellular/high potassium intracellular condition, we used sodium bath solution containing 120 mM NaCl, 4 mM KCl, 2 mM CaCl₂), 2 mM MgCl₂, and 10 mM HEPES pH 7.2 (with glucose added up to osm 310 mOsm), along with potassium pipette solution containing 120 mM KCl, 10 mM EGTA, 4 mM NaCl, 2 mM CaCl₂), 2 mM MgCl₂, and 10 mM HEPES pH 7.2 (with glucose added up to osm ˜290). For the high potassium extracellular/high sodium intracellular condition, NaCl and KCl concentrations were reversed, and all other ionic concentrations were kept constant. For ion selectivity measurements, ions in both bath and pipette solutions were replaced with either 120 mM NaCl, 120 mM KCl, 80 mM CaCl₂), 80 mM MgCl₂ or 120 mM NMDG-Cl, with all other components at low concentrations (4 mM NaCl, 4 mM KCl, 2 mM CaCl₂), 2 mM MgCl₂, and 10 mM HEPES). Glucose was added to increase intracellular solution to 310 mOsm and extracellular solution to 290 osm. Photocurrent amplitudes were measured at −70 mV holding membrane potential. Equilibrium potentials were measured by holding membrane potentials from −75 mV to +45 mV in steps of 10 mV.

### In Vitro One-Photon Electrophysiology in Cultured Hippocampal Neurons

Primary rat hippocampal cultured neurons were transfected with pAAV ChRmine-bearing plasmids and were measured in the same setup as described in the HEK293 electrophysiology section. Voltage clamp recordings were performed in the presence of bath-applied tetrodotoxin (TTX, 1 μM, Tocris). For screening of action spectra, cells were held at resting potential of −70 mV, with 1.0 mW/mm² light delivery for 1 sec at wavelengths (in nm) of 390, 438, 485, 513, 585 and 650, which were generated using filters of corresponding peak wavelengths and 15-30 nm bandwidth. Channel kinetics and photocurrent amplitudes were measured at −70 mV holding membrane potential. Liquid junction potentials were corrected using the Clampex built-in liquid junction potential calculator as previously described. Current clamp measurements were performed in the presence of glutamatergic synaptic blockers: 6-cyano-7-nitroquinoxaline-2,3,-dione (CNQX; 10 μM, Tocris) and D(−)-2-amino-5-phosphonovaleric acid (APV; 25 μM, Tocris).

For light pulse-width experiments, 585 nm light with 5 Hz frequency and 0.7 mW/mm² intensity was used at varying pulse-width values (in ms) of 0.5, 1, 2, 5 and 10. For light sensitivity experiments, 585 nm light with 5 Hz frequency and 5 ms pulse-width was used at varying light power densities (in mW/mm²) of 0.003, 0.01, 0.03, 0.1, 0.3, 0.7, and 1.0. For spike fidelity experiments, 585 nm light with 0.7 mW/mm² power density was used, with 1 ms pulse-width for ChRmine variants. Data collection across opsins was randomized and distributed to minimize across-group differences in expression time, room temperature, and related experimental factors. Statistical analysis was performed with t-test or one-way ANOVA, and the Kruskal-Wallis test with Dunn's test for multiple comparisons for non-parametric data, using Python and Prism 7 (GraphPad) software.

### In Vitro Two-Photon Electrophysiology

All two-photon electrophysiology experiments were performed with cultured hippocampal neurons in the same intracellular and extracellular solutions as for one-photon electrophysiology characterization. Experiments were conducted on a commercial microscope (Bruker Ultima running PrairieView v5.4) using a Nikon 16×/0.8 NA (CFI75) long-working distance objective for light delivery. For two-photon stimulation, spiral scanning was performed through a defined spiral ROI with 15 μm diameter, with 10 rotations per spiral, and 1.3 ms total exposure duration with 80 MHz laser repetition rate (Coherent Discovery). The axial point-spread-function FWHM of the two-photon stimulation beam was measured to be 6.9+/−0.2 μm at 920 nm using 1 μm diameter beads (Invitrogen Focal Check Slide #1, F36909).

For two-photon action spectra characterization, recordings were conducted in voltage clamp mode at holding voltage of −75 mV. Action spectra were measured in randomized trial order at wavelengths (in nm) of 825, 850, 900, 950, 1000, 1050, 1100, 1150, 1200, 1250, and 1300 at a laser power of 20 mW. 10 rotations/spiral, 15 mm diameter spirals, 1.3 ms duration, and 80-MHz laser repetition rate. We measured the focal shift as we systematically varied wavelengths from 825 to 1300 nm and found that there was a ˜25 um difference in focus between 825 and 1300 nm. Therefore, the z-focus was adjusted to compensate for empirically measured focal shifts during randomized wavelength delivery.

No steps were taken to compensate for the potential effects of pulse broadening due to spectral dispersion. All measurements were normalized by the maximum value of the single recording session and then averaged across cells.

### Stereotactic Surgeries

A midline incision was made to expose the skull and small craniotomies were made above the injection sites using a Meisinger Carbide Burr size 1/4. All virus dilutions were performed in ice-cold PBS and all viruses were produced at the Stanford Gene and Viral Vector Core. Virus injections were delivered with a 10 μL syringe (World Precision Instruments) and 33-gauge beveled needle (World Precision Instruments), injected at 100 nL min⁻¹ using an injection pump (World Precision Instruments). For slice physiology experiments, mice were injected with either AAV8-CaMKIIα-ChRmine-p2A-oscarlet (2.0e13 vg/mL) or AAV8-CaMKIIα-rsChRmine-p2A-Oscarlet (7.30e12 vg/mL). One microliter of virus was stereotactically injected bilaterally into the motor cortex of 8-12 week old mice at 1.7 mm AP, 0.75 mm ML, and 1.5 mm DV from the bregma. For fiber photometry experiments, mice were injected with either AAV8-CaMKIIα-GcaMP6m-2A-opsin where “opsin” is one of the three opsins shown in FIGS. 7G-7R: rsChRmine (1.0e12 vg/ml), WT ChRmine (1.0e12 vg/ml), or ChrimsonR (1.0e12 vg/ml). For type-to-type experiments, PV-2a-Cre mice were injected with either a mixture of AAV8-CaMKIIα-rsChRmine-oScarlett-Kv2.1 (3.0e12 vg/ml), AAV8-CaMKIIα-XcaMP-B (8.0e12 vg/ml), and AAVdj-EF1α-DIO-GcaMP6f (3.0e12 vg/ml) or a mixture of AAV8-CaMKIIα-XcaMP-B (8.0e12 vg/ml) and AAV8-EF1α-DIO-GcaMP6m-2A-rsChRmine (5.0e11 vg/ml). 0.7 μl of virus was stereotactically injected unilaterally into the mPFC of 8-12 week old mice at 1.8 mm AP, 0.35 mm ML, and 2.4 mm DV from the bregma. Following injection, the injection needle was held at the injection site for 10 min then slowly withdrawn. Mice were administered 0.5-1.0 mg kg⁻¹ subcutaneous buprenorphine-SR (ZooPharma) approximately 30 min before the end of the surgery for post-operative pain management.

### Acute Slice Electrophysiology

Recordings of rsChRmine and ChRmine-expressing pyramidal cells were performed in acute slices from wild-type C57BL/6 mice 4-5 weeks after virus injection. Coronal slices 300 μm in thickness were prepared after intracardial perfusion with ice-cold N-methyl-d-glutamine (NMDG) containing cutting solution: 93 mM NMDG, 2.5 mM KCl, 25 mM glucose, 1.2 mM NaH₂PO₄, 10 mM MgSO₄, 0.5 mM CaCl₂), 30 mM NaHCO₃, 5 mM Na ascorbate, 3 mM Na pyruvate, 2 mM thiourea and 20 mM HEPES pH 7.3-7.4. Slices were incubated for 12 min at 34° C., and then were transported to room temperature oxygenated artificial cerebrospinal fluid (ACSF) solution: 124 mM NaCl, 2.5 mM KCl, 24 mM NaHCO₃, 2 mM CaCl₂), 2 mM MgSO₄, 1.2 mM NaH₂PO₄, 12.5 mM glucose and 5 mM HEPES pH 7.3-7.4.

Current clamp measurements were performed as described in the in vitro electrophysiology section. Briefly, 585 nm light with 5 Hz frequency and 0.7 mW/mm² intensity was used at varying pulse-width values (in ms) of 0.5, 1, 2, 5 and 10 to test pulse width, and 585 nm light with 5 Hz frequency and 5 ms pulse-width was used at varying light power densities (in mW/mm²) of 0.003, 0.01, 0.03, 0.1, 0.3, 0.7, and 1.0. For spike fidelity experiments, 585 nm light with 0.7 mW/mm² power density was used, with 1 ms pulse-width for ChRmine variants. Data collection across opsins was randomized and distributed to minimize across-group differences in expression time, room temperature, and related experimental factors.

In Vitro Characterization Preparatory to all-Optical Set-Up

Dissociated hippocampal neurons were cultured and infected with both red-shifted opsin variants and XcaMP-G or XcaMP-B as previously described (Marshel et al., 2019). One microliter viral suspension of WT ChRmine (AAV8-CaMKIIα-ChRmine-oScarlet-Kv2.1, 1.3e13 vg/mL), rsChRmine (AAV8-CaMKIIα-rsChRmine-oScarlet-Kv2.1, 8.8e12 vg/mL), or hsChRmine (AAV8-CaMKIIα-hsChRmine-oScarlet-Kv2.1, 1.8e13 vg/ml) mixed with 1 μL XcaMP-G (AAV8-CaMKIIα-XcaMP-G, 6.9e12 vg/mL)) or 1 μL XcaMP-B (AAV8-CaMKIIα-XcaMP-B, 2.4e13 vg/mL) was added after 5 DIV. Cultured neurons were used between 12 and 14 DIV for experiments. Coverslips of cultured neurons were transferred from the culture medium to a recording bath filled with Tyrode's solution containing (129 mM NaCl, 5 mM KCl, 30 mM glucose, 25 mM HEPES-NaOH pH 7.4, 1 mM MgCl₂ and 3 mM CaCl₂)) supplemented with 10 μM CNQX and 25 μM APV to prevent contamination from spontaneous and recurrent synaptic activity. Optical stimulation and imaging were performed using a 40×/0.6-NA objective (Leica), sCMOS camera (Hamamatsu, ORCA-Flash4.0) and LED light source (Spectra X Light engine, Lumencor), all coupled to a Leica DMI 6000 B microscope. XcaMP-B or XcaMP-G were excited by 390 nm (Semrock, FF01-390/18) or 488 nm (Semrock, LL01-488-12.5), respectively, with the Spectra X Light engine. XcaMP-B emission was reflected off a quad wavelength dichroic mirror (Semrock, FF409/493/573/652-Di02) for various color light stimulation, and passed through a triple-band emission filter (Semrock, FF01-432/523/702-25). XcaMP-G emission was reflected off a dual wavelength dichroic mirror (Chroma, ZT488/594rpc) for orange light stimulation or another mirror (ZT488/640rpc) for red light stimulation, and passed through a 535-30-nm emission filter (Chroma, ET535/30m). Red-responsive opsins were activated with a Spectra X Light engine filtered either with 585 nm orange light (Semrock, FF01-585/29-25, 2.0 mW/mm²) or 635 nm red light (Semrock, FF01-635/18-25, 2.0 mW/mm²). For light sensitivity experiments, 434 nm blue light (Semrock, 434/17), 488 nm cyan light (Semrock, LL01-488-12.5), or 570 nm green light (Chroma, HQ570/20m) with 400 ms pulse-width was used at varying light power densities (in mW/mm²) of 0.013, 0.066, 0.30 and 1.0.

Fluorescence of XcaMP-B or XcaMP-G was imaged using low-intensity 385 nm (10 μW/mm²) or 488 nm (8 μW/mm²) laser light, respectively, without substantially activating red-responsive opsin. Images were acquired at 20 Hz using MicroManager (http://micro-manager.org). Light for stimulation was controlled by LabVIEW (National Instruments) and applied every 10 sec at an exposure time of 10, 50, 200 and 800 msec. Imaging data were analyzed in MATLAB (MathWorks). Circular regions of interest (ROIs) were drawn manually based on the averaged image. We performed background subtraction before calculating Ca²⁺ signals. ΔF/F responses were calculated to normalize the signal in each ROI, by dividing by its mean value of total fluorescence intensity and subtracting 1. Noise was calculated as the standard deviation of the total ΔF/F fluctuation 3 sec before the stimulation. Signal-to-noise ratio (SNR) was then computed as ΔF/F response divided by noise. Peak amplitude was calculated from the maximum value during 2 sec after stimulus cessation. To compare red-responsive opsins to triggered XcaMP-G kinetics, we calculated 200 msec exposure-triggered Ca²⁺ transients. Rise time (tpeak) was defined as the time-to-peak from the cessation of the light stimulus to the time point at which maximal-amplitude fluorescence was reached. The decay constants (tau) were determined by single-exponential fit from the peak of the fluorescence response for 2 sec after stimulation.

### FIP Setup and Analysis

We collected bulk fluorescence from targeted brain regions using a single optical fiber while delivering excitation light for fiber photometry as described previously (Inoue et al., 2019; Kim et al., 2016). We have extended these methods to the case of dual excitation wavelengths (380 and 470 nm) with stimulation wavelength (590, 720, or 750 nm) delivered through the same fiber to allow tracking of activity in distinct cell populations (sender and receiver) during optogenetic stimulation of the sender population. A low fluorescence 400-mm-diameter 0.66-NA mono fiberoptic cannula (Doric Lenses) was implanted above mPFC for fiber photometry. Cannulas were secured to the skull using a base layer of adhesive dental cement (C&B-Metabond, Parkell), followed by a second layer of cranioplastic cement (Ortho-Jet, Lang). Experiments were conducted 4-6 weeks later for FIP recordings to allow for sufficient viral expression and postsurgery recovery. One end of the patchcord terminated in an SMA connector (Thorlabs, SMISMA) mounted at the working distance of the objective, and the other end terminated in 2.5-mm-diameter stainless steel ferrules. These ferrules were coupled via bronze sleeves (Doric, SLEEVE_BR_2.5) to ferrules implanted into a mouse. Fiber faces were imaged through a 20×/0.75-NA objective (Nikon, CFI Plan Apo Lambda 20×) through a series of reconfigurable dichroic mirrors.

In the standard configuration, the three LEDs (M385F1, M470F3, and M595F2, Thorlabs) were filtered with 380-14 nm, 473 nm, and 586-20 nm bandpass filters (FF01-380/14-25, LL01-473-25, and FF01-586/20-25, Semrock). Excitation and optogenetic stimulation light from two sources (470 and 590 nm) was passed to a 525 nm longpass dichroic mirror (T525lpxr, Chroma), and then combined with 380 nm light using a second 425 nm longpass dichroic (T425lpxr, Chroma) before finally being coupled into the optical fiber patch cord using a triple multiband dichroic (69013bs, Chroma). Fluorescence emission passed through multi-bandpass fluorescence emission filter (Semrock, FF01-425/527/685-25) for XcaMP-B and GcaMP6 recording. 575 nm shortpass filter (Edmund, 575 nm 25 mm diameter, O.D. 4.0 Shortpass filter) was directed into the tube lens to minimize direct LED emission detected by the camera. Imaging optical powers of 380 nm and 470 nm were used at the far end of the patch cord at 5 μW and 2.5 μW, respectively. The fluorescence image was focused onto the sensor of a sCMOS camera (Hamamatsu, ORCA-Flash4.0) through a tube lens (Thorlabs, AC254-035-A-ML).

A custom MATLAB (Mathworks, Natick, MA) GUI was written to control the sample illumination protocol as well as provide power modulation pulses to the LEDs (National Instruments, NI PCIe-6343-X) which temporally align the respective LED illumination with camera frame acquisition (HCImage, Hamamatsu). The generic illumination protocol would repeat a sequence of three-frame sampling periods: one isosbestic at 380 nm, one signal at 470 nm and one optogenetic at >470 nm (FIG. 7G). Maintaining a dedicated frame for optogenetic excitation faithfully removes any potential cross-excitation artifact from the isosbestic and signal sampling windows.

To quantify spectral cross-excitation of the opsin from signal illumination, the 470 nm LED was additionally pulsed during the optogenetic sampling period. The pulse duration of this additional illumination was matched to the signal pulse width (23 ms). The minimum excitation power for the sweep was equal to that used for the signal pulse (2.5 μW). The digital camera acquired data at a total of 30 Hz. Therefore, due to the sequential 3-frame sampling protocol, the isosbestic and signal samples were each acquired at 10 Hz and all optogenetic stimulation would similarly occur at a rate of 10 Hz. The duration of this 10 Hz optogenetic stimulation was 2 seconds. To quantify the excitation efficiency of the opsin to orange light at 594 nm, the associated LED was pulsed during the optogenetic sampling period (10 ms pulse width). For light-intensity sweeps, four samples at each power were randomly interleaved with a random ITI between 20 and 30 seconds. Optogenetic excitation in the NIR window at 720 nm and 750 nm were separately characterized using this same protocol (Inoue et al., 2019; Kim et al., 2016). For 720 nm optogenetic stimulation, the 594-nm LED was replaced with a 730-nm LED (M730L5, Thorlabs). The 730-nm laser was filtered with a 716-43 nm bandpass filter (Semrock, FF01-716/43-25). For 750 nm optogenetic stimulation, the 594-nm LED was replaced with a 750-nm laser (CivilLaser). The 750-nm laser was filtered with a 750-10 nm bandpass filter (Thorlabs, FB750-10).

The Pyr-PV impulse response data were acquired using the same optical configuration. A 594 nm LED was delivered using 10 ms pulse width and 1 mW of power. The pulse frequency (1, 2, 5, 10, 20 Hz) and pulse number (10, 20, 30, 40, 60, 80, 120) were controlled by TTL signals delivered by a microcontroller (Arduino, Uno) communicating with MATLAB (MathWorks). Four samples at each frequency and number were randomly interleaved with an ITI 30 seconds.

The fluorescence signal was calculated with custom written MATLAB scripts. We fit a double exponential to a thresholded version of the fluorescence time series and subtracted the best fit from the unthresholded signal to account for slow bleaching artifacts. Fluorescence signal was normalized within each mouse by calculating the ΔF/F as (F−baseline (F))/baseline (F), where the baseline was taken from the average during 5 s before optogenetic stimulation. Peak ΔF/F amplitude was calculated from the maximum value during 2 s after the stimulus cessation. Noise was calculated as the standard deviation of the ΔF/F fluctuation during 5 s before optogenetic stimulation. Signal-to-noise ratio (SNR) response was then computed as ΔF/F response divided by noise. Every measurement point (light intensity and wavelength) represents the average of four trials at 20-30 second intervals. The optical EPD50 in FIG. 7K was quantified by dividing the ΔF/F amplitude at each light intensity by the ΔF/F amplitude at 1 mW.

### Histology and Confocal Microscopy

To analyze the expression pattern of opsin and GcaMP, immunohistochemistry was performed in brain tissue removed from virus-injected mice. Animals were anesthetized and transcardially perfused with ice-cold 1×PBS followed by 4% paraformaldehyde (PFA) in PBS. Brains were dissected, post-fixed in the same fixatives overnight at 4° C. Tissues were cut into 60-μm-thick slices with a vibratome (Leica, VT1000) and floated in PBS. For immunohistochemistry, brain slices were blocked with 3% normal donkey serum/0.3% Triton X-100/PBS and incubated with primary antibody diluted in the blocking buffer at 4° C. overnight on a shaker. The antibody used was mouse monoclonal anti-HA tag (1:500, Fisher Scientific A26183). After washing with 0.3% Triton X-100/PBS, tissue sections were incubated with the secondary antibody, Alexa Fluor 647-conjugated donkey anti-mouse antibody (1:500, A-31571, Thermo Fisher Scientific) and DAPI for 2 h at R.T. and mounted on slides in a tissue-mounting medium containing anti-fade, Polyvinyl alcohol mounting medium with DABCO (Millipore Sigma). Confocal imaging of GcaMP fluorescence, HA antibody staining for localization of the opsin, and DAPI for cytoarchitecture was performed using a Leica TCS SP8 or TCS SP5 confocal scanning laser microscope with a 10×/NA-0.4 or 25×/NA-0.95 water objective. Co-localization was performed using 25× images by annotating GcaMP6m expressing cell body locations and then overlaying these annotations and verifying expression in the anti-HA image. Quantitative analysis of GcaMP expression level of individual mice was performed using 10× image (5-6 z slices at 3 μm intervals through each section) by annotating GcaMP6m expression. The fluorescence intensity of GcaMP6m was quantified from the slice with the highest fluorescence intensity by setting up a 400 μm square ROI directly under the fiber tract using ImageJ (NIH).

### Quantification and Statistical Analysis

For the electrophysiology experiments, pClamp 10.6 (Molecular Devices), Python, and Prism 7 (GraphPad) software were used to record and analyze data. Non-parametric tests (Wilcoxon rank-sum test and the signed rank test) were used for singular comparisons. For multiple comparisons, Kruskal-Wallis test was performed and was followed by Dunn's test for post-hoc comparisons. The peak photocurrent was identified as the largest difference in current in the interval from laser onset to laser offset. Tau-off was calculated by fitting a mono exponential curve to the waveform from laser offset to the baseline. Time-to-peak was calculated by measuring the time difference between laser onset and peak current.

To calculate action spectra, we first normalized photocurrents to the peak photocurrent for each cell. Then, these normalized spectra were averaged across cells to produce the action spectra for each opsin variant in both one-photon and two-photon measurements. To calculate EPD50, photocurrents were first normalized to the photocurrent elicited at the highest light power. Linear interpolation was then used to infer the light power level that produced 50% of the max photocurrent.

### Example 4—Crystal Structure Determination and Structure Based Mutagenesis

The unusual properties of ChRmine have opened up new avenues of investigation for optogenetics in the study of cell-specific activity within biological systems (Chen et al., 2021; Marshel et al., 2019); alongside extremely-large photocurrents, red-shifted actuation, and light sensitivity (Marshel et al., 2019), ChRmine exhibits virtually no Ca²⁺ conductance (FIG. 13A), a valuable property in long-timescale optogenetics applications for avoiding incidental induction of Ca²⁺ dependent plasticity. Here, we have revealed structure-function relationships underlying these remarkable properties, along with insights into evolution of microbial opsins. First, despite its fundamentally distinct channel-based mechanism, we find that ChRmine is surprisingly like the HsBR pump in terms of oligomerization number (FIG. 1C), overall monomeric structure (FIG. 1E), and proton acceptor (FIG. 2D), suggesting that ChRmine evolved from archaeal ion-pumping rhodopsins. In HsBR, D85 and D96 of DTD motif serve critical functions as relays for the pumping transfer of a single proton from the intracellular to extracellular side, in response to the absorbed photon. In ChRmine, these two Asps are conserved (D115 and D126) but not for H⁺-pumping relay purposes; rather, they form the two constriction sites (ICS and CCS) of the passive ion-conducting pore within the monomer. Thus, these two residues continue to play critical roles, but in gating the channel, rather than as relay points for H⁺ transport.

Arginine conformation in the dark state and the function of ion-transporting rhodopsins. R82 in HsBR is highly conserved among microbial rhodopsins, but two different conformations are seen in the dark state: outward-facing and parallel. In all structurally-resolved channelrhodopsins (C1C2 (PDB ID: 3UG9) (Kato et al., 2012), CrChR2 (PDB ID: 6EID) (Volkov et al., 2017), C1Chrimson (PDB ID: 5ZIH) (Oda et al., 2018), GtACR1 (PDB ID: 6CSM) (Kim et al., 2018), and ChRmine), the arginine residue faces outward. In contrast, in many ion-pumping rhodopsins, including HsBR (PDB ID: 5ZIM) (Hasegawa et al., 2018), HwBR (PDB ID: 4QID), cruxrhodopsin-3 (PDB ID: 4JR8) (Chan et al., 2014), deltarhodopsin (PDB ID: 4FBZ) (Zhang et al., 2013), GR (PDB ID: 6NWD) (Morizumi et al., 2019), Archaerhodopsin-1 (PDB ID: 1UAZ) (Enami et al., 2006), Archaerhodopsin-2 (PDB ID: 2EI4) (Yoshimura and Kouyama, 2008), PR from the Mediterranean Sea at a depth of 12 m (Med12BPR, PDB ID: 4JQ6) (Ran et al., 2013), PR from the Pacific Ocean near Hawaii at a depth of 75 m (HOT75BPR, PDB ID: 4KLY) (Ran et al., 2013), CsR (6GYH) (Fudim et al., 2019), HsHR (PDB ID: 1E12) (Kolbe et al., 2000), NpHR (PDB ID: 3A7K) (Kouyama et al., 2010), CiR (PDB ID: 5ZTK) (Yun et al., 2021), KR2 (PDB ID: 3X3C) (Kato et al., 2015), and schizorhodopsin 4 (PDB ID: 7E4G) (Higuchi et al., 2021), the tip of arginine runs parallel to the membrane and faces TM1. The arginine in the parallel conformation narrows or blocks the extracellular cavity of the ion-translocating pathway; thus, this conformation would contribute to preventing large ion flux in ion-pumping rhodopsins.

Notably, CsR (the outward proton-pumping rhodopsin from Coccomyxa subellipsoidea), also has arginine (R83) in the parallel conformation in the dark state (Fudim et al., 2019), and R83Q mutation or mutation of the adjacent tyrosine (Y57K) converts functionality from proton pump to proton channel (Vogt et al., 2015). Moreover, computational analysis of HsBR with

R82Q or Y57K mutation reveals that these mutations significantly change the conformation of R82Q or R82, respectively; most notably, R82 faces outward in the Y57K simulation (Vogt et al., 2015). These observations suggest that the conformation of the arginine in the dark state is one of the structural elements distinguishing channel- and pump-type rhodopsins. Interestingly, previous studies have reported that the arginine of some ion-pumping rhodopsins is maintained in the parallel conformation during the photocycle (Kouyama et al., 2015; Kovalev et al., 2020), but the corresponding arginine in HsBR transiently changes from parallel to outward-facing to facilitate proton release to the extracellular solvent (Kuhlbrandt, 2000; Nango et al., 2016). Since channelrhodopsins presumably evolved from ion-pumping rhodopsins (Inoue et al., 2015), these studies suggest that mutations accumulated near the arginine of ion-pumping rhodopsins gradually stabilized the outward-facing conformation; these rearrangements enlarged the extracellular cavity, enabling the large ion flux of channelrhodopsins.

The conformational change of the monomer pore during simulation: In addition to the opening of the trimer pore, we also observed that the size of the monomer pore increased during the light state simulation (FIG. 5E). Upon isomerization and proton transfer, both the retinal and D115 rotate away from the internal monomer pore, increasing the space within the monomer (FIG. 5F). While over the timescale of our simulation, the pore radius does not become large enough to allow for travel of ions through the monomers (as with the trimer pore), the monomeric changes follow the expected opening that would occur upon light activation.

Despite similarities to HsBR, ChRmine exhibits several atypical properties in its high-resolution structure, including its long twisted ECL1. ECL1 not only significantly distorts the architecture around the Schiff base but also enlarges the extracellular cavity within the monomer. Interestingly, the length and sequence of ECL1 are not highly conserved in this subfamily, and cation selectivity has been reported to be different between pump-like ChRs that we would predict to have long vs. short ECL1 domains (Sineshchekov et al., 2020). Furthermore, we report that ChRmine (with its long ECL1) is remarkably selective for monovalent cations (especially K⁺;

FIG. 13A), while HcKCR1 and HcKCR2 (recently discovered members of the same pump-like subfamily as ChRmine, with shorter ECL1 motifs) are reported to similarly exclude divalent cations and exhibit further selectivity for K⁺ over Na⁺ (Govorunova et al., 2021). Multiple molecular features within ECL1 may be relevant (for example, the conserved Arg residue (R112 in ChRmine) on ECL1 is replaced by Trp in HcKCR1 and HcKCR2), and ECL1 could be explored for roles in cation conduction and selectivity. Indeed, given the knowledge reported here regarding ChRmine's unusual structural and electrophysiological properties (especially the distinctive ECL1 feature (FIGS. 8A and 11B) and high monovalent cation selectivity shared by ChRmine's close relatives but not seen in other rhodopsins (Govorunova et al., 2021; Shigemura et al., 2019; FIG. 13A)), an updated inclusive name for this growing ChR family would be pump-like ChRs (PLCRs;

FIG. 8A-8B); these do not specifically resemble bacteriorhodopsin more than the other pumps, nor—as we now know—do they generally conduct all cations).

Proton donor and acceptor: In HsBR, D85 receives a proton from the protonated Schiff base and releases it to the extracellular bulk solvent. D96 receives a proton from the intracellular bulk solvent and provides it to the deprotonated Schiff base. These proton movements generate net flow of proton from the intracellular to extracellular side; these two functionally important residues, together with T89, are called DTD motif.

In GtCCR2, a ChRmine homolog in the BCCR family, both D85 and D96 are conserved (D87 and D98, respectively) but the proposed proton translocation pathway is completely different; GtCCR2 does not show outward proton-pumping activity (Sineshchekov et al., 2017), and the proton is shuttled back and forth between the Schiff base and D85. While the deprotonation and re-protonation of D98 are assumed to occur and the deprotonation would be necessary for the channel gating, D98 never gives the proton to the deprotonated Schiff base (Sineshchekov et al., 2017). If some channelrhodopsins retain residual pumping activity (Feldbauer et al., 2009), D85 homologs presumably could release a proton to the extracellular bulk solvent after receiving a proton from the Schiff base. However, in contrast to the proton acceptor, it remains elusive which residue works as a proton donor to the deprotonated Schiff base. D96 in HsBR is also conserved in ChRmine (D126), but is exposed to intracellular bulk solvent in our structure, and the calculated pKa of D126 is as low as 6.28. D126 may be unlikely to work as the sole proton donor (FIG. 3G) although is possible that the deprotonated Schiff base directly receives a proton from water molecules. Future studies will be needed to reveal the molecular nature of the proton donor, and understand how proton translocation is involved in channel gating of ChRmine.

Indeed, our current structural information of ChRmine has already provided a framework for further development of ChRmine-based optogenetic tools: hs, rs, and frChRmine (FIGS. 6A-6K and 7A-7R). It was a surprise to see the blue shoulder reduction, which had been extraordinarily difficult to address despite more than fifteen years of focused effort across many laboratories (Deisseroth and Hegemann, 2017). In the future, the structure of rs or frChRmine may therefore turn out to be of further value in elucidating the mechanism of this effect, and in porting these properties to other microbial opsins. Other design goals may include combining the key properties of rs, hs, and frChRmine with other ideas that have arisen during opsin engineering. For example, we previously found it productive to combine the mutations that enabled chloride flux (via conversion from cation-to-anion selectivity) with the mutations that gave rise to greatly increased light-sensitivity (via slowed kinetics), resulting in a single chloride-conducting step-function ChR exhibiting bistable inhibitory currents (Berndt et al., 2016). The high-resolution ChRmine structure and the new variants described here may point the way to such integration.

Published structures of ChRs were experimentally determined only using crystallography. However, we find that the combination of antibody and single-particle cryo-EM techniques (Wu et al., 2012) is powerful enough to determine the high-resolution structure of small proteins like ChRmine, thus representing a new and promising option for structural analysis of microbial rhodopsins alongside X-ray crystallography. The two technologies, as well as structure prediction methods, may complement each other and thus expedite structural biology of microbial rhodopsins, and the resulting information will lead to both further development of optogenetics and basic mechanistic understanding of these remarkable photoreceptor proteins.

A small population of early intermediates: initial structural changes in ChRmine: Our 2.0 Å cryo-EM map allowed accurate modeling of ATR and surrounding residues, but the C13 and C14 atoms of ATR and W223 showed weaker density in the region. Moreover, positive and negative Fo−Fc difference densities were observed around W223, suggesting that this cryo-EM density map contains information on a small population of early intermediate states (possibly the K intermediate; (FIGS. 9Q and 9R). While we could not detect further structural changes propagated from W223, the extent of the conformational change in ChRmine W223 was significantly larger than that in C1C2 and like that of HsBR (Oda et al., 2021; Weinert et al., 2019). Thus, it is concluded that the initial conformational changes of the ChRmine photocycle may be more like those of ion-pumping rhodopsins than of canonical chlorophyte channelrhodopsins.

Further structural work of value would include molecular dynamics simulations and structures of the intermediate states. Investigating cooperativity/allostery properties linking different monomers within the trimer could not only help tune light-response properties of optogenetic tools, but also could illuminate the basic science of ChR origins, including the conversion from trimer to dimer assembly-logic during the evolutionary separation of non-pump-like ChRs from the presumably pump-like ancestors. Recent discoveries and applications using optogenetics may be extended with these ChRmine variants to a broad range of opportunities, capitalizing upon the remarkably high photocurrents and light-sensitivity of the parent opsin, and the improved properties of the variants (Bansal et al., 2021; Sahel et al., 2021). Structural insight and structure-guided design of microbial opsins continue to open pathways for discovery and understanding, extending now to the third of the three major known ChR types: cation-conducting (Kato et al., 2012), anion-conducting (Kim et al., 2018; Kato et al., 2018), and pump-like channelrhodopsins.

Although the foregoing invention has been described in some detail by way of illustration and example for purposes of clarity of understanding, it is readily apparent to those of ordinary skill in the art considering the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

We claim:

1. A high-speed variant ChRmine protein having faster kinetic properties compared to a parent ChRmine protein, wherein the high-speed variant ChRmine protein has one or more amino acid substitutions compared to the parent ChRmine.

2. The high-speed variant ChRmine protein according to claim 1, comprising one or more amino acid substitutions in the Schiff base counterion of the parent ChRmine protein.

3. The high-speed variant ChRmine protein according to claim 1, comprising one or more amino acid substitutions that alter the pore electrostatic potential of the parent ChRmine protein.

4. The high-speed variant ChRmine protein according to claim 3, wherein the one or more amino acid substitutions that alter the pore electrostatic potential of the parent ChRmine protein are selected from: 33^{rd} histidine or a corresponding position; 92^{nd} aspartate or a corresponding position; 154^{th} glutamate or a corresponding position; 158^{th} glutamate or a corresponding position, 242^{nd} aspartate or a corresponding position, and 246^{th} glutamate or a corresponding position.

5. The high-speed variant ChRmine protein according to claim 4, wherein: the 33^{rd} histidine or a corresponding position is substituted with histidine, when the corresponding amino acid is not histidine, arginine, or lysine.

6. The high-speed variant of ChRmine protein according to claim 4, wherein: each of the 92^{nd} aspartate or a corresponding position, 154^{th} glutamate or a corresponding position, 158^{th} glutamate or a corresponding position, 242^{nd} aspartate or a corresponding position, and 246^{th} glutamate or a corresponding position is substituted independently of each other with aspartate, glutamate, asparagine, or glutamine.

7. The high-speed variant ChRmine protein according to any one of claims 1 to 6, wherein the parent ChRmine protein has a sequence selected from SEQ ID NOs: 1 to 29 or a sequence having at least 80% sequence identity to a sequence selected from SEQ ID NOs: 1 to 29.

8. The high-speed variant ChRmine protein according to any one of claims 1 to 7, wherein, compared to the parent ChRmine protein, the high-speed variant ChRmine protein has a substitution at the histidine residue in the 33^{rd} position or the corresponding residue in the first transmembrane domain of the parent ChRmine protein.

9. The high-speed variant ChRmine protein according to claim 8, wherein the high-speed variant ChRmine protein has an arginine substitution at the histidine residue in the 33^{rd} position or the corresponding residue in the first transmembrane domain of the parent ChRmine protein.

10. The high-speed variant ChRmine protein according to any one of claims 1 to 9, having the sequence of SEQ ID NO: 30 or a sequence having at least 80% sequence identity to SEQ ID NO: 30, wherein the variations in the sequence having at least 80% sequence identity to SEQ ID NO: 30 exclude the amino acid substitution used to produce the high-speed variant ChRmine protein.

11. The high-speed variant ChRmine protein according to any one of claims 1 to 10, having the sequence of SEQ ID NO: 30.

12. A red-shifted variant ChRmine protein having a red-shifted spectrum compared to a parent ChRmine protein, wherein the red-shifted variant ChRmine protein has one or more amino acid substitutions compared to the parent ChRmine protein.

13. The red-shifted variant ChRmine protein according to claim 12, comprising one or more amino acid substitutions in the retinal binding pocket (RBP) of the parent ChRmine protein.

14. The red-shifted variant ChRmine protein according to claim 13, wherein the one or more amino acid substitutions in the RBP of the parent ChRmine protein comprise substitutions in one or more of: 146^{th} isoleucine or a corresponding position; 174^{th} glycine or a corresponding position; 178^{th} phenylalanine or a corresponding position.

15. The red-shifted variant ChRmine protein according to claim 13, wherein: the substitution at the 146^{th} isoleucine or a corresponding position is with a serine, cysteine, threonine, or methionine; the substitution at the 174^{th} glycine or a corresponding position is with a serine, cysteine, threonine, or methionine; or the substitution at the 178^{th} phenylalanine or a corresponding position is with phenylalanine, when the corresponding amino acid is not phenylalanine, tyrosine, or Tryptophan.

16. The red-shifted variant ChRmine protein according to any one of claims 12 to 15, wherein the parent ChRmine protein has a sequence selected from SEQ ID NOs: 1 to 29 or a sequence having at least 80% sequence identity to a sequence selected from SEQ ID NOs: 1 to 29.

17. The red-shifted variant ChRmine protein according to any one of claims 12 to 16, wherein, compared to the parent ChRmine protein, the red-shifted variant ChRmine protein has one or both of: i) a substitution at the isoleucine residue in the 146^{th} position or the corresponding residue in the fourth transmembrane domain of the parent ChRmine protein; and ii) a substitution at the glycine residue in the 174^{th} position or the corresponding residue in the fifth transmembrane domain of the parent ChRmine protein.

18. The red-shifted variant ChRmine protein according to claim 17, wherein, compared to the parent ChRmine protein, the red-shifted variant ChRmine protein has one or both of: i) a methionine substitution at the isoleucine residue in the 146^{th} position or the corresponding residue in the fourth transmembrane domain of the parent ChRmine protein; and ii) a serine substitution at the glycine residue in the 174^{th} position or the corresponding residue in the fifth transmembrane domain of the parent ChRmine protein.

19. The red-shifted variant ChRmine protein according to any one of claims 12 to 18, having the sequence of SEQ ID NO: 31 or a sequence having at least 80% sequence identity to SEQ ID NO: 31, wherein the variations in the sequence having at least 80% sequence identity to SEQ ID NO: 31 exclude the amino acid substitution used to produce the red-shifted variant ChRmine protein.

20. The red-shifted variant ChRmine protein according to any one of claims 12 to 18, having the sequence of SEQ ID NO: 31.

21. A high-speed and red-shifted variant ChRmine protein having faster kinetics and red-shifted spectrum compared to a parent ChRmine protein, wherein the high-speed and red-shifted variant ChRmine protein has one or more amino acid substitutions compared to the parent ChRmine protein.

22. The high-speed and red-shifted variant ChRmine protein according to claim 21, comprising: i) one or more amino acid substitutions in Schiff base counterion of the parent ChRmine protein or one or more amino acid substitutions that alter the pore electrostatic potential of the parent ChRmine protein, and ii) one or more amino acid substitutions in the retinal binding pocket (RBP) of the parent ChRmine protein.

23. The high-speed and red-shifted variant ChRmine protein according to claim 21 or 22, comprising: i) one or more amino acid substitutions that alter the pore electrostatic potential of the parent ChRmine protein and ii) one or more amino acid substitutions in the retinal binding pocket (RBP) of the parent ChRmine protein.

24. The high-speed and red-shifted variant ChRmine protein according to claim 23, wherein the one or more amino acid substitutions that alter the pore electrostatic potential are selected from: 33^{rd} histidine or a corresponding position; 92^{nd} aspartate or a corresponding position; 154^{th} glutamate or a corresponding position; 158^{th} glutamate or a corresponding position, 242^{th} aspartate or a corresponding position, and 246^{th} glutamate or a corresponding position.

25. The high-speed and red-shifted variant ChRmine protein according to claim 24, wherein: 33^{rd} histidine or a corresponding position is substituted with histidine, when the corresponding amino acid is not histidine, arginine, or lysine.

26. The high-speed and red-shifted variant ChRmine protein according to claim 24, wherein: each of 92^{nd} aspartate or a corresponding position, 154^{th} glutamate or a corresponding position, 158^{th} glutamate or a corresponding position, 242^{nd} aspartate or a corresponding position, and 246^{th} glutamate or a corresponding position is independently substituted with aspartate, glutamate, asparagine, or glutamine.

27. The high-speed and red-shifted variant ChRmine protein according to any one of claims 22 to 26, wherein the one or more amino acid substitutions in the RBP of the parent ChRmine protein comprise substitutions in one or more of: 146^{th} isoleucine or a corresponding position; 174^{th} glycine or a corresponding position; and 178^{th} phenylalanine or a corresponding position.

28. The high-speed and red-shifted variant ChRmine protein according to claim 27, wherein: the substitution at the 146^{th} isoleucine or a corresponding position is with a serine, cysteine, threonine, or methionine; the substitution at the 174^{th} glycine or a corresponding position is with a serine, cysteine, threonine, or methionine; or the substitution at the 178^{th} phenylalanine or a corresponding position is with phenylalanine, when the corresponding amino acid is not phenylalanine, tyrosine, or Tryptophan.

29. The high-speed and red-shifted variant ChRmine protein according to any one of claims 21 to 28, wherein the parent ChRmine protein has a sequence selected from SEQ ID NOs: 1 to 29 or a sequence having at least 80% sequence identity to a sequence selected from SEQ ID NOs: 1 to 29.

30. The high-speed and red-shifted variant ChRmine protein according to any one of claims 21 to 29, wherein, compared to the parent ChRmine protein, the high-speed and red-shifted variant ChRmine protein has one or more of: i) a substitution at the histidine residue in the 33^{rd} position or the corresponding residue in the first transmembrane domain of the parent ChRmine protein; ii) a substitution at the isoleucine residue in the 146^{th} position or the corresponding residue in the fourth transmembrane domain of the parent ChRmine protein; and iii) a substitution at the glycine residue in the 174^{th} position or the corresponding residue in the fifth transmembrane domain of the parent ChRmine protein.

31. The high-speed and red-shifted variant ChRmine protein according to claim 30, wherein, compared to the parent ChRmine protein, the high-speed and red-shifted variant ChRmine protein has one or more of: i) an arginine substitution at the histidine residue in the 33^{rd} position or the corresponding residue in the first transmembrane domain of the parent ChRmine protein; ii) a methionine substitution at the isoleucine residue in the 146^{th} position or the corresponding residue in the fourth transmembrane domain of the parent ChRmine protein; and iii) a serine substitution at the glycine residue in the 174^{th} position or the corresponding residue in the fifth transmembrane domain of the parent ChRmine protein.

32. The high-speed and red-shifted variant ChRmine protein according to any one of claims 21 to 31, having the sequence if SEQ ID NO: 32 or a sequence having at least 80% sequence identity to SEQ ID NO: 32, wherein the variations in the sequence having at least 80% sequence identity to SEQ ID NO: 32 exclude the amino acid substitution used to produce the high-speed and red-shifted variant ChRmine protein.

33. The high-speed and red-shifted variant ChRmine protein according to any one of claims 21 to 31, having the sequence of SEQ ID NO: 32.

34. A nucleic acid encoding for a variant ChRmine protein according to any one of the preceding claims.

35. A genetically modified cell comprising the nucleic acid according to claim 34.

36. An optogenetic method comprising:
genetically modifying a subject to express in the subject's brain cells the variant ChRmine protein according to any one of claims 1 to 33,
applying stimulating light to the subject's brain, and
imaging the subject's brain.

37. The optogenetic method according to claim 36, wherein the subject is a mammal.

38. The optogenetic method according to claim 37, wherein the mammal is a rodent, a primate, a bovine, a porcine, a feline, or a canine.

39. A method comprising: genetically modifying a subject to express in a cell and/or organ the variant ChRmine protein according to any one of claims 1 to 33.

40. The method of claim 39, further comprising applying stimulating light to the modified cell and/or organ, and imaging the subject's cell and/or organ.

41. The method of claim 40, wherein the cell and/or organ can belong to the cardiovascular system, the gastrointestinal system, urinary system, the respiratory system, the reproductive system, the musculoskeletal system, or the pancreatic/endocrine system.


SEQUENCE LISTING (32 sequences)

SEQ ID NO: 1 - protein, 309 aa, Tiarina fusus
MAHAPGTDQMFYVGTMDGWYLDTKLNSVAIGAHWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAILMSGVLAEFYAEGDPRLRNGAYAWYGFGCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLRRLS

SEQ ID NO: 2 - protein, 265 aa, Hyphochytrium catenoides
MPFYDSRPPEGWPRGSVNDMDYPLLGSICAISAIAIAGSGIWMLYRLDLGMGYSCKPYKSGRAPEVNSISGIVCLLCGTMYAAKSFDFFDGGGTPFSLNWYWYLDYVFTCPLLIVDFAFTLDIPQKLRYSIAVFVALWCAVAAFATPSAFRFAYYALGCCWFIPLSLFLIRDVKKRYQVYPPKCQRLLFWACVVFFGFWPLFPLLFIFSWQGSGHISRQAYYIIHAFLDLVCKSIFGFLMTFFRLELEEHTEVQGLPLKEPKVMD

SEQ ID NO: 3 - protein, 265 aa, Hyphochytrium catenoides
MPFYDSRPPEGWPKGSINDMDYPLLGSICAVCCVFVAGSGIWMLYRLDLGMGYSCKPYKSGRAPEVNSLSGIICLLCGTMYAAKSFDFFDGGGTPFSLNWYWYLDYVFTCPLLILDFAFTLDLPHKIRYFFAVFLTLWCGVAAFVTPSAYRFAYYALGCCWFTPFALSLMRHVKERYLVYPPKCQRWLFWACVIFFGFWPMFPILFIFSWLGTGHISQQAFYIIHAFLDLTCKSIFGILMTVFRLELEEHTEVQGLPLNEPETLS

SEQ ID NO: 4 - protein, 331 aa, Rhodomonas abbreviata
MVSEGYGDKYLEGSMRGWYTPCTFGTPAIMAHWVTFLFLFVCTMYLARDSWTARGPGGRERWYAGYHEDYNISLYVNLFAMIAYFGKIVADSVGHNYNNVGPLVIGLGNYRYADYMLTCPLLAYDLMSQVRAPFKITGGVLIFCVLLTGTATNYYPGDEMRAGSMAWFIFGCFLYFVAYYLFYTVVKIQYTRLLALAASTEAKKAFMPLKLALYTFFSIWIVFPIVWLLGHQGLNILSNDAQECLHCACDLTAKSFYGFALAKYRKYFDKKMFDMLTELGIDGEEGLEHLEQDLKNQDIGDGKADIKRMSTTRGPGLSVISVNKHHPAFLA

SEQ ID NO: 5 - protein, 334 aa, Rhodomonas salina
MVLDLPEDFARMHLEGTMEGWYQPTTFGSAAIVAHWITFLFLTLCTMYLARDSWTAKGPGGKELYFAGYHEEYNISLYVNLFAMIAYFGKVVADSMGHNYNNVGPMIIGLGNYRYADYMLTCPLLAWDLMAQVRAPYKITGAILIFCVLLSGAATNFYPGEQMKQGAIAWFCFGCFLYAAAYYLFFSIVKKQYSRLVALSANTEAKKAFMPLKLALYTFFSIWVVFPIVWILGYHGLNILTNDAQECLHCACDLIAKSFYGFALAKYRTYFDKKMYDMLEELGVDAEEGLEHLEKDLKEVDDPNNVNNLKRLSVAASKTRQHNHANINAFSAEP

SEQ ID NO: 6 - protein, 316 aa, Rhodomonas abbreviata
MPMFGGRRSESEKAPPIEGDERNILIADIVGFSCLGITAFILNYRLKAFKGPRDDDKYFVGYREEKMLALLINLFATVGYWAKICARYDSNMGSSVDITTYEYLDYLATCPLVTLDMLWTLNLPYKFTSGAMIFLCILCAFASEITAGTARWMWFGFGFILFLYVWTTVGSLVKMRLQQLKTLTDQGVLVPGQTSLKIATATFFCLWLGYPLLWLILDLGGMTEFQAEICHVGFDVVVKSLYGLGMLEFVMQADRIHFEFETLSPPDDYGSESSKNYGKDLESGQQPSSFDTFKGTHAAKEEKEEKEDEEDAHKTD

SEQ ID NO: 7 - protein, 309 aa, Rhodomonas salina
MAAGNEHFETPQEARMVLIADYVAFCCLAACAFFLHRRIVMFRGPTGDDHYFMGYREEKMLCVLITIFAAIGYWSRITARVQGSHKALDLSTLQYVDYLATCPLVTVDMLWSLNLPYKFTWGALVFLCILSAFACDAMEGQARWMWFGVGMGLFSYVWISVGRLVKFRLTQLAALRAEGVLKVDIEYPLKVGTATFFFLWFAYPLMWLLEEFDVINHAKSEVAHVVFDVVVKSVYAICLQEFGFRCDRTYFEFPSMSVTVVPDEEYQTKNEHAMDEYANERLQDAWHKLPVNETQRHTVPKSWHCQGSM

SEQ ID NO: 8 - protein, 300 aa, Guillardia theta
MVESSAVIAANWISFLVIAGSFVVLCFISLRYKGPGGNENYYNGFREQNMLTVIINLWCALAYFAKVLQSHSDDDGFVPLTKIPYLDYATTCPLLTLDLMWCLDAPYKITSAVLVFTVMITGVACSLAVAPYSFYWFAMGMVLFIFTYVLMLSIVRERLEFITQCAHDSNAKRSIKHLKAAVIIYFGIWPIFAILWLLSYRAANVISNDTNHILHCILDVIAKSCFGFVLLHFKMYFDKKLLESGVHEDEFAEYSKEVSKHREEKPWRKTASSYPESDYDEPVYMNGEDLNLNDPPAWGG

SEQ ID NO: 9 - protein, 300 aa, Guillardia theta
MVASSAVITANWISFLAISASFIILLVISLRYKGPGGTESFYNGFKEQNMLTVFINLWCALAYFAKVLQSHSNDNGFAPLTVIPYVDYCTTCPLLTLDLLWCLDAPYKISSAVLVFTCLVIAVACSLAVAPFSYCWFAMGMVLFTFTYVFILSIVRQRLDFFTLCARDSNAKQSLKHLKTAVFIYFGIWLLFPLLWLLSYRAANVISNDINHIFHCILDVIAKSVYGFALLYFKMYFDKKLIESGIDEDDFAKFSKVVTTHRSEEKQKRKPAVSQSPKMYYDEAAYQDGEVESNLQSKIR

SEQ ID NO: 10 - protein, 300 aa, Guillardia theta
MVSALDQNGPQYLQNPIVIAADWIGFIALFGSSLAVAYKLVTFKGPDQDDVYFFGYREEKMISVFVNLFAALAYWAKLASHANGDVGPAASVTTYKYLDYLFTCPLLTIDLLWCLNLPYKFTFGAIVAVCILCAFMASVIPPPARYMWFGMGITVFSAAWFNILKLVRMRLEQFVSKEAKKVRQSLKVACMTYFFIWLGYPTLWVLGDAGVLDSVVSALLHTFLDVFSKSIYGFALLHFVMRTDKREFIFVPLKPSVEKIVVEDDESTSSRAESTRASKSKIMIGSRVKKLRSSATDELR

SEQ ID NO: 11 - protein, 367 aa, Guillardia theta
MTTSAPSLSDPNWQYGMGGWNNPRLPNFNLHDPTVIGVDWLGFLCLLGASLALMYKLMSFKGPDGDQEFFVGYREEKCLSIYVNLIAAITYWGRICAHFNNDMGLSLSVNYFKYLDYIFTCPILTLDLLWSLNLPYKITYSLFVGLTIACGVFCNAFEPPARYLWFMFGCFIFAFTWISIIRLVYARFQQFLNEDAKKIRAPLKLSLTLYFSIWCGYPALWLLTEFGAISQLAAHVTTVIMDVAAKSVYGFALLKFQLGVDKRDVWLDELKSVRYRDVVPQIRPSKTREGRMEYSEDGDFMRPSKGKRAEGDYMNPRWDHHDDGRRLPDSREMDEQVHEKDQEISSTMKQIADLNKQLSAMQESEAV

SEQ ID NO: 12 - protein, 228 aa, Halobacterium salinarum
TGRPEWIWLALGTALMGLGTLYFLVKGMGVSDPDAKKFYAITTLVPAIAFTMYLSMLLGYGLTMVPFGGEQNPIYWARYADWLFTTPLLLLDLALLVDADQGTILALVGADGIMIGTGLVGALTKVYSYRFVWWAISTAAMLYILYVLFFGFTSKAESMRPEVASTFKVLRNVTVVLWSAYPVVWLIGSEGAGIVPLNIETLLFMVLDVSAKVGFGLILLRSRAIFGE

SEQ ID NO: 13 - protein, 333 aa, Chlamydomonas reinhardtii
STGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVSSEDLYFQ

SEQ ID NO: 14 - protein, 315 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 15 - protein, 347 aa, Chlamydomonas reinhardtii
MSRRPWLLALALAVALAAGSAGASTGSDATVPVATQDGPDYVFHRAHERMLFQTSYTLENNGSVICIPNNGQCFCLAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 16 - protein, 277 aa, Guillardia theta
ITCDPAIYGEWSRENQFCVEKSLITLDGIKYVQLVMAVVSACQVFFMVTRAPKVPWEAIYLPTTEMITYSLAFTGNGYIRVANGKYLPWARMASWLCTCPIMLGLVSNMALVKYKSIPLNPMMIAASSICTVFGITASVVLDPLHVWLYCFISSIFFIFEMVVAFAIFAITIHDFQTIGSPMSLKVVERLKLMRIVFYVSWMAYPILWSFSSTGACIMSENTSSVLYLLGDALCKNTYGILLWATTWGLLNGKWDRDYVKGRNVDGTLMPEYEQDLE

SEQ ID NO: 17 - protein, 712 aa, Chlamydomonas reinhardtii
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGICRDLVRYLAWLYFCSWAMFPVLFLLGPEGFGHINQFNSAIAHAILDLASKNAWSMMGHFLRVKIHEHILLYGDIRKKQKVNVAGQEMEVETMVHEEDDETQKVPTAKYANRDSFIIMRDRLKEKGFETRASLDGDPNGDAEANAAAGGKPGMEMGKMTGMGMGMGAGMGMATIDSGRVILAVPDISMVDFFREQFARLPVPYELVPALGAENTLQLVQQAQSLGGCDFVLMHPEFLRDRSPTGLLPRLKMGGQRAAAFGWAAIGPMRDLIEGSGVDGWLEGPSFGAGINQQALVALINRMQQAKKMGMMGGMGMGMGGGMGMGMGMGMGMAPSMNAGMTGGMGGASMGGAVMGMGMGMQPMQQAMPAMSPMMTQQPSMMSQPSAMSAGGAMQAMGGVMPSPAPGGRVGTNPLFGSAPSPLSSQPGISPGMATPPAATAAPAAGGSEAEMLQQLMSEINRLKNELGE

SEQ ID NO: 18 - protein, 837 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEEDDTVKQSTAKYASRDSFITMRNRMREKGLEVRASLDAGGGDSGMEAGGGGAAHAQPHMAKPGTELGKTMSASFTNGAATSLEPGRVILAVPDISMVDFFREQFAQLPVPYEVVPALGAENTVQLVQQAAMLGGCDFVLMHPEFLRDRGPTGLLPQVKMMGQRTAAFGWSQMGPMRDLIESSGVGAWLEGPSFGSGISQAALQQLVVKMQQAKRMAAMGSMMGGGMGNGMGMGMGMGMGMGMGNGMGNGMGMGNGMGNGMGMGNGMGNGMGMGNGMGMGNGMGMGNGMGMGNGMGNGMGNGMGMGNGMGNGMGNGMGNGMGNGMGNGMGMGNGMGMGNGMGNGMGNGMGNGMGNGMGMMTPGAMGMGMGGMGNLAAAAGNAMYGGGGGGGGSTMGSGNAAMMTGLVMGGGNGVGAGPGGVVANLGSSALQPQSQMMGGGNVVGMSSPQLQLQQSSSMPLGGLAPNRIGNNPLFGAAPSPLHSQPGASPTGLSSPQLGMGAMLPAGTSVGAGGGSVGPTETDMLQQLMTEINRLKDELGE

SEQ ID NO: 19 - protein, 747 aa, Volvox carteri
MDHPVARSLIGSSYTNLNNGSIVIPSDACFCMKWLKSKGSPVALKMANALQWAAFALSVIILIYYAYATWRTTCGWEEVYVCCVELTKVVIEFFHEFDEPGMLYLANGNRVLWLRYGEWLLTCPVILIHLSNLTGLKDDYNKRTMRLLVSDVGTIVWGATAAMSTGYIKVIFFLLGCMYGANTFFHAAKVYIESYHTVPKGLCRQLVRAMAWLFFVSWGMFPVLFLLGPEGFGHLSVYGSTIGHTIIDLLSKNCWGLLGHFLRLKIHEHILLYGDIRKVQKIRVAGEELEVETLMTEEAPDTVKKSTAQYANRESFLTMRDKLKEKGFEVRASLDNSGIDAVINHNNNYNNALANAAAAVGKPGMELSKLDHVAANAAGMGGIADHVATTSGAISPGRVILAVPDISMVDYFREQFAQLPVQYEVVPALGADNAVQLVVQAAGLGGCDFVLLHPEFLRDKSSTSLPARLRSIGQRVAAFGWSPVGPVRDLIESAGLDGWLEGPSFGLGISLPNLASLVLRMQHARKMAAMLGGMGGMLGSNLMSGSGGVGLMGAGSPGGGGGAMGVGMTGMGMVGTNAMGRGAVGNSVANASMGGGSAGMGMGMMGMVGAGVGGQQQMGANGMGPTSFQLGSNPLYNTAPSPLSSQPGGDASAAAAAAAAAAATGAASNSMNAMQAGGSVRNSGILAGGLGSMMGPPGAPAAPTAAATAAPAVTMGAPGGGGAAASEAEMLQQLMAEINRLKSELGE

SEQ ID NO: 20 - protein, 325 aa, Stigeoclonium helveticum
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 21 - protein, 291 aa, Guillardia theta
MASQVVYGEWASTHTECYNMSRIDSTFVSLLQLVWAVVSGCQTIFMISRAPKVPWESVYLPFVESITYALASTGNGTLQMRDGRFFPWSRMASWLCTCPIMLGQISNMALVKYKSIPLNPIAQAASIIRVVMGITATISPAEYMKWLFFFFGATCLVFEYSVVFTIFQVGLYGFESVGTPLAQKVVVRIKMLRLIFFIAWTMFPIVWLISPTGVCVIHENVSAILYLLADGLCKNTYGVILWSTAWGVLEGKWDPACLPGQEKPEADDPFGLNHEKNAPPNDEVNIRMFGR

SEQ ID NO: 22 - protein, 297 aa, Rhodomonas lens
MALYTSQFGIAYGSWEAENDDCLSMGIASGAPVKIIQLCWAVVSLCQVIFMLTRAPRVAWEAIYLPLCETITYFLAFSGNGYLRMADGQIFPWARMASWLVTCPVMLGQVSAMALIKYKSLPLNNMMVAASLIRVVMGISATITPNEQLKWMFFFFGVSMLTFEYFCVFTIFSITIADFQEIDTPRSRFVVGRLKILRIVFHSAWTAFPILWVVSSTGLCVLSENISSVCYMLADAACKNGYGLVLWNTTWGHLNGKWDREFAKNADKDGEMKAIEEGAAGPAADAKKDEDIKLFGK

SEQ ID NO: 23 - protein, 262 aa, synthetic construct
MVPIYATFQLFAVLITAATLVFYVSQLRNGNKGDWEPIYVSGVEFIAYLMALVLPDGYAFRSFQDDSGIITVPVFRYVSWLATCPIILKVLVTVISHEDNIDQDVILRLMLLITWIELLGFMGSMYVGMLKVFCIALAIILCIYMYYIICTTWNKNHHESLLLSYPKERFELMALLMVSWIVFPILYIVGPECFGLISNQFSIIGHVIGDVISKNLWGLMAWKLRLKIKDGQPMEHVNPLKIVSNDGTNTMENYPLDKIIQT

SEQ ID NO: 24 - protein, 536 aa, Pyramimonas melkonianii
MMMMAPPESTDAEIAIRKGIEWIDFIMSVSLFAFFYYQKTVKKRCKFEVLYVTGVGGISDFIKVTAATVVPARLTIQENGIKFGWSRYFGWLTTCPVLLIHLSNLAGKEVFDVRRMMKILVAYQLLMCSGATAGMTDKDNIKWGFYLFAVFWLFVVFRYGKSIFAEAKMAMPKRAAPVLYRIMGIFYGGWAAFGITWFMGPEGVGWISPDAAKAVYAVLDILTKNVYAMHGWYLRWYILRKHNKPEEFVEQENVAEENQPKELKILLVEQDEVFGYFFYNMLVQHGSKVDLAKNLSELMAKTSNVGIQYDMIMINHDLAKQNNYQIMFEIRKSLFMLPVISYGRNIPKDEMDNRTATGIDDFLVAPFPDDEVRNKMTKWSRRASVDPSLIMNMSNMAAMANSATAIPGKGAAPTMSFGTGVRHSDAGPGVNKGGFKMGAIGEEATDCDNWVEPRQGARHPPANDGSADLNSKLDLLMRQLEDVKSSTSAQTREMQQKMDQQQKAFEQQQQQMMMSQQMMQQPQQMMQQPQQMMQQP

SEQ ID NO: 25 - protein, 409 aa, unidentified
MSFDQTICKHIQFIDLLLSIILFIFFYYQKIVKRRCKFEVLYVTGVGSLTDLVKLTITNELPASYVIENNGVLFNWSQFLGWMITCPVLLIHLSNLAGKDVFDVRRMMKILVIYQILMVSGATASMCNNDLKWLFFGVSLISLLKIYIYAHNIFQESREIMPKRANVVINWISFVFYVSWSGFGVSWFFSPNGISFFSDYTTKALFAFFDIMSKNVYSMLGWYLRWYIIRKFDNPEEFVEQYQEEDEKKINILLVENNAIYSHYFDNILIQHGCTVDVSKNIDELLFMTSNNDRQYDMIFMNKTMAIENSYKMMYEIRKRMFMLPVIVYGRDITEYDMMNRNNTGIDDFLIAPFPDDLLKKKLLQWSRRASINPLLTTNLNTNSNNIMNHIDIVIKSLNDLRSSKQFVS

SEQ ID NO: 26 - protein, 253 aa, Halobacterium salinarum
AVRENALLSSSLWVNVALAGIAILVFVYMGRTIRPGRPRLIWGATLMIPLVSISSYLGLLSGLTVGMIEMPAGHALAGEMVRSQWGRYLTWALSTPMILLALGLLADVDLGSLFTVIAADIGMCVTGLAAAMTTSALLFRWAFYAISCAFFVVVLSALVTDWAASASSAGTAEIFDTLRVLTVVLWLGYPIVWAVGVEGLALVQSVGATSWAYSVLDVFAKYVFAFILLRWVANNERTVAVAGQTLGTMSSDD

SEQ ID NO: 27 - protein, 235 aa, unidentified
MGEILAVDDYVGISFWLAAAIMLASTVFFFVERSDVPVKWKTSLTVAGLVTGVAFWHYLYMRGVWIYAGETPTVFRYIDWLITVPLQIIEFYLIIAAVTAISSAVFWKLLIASLVMLIGGFIGEAGLGDVVVWWIVGMIAWLYIIYEIFLGETAKANAGSGNAASQQAFNTIKWIVTVGWAIYPIGYAWGYFGDGLNEDALNIVYNLADLINKAAFGLAIWAAAMKDKETSTSHA

SEQ ID NO: 28 - protein, 273 aa, Salinibacter ruber
MLQELPTLTPGQYSLVFNMFSFTVATMTASFVFFVLARNNVAPKYRISMMVSALVVFIAGYHYFRITSSWEAAYALQNGMYQPTGELFNDAYRYVDWLLTVPLLTVELVLVMGLPKNERGPLAAKLGFLAALMIVLGYPGEVSENAALFGTRGLWGFLSTIPFVWILYILFTQLGDTIQRQSSRVSTLLGNARLLLLATWGFYPIAYMIPMAFPEAFPSNTPGTIVALQVGYTIADVLAKAGYGVLIYNIAKAKSEEEGFNVSEMVEPATASA

SEQ ID NO: 29 - protein, 290 aa, Dokdonia eikasta
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNKELENLYFQSGSHHHHHH

SEQ ID NO: 30 - protein, 309 aa, synthetic construct
MAHAPGTDQMFYVGTMDGWYLDTKLNSVAIGARWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAILMSGVLAEFYAEGDPRLRNGAYAWYGFGCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLRRLS

SEQ ID NO: 31 - protein, 309 aa, synthetic construct
MAHAPGTDQMFYVGTMDGWYLDTKLNSVAIGAHWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAMLMSGVLAEFYAEGDPRLRNGAYAWYGFSCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLRRLS

SEQ ID NO: 32 - protein, 309 aa, synthetic construct
MAHAPGTDQMFYVGTMDGWYLDTKLNSVAIGARWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAMLMSGVLAEFYAEGDPRLRNGAYAWYGFSCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLRRLS